(19) United States
(12) Patent Application Publication
(10) Pub. No.: US 2022/0348619 A1
(43) Pub. Date: Nov. 3, 2022

(54) RARE EARTH ELEMENT (REE)-BINDING PROTEINS

(71) Applicant: Ginkgo Bioworks, Inc., Boston, MA (US)

(72) Inventors: Patrick Boyle, Boston, MA (US); Ramya L. Prathuri, Boston, MA (US); Nathan W. Schmidt, Boston, MA (US); Emily E. Wrenbeck, Boston, MA (US); Mark David Simon, Boston, MA (US)

(73) Assignee: Ginkgo Bioworks, Inc., Boston, MA (US)

(21) Appl. No.: 17/620,654

(22) PCT Filed: Jun. 19, 2020
(86) PCT No.: PCT/US2020/038808

§ 371 Date: Dec. 17, 2021

Related U.S. Application Data
(60) Provisional application No. 62/865,090, filed on Jun. 21, 2019.

(51) Int. Cl. C07K 14/47; C07K 7/08; C22B 3/18; C22B 59/00

(52) U.S. Cl. CPC C07K 14/4728; C07K 7/08; C22B 3/18; C22B 59/00

(57) ABSTRACT

Described in this disclosure are rare earth element (REE)-binding proteins (e.g., lanthanide-binding proteins), host cells expressing the REE-binding proteins, and methods of recovering REEs.

### CROSS REFERENCE TO RELATED APPLICATIONS

This application claims the benefit under 35 U.S.C. § 119(e) of U.S. Provisional Application No. 62/865,090, filed Jun. 21, 2019, entitled “Rare Earth Element (REE)-Binding Proteins,” the entire disclosure of which is hereby incorporated by reference.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Jun. 19, 2020, is named G091970032WO00-SEQ-OMJ.txt and is 1.40 megabytes in size.

### FIELD OF THE INVENTION

The disclosure relates to methods of recovering rare earth elements, including lanthanides.

### BACKGROUND

Rare earth elements (REEs) are a group of seventeen metals that include lanthanides, yttrium (Y), and scandium (Sc). The lanthanide series of chemical elements comprises elements with atomic numbers 57 through 71 (e.g., lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu)). As a key component of many manufactured goods, including electronic devices, REEs are highly sought-after raw materials. Although many REEs are relatively abundant in the earth's crust with the exception of radioactive promethium, REEs are usually dispersed and are not concentrated in rare-earth minerals. Therefore, economical extraction of REEs has been difficult.

### SUMMARY

The present disclosure is based, at least in part, on the identification of new rare earth element (REE)-binding proteins.

Aspects of the disclosure relate to methods of recovering a rare earth element (REE) from a sample. In some embodiments, recovering an REE from a sample comprises introducing an REE-binding protein to the sample. In some embodiments, the REE-binding protein comprises an EF-hand domain. In some embodiments, the REE is then recovered from the sample. In some embodiments, the methods further comprise purifying an REE from the sample.

Further aspects of the disclosure relate to host cells comprising a heterologous gene expressing a rare earth element binding protein. In some embodiments, the rare earth element binding protein in the host cells comprises an EF-hand domain. In some embodiments, the REE binding protein does not comprise SEQ ID NO: 1.

Further aspects of the disclosure relate to kits comprising any of the host cells disclosed in this application and instructions for extraction of a rare earth element. In some embodiments, the REE binding protein comprises an EF-hand domain.

In some embodiments, the EF-hand domain comprises the sequence: X₁X₂X₃X₄X₅X₆X₇X₈X₉X₁₀X₁₁X₁₂ (SEQ ID NO: 1275). In some embodiments, X denotes any amino acid. In some embodiments, X₁ is D or K. In some embodiments, X₂ is A, D, E, F, G, H, I, K, L, P, Q, R, S, T, V, or Y. In some embodiments, X₃ is D, E, N, Q, S, or T. In some embodiments, X₄ is A, D, E, G, H, K, L, N, Q, R, S, or T. In some embodiments, X₅ is D, N, S, or T. In some embodiments, X₆ is A, E, G, K, N, Q, R, or S. In some embodiments, X₇ is A, D, E, F, G, H, I, K, L, M, N, Q, R, S, T, V, or Y. In some embodiments, X₈ is A, F, I, L, M, or V. In some embodiments, X₉ is A, D, E, G, L, N, Q, S, T, or V. In some embodiments, X₁₀ is A, D, E, F, G, H, I, K, L, M, N, P, Q, R, S, T, V, or Y. In some embodiments, X₁₁ is A, D, E, F, G, I, K, L, M, N, Q, R, S, T, V, or Y. In some embodiments, X₁₂ is A, D, E, G, I, K, L, Q, R, S, T, or V. In some embodiments, the EF-hand domain additionally comprises X₁₃. In some embodiments, X₁₃ is A, E, F, G, H, I, L, M, Q, R, S, T, V, W, or Y.

In some embodiments, the EF-hand domain comprises the sequence: DX₁X₂X₃X₄X₅X₆X₇X₈X₉X₁₀X₁₁ (SEQ ID NO: 1276). In some embodiments, X₁ is A, D, F, G, I, K, L, P, Q, R, S, T, V, or Y. In some embodiments, X₂ is D, N, Q, or S. In some embodiments, X₃ is A, D, G, H, K, N, Q, R, or S. In some embodiments, X₄ is D, N, or S. In some embodiments, X₅ is A, E, G, K, N, Q, or R. In some embodiments, X₆ is A, D, E, F, H, I, K, L, M, N, Q, R, S, T, V, or Y. In some embodiments, X₇ is A, F, I, L, or V. In some embodiments, X₈ is D, E, G, N, S, T, or V. In some embodiments, X₉ is A, D, E, F, G, H, I, K, L, M, N, P, Q, R, S, T, V, or Y. In some embodiments, X₁₀ is A, D, E, F, G, I, K, L, N, Q, R, S, T, V, or Y. In some embodiments, X₁₁ is A, D, E, G, L, Q, R, S, T, or V. In some embodiments, the EF-hand domain additionally comprises X₁₂. In some embodiments, X₁₂ is A, E, F, G, H, I, L, M, Q, R, S, V, W, or Y.

In some embodiments, the EF-hand domain comprises the sequence: DX₁X₂X₃X₄X₅X₆X₇X₈X₉X₁₀X₁₁ (SEQ ID NO: 1277). In some embodiments, X₁ is A, D, F, G, I, K, L, Q, R, S, T, V, or Y. In some embodiments, X₂ is D, N, Q, or S. In some embodiments, X₃ is A, D, G, H, K, N, Q, R, or S. In some embodiments, X₄ is D, N, or S. In some embodiments, X₅ is A, E, G, K, N, Q, or R. In some embodiments, X₆ is A, D, E, F, H, I, K, L, M, N, Q, R, S, T, V, or Y. In some embodiments, X₇ is A, F, I, L, or V. In some embodiments, X₈ is D, E, G, N, S, T, or V. In some embodiments, X₉ is A, D, E, F, G, H, I, K, L, M, N, P, Q, R, S, T, V, or Y. In some embodiments, X₁₀ is A, D, E, F, G, I, K, L, N, Q, R, S, T, V, or Y. In some embodiments, X₁₁ is A, D, E, G, L, Q, R, S, T, or V. In some embodiments, the EF-hand domain additionally comprises X₁₂. In some embodiments, X₁₂ is A, E, F, G, H, I, L, M, Q, R, S, V, W, or Y.

In some embodiments, the EF-hand domain comprises no more than two amino acid substitutions relative to an EF-hand domain sequence in Table 2, corresponding to SEQ ID NOs: 300-977. In some embodiments, the EF-hand domain comprises a sequence selected from the EF-hand domain sequences in Table 2, corresponding to SEQ ID NOs: 300-977. In some embodiments, the EF-hand domain comprises a sequence selected from SEQ ID NOs: 304-715. In some embodiments, the EF-hand domain comprises a sequence selected from SEQ ID NOs: 304-473.

In some embodiments, the REE binding protein comprises at least two EF-hand domain sequences. In some embodiments, the REE binding protein comprises at least three EF-hand domain sequences. In some embodiments, the REE binding protein comprises at least four EF-hand domain sequences. In some embodiments, at least two EF-hand domain sequences are different.

In some embodiments, the REE binding protein is a lanthanide binding protein. In some embodiments, the REE binding protein is a yttrium binding protein. In some embodiments, the REE binding protein is a scandium binding protein. In some embodiments, the REE binding protein is a lanthanide binding protein and a yttrium binding protein. In some embodiments, the REE binding protein is a lanthanide binding protein and a scandium binding protein. In some embodiments, the REE binding protein is a yttrium binding protein and a scandium binding protein. In some embodiments, the REE binding protein is a lanthanide binding protein, a yttrium binding protein, and a scandium binding protein.

In some embodiments, the REE binding protein has at least 10%, 20%, 30%, 40%, 50%, 60%, 70%, 80%, 90%, or 100% higher lanthanide binding affinity relative to a control protein. In some embodiments, the control protein comprises SEQ ID NO: 2. In some embodiments, the control protein comprises SEQ ID NO: 3. In some embodiments, the binding affinity is determined using a dialysis assay. In some embodiments, the lanthanide is selected from the group consisting of cerium (Ce), dysprosium (Dy), erbium (Er), europium (Eu), gadolinium (Gd), holmium (Ho), lanthanum (La), lutetium (Lu), neodymium (Nd), praseodymium (Pr), promethium (Pm), samarium (Sm), terbium (Tb), thulium (Tm), ytterbium (Yb), and a combination thereof.

In some embodiments, the REE binding protein has at least 10%, 20%, 30%, 40%, 50%, 60%, 70%, 80%, 90%, or 100% lower iron binding affinity relative to a control protein. In some embodiments, the control protein is TRFE_HUMAN2 (SEQ ID NO: 298). In some embodiments, the control protein is TRFE_BOVIN2 (SEQ ID NO: 299). In some embodiments, the binding affinity is determined using a dialysis assay.

In some embodiments, the REE binding protein is expressed as a heterologous gene in a host cell. In some embodiments, the REE binding protein is secreted from the host cell. In some embodiments, the REE-binding protein comprises a sequence that is at least 90% identical to any one of SEQ ID NOs: 4-71. In some embodiments, the REE-binding protein comprises any one of SEQ ID NOs: 4-71. In some embodiments, the REE-binding protein comprises at least 90% identity to the portion of any one of SEQ ID NOs: 4-71 outside of the EF hand domains within the sequence, and comprises no more than two amino acid substitutions relative to the EF-hand domain(s) within the sequence.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used in this disclosure is for the purpose of description and should not be regarded as limiting. The use of “including,” “comprising,” or “having,” “containing,” “involving,” and variations of thereof in this disclosure, is meant to encompass the items listed thereafter and equivalents thereof as well as additional items.

### BRIEF DESCRIPTION OF DRAWINGS

The accompanying drawings are not intended to be drawn to scale. The drawings are illustrative only and are not required for enablement of the disclosure. For purposes of clarity, not every component may be labeled in every drawing. In the drawings:

FIG. 1 shows the total number of EF-hand domains in putative lanthanide-binding proteins from four different samples (Sample 1, Sample 2 (Tailings), Sample 3, and Sample 4). From left to right for each number, the sources correspond to Sample 1, Sample 2 (Tailings), Sample 3, and Sample 4. Sample 2 (Tailings) refers to a sample with a high level of dysprosium and other lanthanides.

FIG. 2 is a schematic showing an overview of metal-binding affinity characterization of candidate lanthanide-binding proteins. Cells were transformed with plasmids encoding candidate lanthanide-binding proteins that comprise one or more EF-hand domains. The candidate proteins were expressed, purified and analyzed using a Xylenol Orange (XO) based terbium competition assay in a primary screen and in dialysis and chromatography assays in a secondary screen. The binding selectivity of top hits for terbium as compared to iron was also characterized.

FIG. 3 includes schematics depicting a primary screen to identify lanthanide-binding proteins (LBP). An illustration of the XO competition assay is shown on the left. A pie chart showing the breakdown of proteins measured in the primary screen is provided on the right.

FIG. 4 includes two graphs showing identification of proteins that bind the lanthanide metal terbium using the primary screen depicted in FIG. 3. The top graph depicts data from the XO competition assays for all candidate lanthanide-binding proteins (which are referred to as “Strains”). The bottom graph depicts representative hits discovered by the primary screen. In the bottom graph, the strains in Batch 1 included: t201150, t201097, t200855, t201015, t200947, t200901, t200881, t201161, t201209, t200928, t200955, t200879, t200862, t200769, t200987, t200701, t201213, t200733, and t200949; the strains in Batch 2 included: t201777, t201380, t201734, t201629, t201856, t201782, t201817, t201697, t201609, t201647, t201742, t201599, t201897, t201314, t201750, t201859, t201631, t201795, and t201779; the strains in Batch 3 included: t201331, t201245, t201369, t201380, t201726, t201734, t201463, t201856, t201277, t201539, t201413, t201595, t201587, t201406, t201425, t201285, t201547, t201424, t201217, and t201253; and the strains in Batch 4 included: t200581, t200773, t200818, t200603, t200741, t200574, t200830, t200594, t200701, t200586, t200753, t200666, t200795, t200784, t200791, t200770, t200832, t200729, and t200628.

FIG. 5 includes data showing the terbium-binding affinity of candidate lanthanide-binding proteins. The left graph shows results from a dialysis-plate assay for all proteins (294 candidate lanthanide-binding proteins plus three positive controls). The right graph shows the fluorescence signal normalized by protein sample concentration, considering only strains (proteins) with protein concentrations greater than or equal to 20 μM. The positive controls are indicated by three arrows. The two lower-affinity terbium-binder controls are designed lanthanide-binding proteins and the single higher-affinity control strain is the lanthanide-binding protein, LanM, from a lanthanide-utilizing bacterium.

FIG. 6 includes graphs showing the affinity of lanthanide binding proteins for dysprosium and iron. Dialysis-plate assay results for the 23 best-binder proteins from the terbium experiments after overnight incubation with 2 μM dysprosium (left) or 2 μM iron (right) are shown. The graph on the left shows the amount of bound dysprosium quantified by Delfia fluorescence assay by measuring emission at 572 nm following excitation at 320 nm. The intensity signal is normalized to protein concentration, and strains are ranked in order from lowest (left-hand side of plot) to highest (right-hand side of plot) affinity for dysprosium. The positive controls (arrows) are the same as in the terbium experiments. The graph on the right shows iron-binding quantified through Fe-ferrozine complexation by measuring the absorbance at 560 nm (Abs560). Proteins are ranked according to their protein concentration-normalized iron-binding abilities. In these assays, high iron-binding affinity bovine and human transferrin proteins (far right-hand side, double arrows) are used as positive controls.

### DETAILED DESCRIPTION

Rare earth elements (REEs) have diverse uses and are critical to the production of catalysts, alloys, high-performance magnets, glasses, and electronics. Despite the abundance of REEs in the earth's crust, however, mineable ore deposits are rare. This disclosure is premised, in part, on the identification of multiple REE-binding proteins. REE-binding proteins described in this disclosure may be leveraged to recover REEs from samples, including recovery of REEs from machining waste and from electronic waste. Accordingly, provided in this disclosure are newly-identified REE-binding proteins, host cells comprising the REE-binding proteins, and methods of using the REE-binding proteins to recover REEs.

REEs comprise a group of metals including lanthanides, yttrium (Y), and scandium (Sc). The lanthanides (or lanthanoids) are elements with atomic numbers 57 through 71 (e.g., lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu), respectively). The metallic radius of the lanthanides are as follows: La: 162 pm; Ce: 181.8 pm; Pr: 182.4 pm; Nd: 181.4 pm; Pm: 183.4 pm; Sm: 180.4 pm; Eu: 208.4 pm; Gd: 180.4 pm; Tb: 177.3 pm; Dy: 178.1 pm; Ho: 176.2 pm; Er: 176.1 pm; Tm: 175.9 pm; Yb: 193.3 pm; and Lu: 173.8 pm. Yttrium is a metal with atomic number 39 and an atomic radius of 180 pm. Scandium is a metal with atomic number 21 and an atomic radius of 162 pm. The ionic radius of the rare earth elements are as follows: La: 1.045 Å; Ce: 1.01 Å; Pr: 0.997 Å; Nd: 0.983 Å; Pm: 0.97 Å; Sm: 0.958 Å; Eu: 0.947 Å; Gd: 0.938 Å; Tb: 0.923 Å; Dy: 0.912 Å; Ho: 0.901 Å; Er: 0.89 Å; Tm: 0.88 Å; Yb: 0.868 Å; Lu: 0.861 Å; Sc: 0.745 Å; and Y: 0.9 Å. The crystal radius value of each rare earth element is also known. See, e.g., Shannon, Acta Cryst. (1976). A32, 751-767.

### Rare Earth Element (REE)-Binding Proteins

Aspects of the present disclosure provide rare earth element (REE)-binding proteins, which may be useful, for example, in the recovery of a REE from a sample. In some embodiments, the REE-binding proteins of the present disclosure comprise an EF-hand domain. EF-hand domains are motifs involved in binding to metals and are commonly found in calcium-binding proteins. EF-hand domains are described further in Cotruvo et al. (2018) J. Am. Chem. Soc. 140(44):15056-15061, incorporated by reference in this application in its entirety. Without wishing to be bound by any theory, the EF-hand binding domain is relatively compact, suggesting that the ratio of protein to lanthanide will be higher than for other metal-binding domains known in the art. Accordingly, EF-hand domains may be useful as a potential filtering agent for metals of interest. In some embodiments, a metal of interest is a REE. In some embodiments, a metal of interest is a transition metal. In some embodiments, a metal of interest is a lanthanide.

As discussed in Example 1, a large number of proteins with multiple EF-hand domains were identified in this disclosure. The presence of multiple EF-hand domains in individual proteins suggests that these proteins may be able to bind multiple lanthanide atoms per protein. Samples that were extracted from lanthanide-rich tailing ponds comprised numerous proteins with at least two EF-hand domains per protein.

In some embodiments, an EF-hand domain comprises a contiguous sequence of at least twelve amino acids with the sequence X₁X₂X₃X₄X₅X₆X₁X₈X₉X₁₀X₁₁X₁₂ (SEQ ID NO: 1275), wherein X denotes any amino acid, X₁ is D or K; X₂ is A, D, E, F, G, H, I, K, L, P, Q, R, S, T, V, or Y; X₃ is D, E, N, Q, S, or T; X₄ is A, D, E, G, H, K, L, N, Q, R, S, or T; X₅ is D, N, S, or T; X₆ is A, E, G, K, N, Q, R, or S; X₇ is A, D, E, F, G, H, I, K, L, M, N, Q, R, S, T, V, or Y; X₈ is A, F, I, L, M, or V; X₉ is A, D, E, G, L, N, Q, S, T, or V; X₁₀ is A, D, E, F, G, H, I, K, L, M, N, P, Q, R, S, T, V, or Y; X₁₁ is A, D, E, F, G, I, K, L, M, N, Q, R, S, T, V, or Y; and X₁₂ is A, D, E, G, I, K, L, Q, R, S, T, or V.

In some embodiments, an EF-hand domain comprises a contiguous sequence of at least thirteen amino acids with the sequence X₁X₂X₃X₄X₅X₆X₇X₈X₉X₁₀X₁₁X₁₂X₁₃ (SEQ ID NO: 1275), wherein X denotes any amino acid, X₁ is D or K; X₂ is A, D, E, F, G, H, I, K, L, P, Q, R, S, T, V, or Y; X₃ is D, E, N, Q, S, or T; X₄ is A, D, E, G, H, K, L, N, Q, R, S, or T; X₅ is D, N, S, or T; X₆ is A, E, G, K, N, Q, R, or S; X₇ is A, D, E, F, G, H, I, K, L, M, N, Q, R, S, T, V, or Y; X₈ is A, F, I, L, M, or V; X₉ is A, D, E, G, L, N, Q, S, T, or V; X₁₀ is A, D, E, F, G, H, I, K, L, M, N, P, Q, R, S, T, V, or Y; X₁₁ is A, D, E, F, G, I, K, L, M, N, Q, R, S, T, V, or Y; and X₁₂ is A, D, E, G, I, K, L, Q, R, S, T, or V; and X₁₃ is A, E, F, G, H, I, L, M, Q, R, S, T, V, W, or Y.

In some embodiments, an EF-hand domain comprises a contiguous sequence of at least twelve amino acids with the sequence DX₁X₂X₃X₄X₅X₆X₇X₈X₉X₁₀X₁₁ (SEQ ID NO: 1276), wherein X denotes any amino acid, X₁ is A, D, F, G, I, K, L, P, Q, R, S, T, V, or Y; X₂ is D, N, Q, or S; X₃ is A, D, G, H, K, N, Q, R, or S; X₄ is D, N, or S; X₅ is A, E, G, K, N, Q, or R; X₆ is A, D, E, F, H, I, K, L, M, N, Q, R, S, T, V, or Y; X₇ is A, F, I, L, or V; X₈ is D, E, G, N, S, T, or V; X₉ is A, D, E, F, G, H, I, K, L, M, N, P, Q, R, S, T, V, or Y; X₁₀ is A, D, E, F, G, I, K, L, N, Q, R, S, T, V, or Y; and X₁₁ is A, D, E, G, L, Q, R, S, T, or V.

In some embodiments, an EF-hand domain comprises a contiguous sequence of at least thirteen amino acids with the sequence DX₁X₂X₃X₄X₅X₆X₇X₈X₉X₁₀X₁₁X₁₂ (SEQ ID NO: 1276), wherein X denotes any amino acid, X₁ is A, D, F, G, I, K, L, P, Q, R, S, T, V, or Y; X₂ is D, N, Q, or S; X₃ is A, D, G, H, K, N, Q, R, or S; X₄ is D, N, or S; X₅ is A, E, G, K, N, Q, or R; X₆ is A, D, E, F, H, I, K, L, M, N, Q, R, S, T, V, or Y; X₇ is A, F, I, L, or V; X₈ is D, E, G, N, S, T, or V; X₉ is A, D, E, F, G, H, I, K, L, M, N, P, Q, R, S, T, V, or Y; X₁₀ is A, D, E, F, G, I, K, L, N, Q, R, S, T, V, or Y; X₁₁ is A, D, E, G, L, Q, R, S, T, or V; and X₁₂ is A, E, F, G, H, I, L, M, Q, R, S, V, W, Y.

In some embodiments, an EF-hand domain comprises a contiguous sequence of at least twelve amino acids with the sequence DX₁X₂X₃X₄X₅X₆X₇X₈X₉X₁₀X₁₁ (SEQ ID NO: 1277), wherein X denotes any amino acid, X₁ is A, D, F, G, I, K, L, Q, R, S, T, V, or Y; X₂ is D, N, Q, or S; X₃ is A, D, G, H, K, N, Q, R, or S; X₄ is D, N, or S; X₅ cis A, E, G, K, N, Q, or R; X₆ is A, D, E, F, H, I, K, L, M, N, Q, R, S, T, V, or Y; X₇ is A, F, I, L, or V; X₈ is D, E, G, N, S, T, or V; X₉ is A, D, E, F, G, H, I, K, L, M, N, P, Q, R, S, T, V, or Y; X₁₀ is A, D, E, F, G, I, K, L, N, Q, R, S, T, V, or Y; and X₁₁ is A, D, E, G, L, Q, R, S, T, or V.

In some embodiments, an EF-hand domain comprises a contiguous sequence of at least twelve amino acids with the sequence DX₁X₂X₃X₄X₅X₆X₇X₈X₉X₁₀X₁₁X₁₂ (SEQ ID NO: 1277), wherein X denotes any amino acid, X₁ is A, D, F, G, I, K, L, Q, R, S, T, V, or Y; X₂ is D, N, Q, or S; X₃ is A, D, G, H, K, N, Q, R, or S; X₄ is D, N, or S; X₅ is A, E, G, K, N, Q, or R; X₆ is A, D, E, F, H, I, K, L, M, N, Q, R, S, T, V, or Y; X₇ is A, F, I, L, or V; X₈ is D, E, G, N, S, T, or V; X₉ is A, D, E, F, G, H, I, K, L, M, N, P, Q, R, S, T, V, or Y; X₁₀ is A, D, E, F, G, I, K, L, N, Q, R, S, T, V, or Y; X₁₁ is A, D, E, G, L, Q, R, S, T, or V; and X₁₂ is A, E, F, G, H, I, L, M, Q, R, S, V, W, or Y.

In some instances, the EF-hand domain comprises a contiguous stretch of at least 12, 13, 14, 15, 16, 17, 18, 19, 20, 21, 22, 23, 24, 25, 26, 27, 28, 29, or at least 30 amino acids.

In some instances, the EF-hand domain comprises a helix-loop-helix structure. In some instances, the loop in the helix-loop-helix structure comprises any of the EF-hand domain sequences described in this disclosure. In some embodiments, the loop in the helix-loop-helix structure binds one or more metal ions. In some embodiments, an EF-hand domain has a tertiary structure that is similar to the tertiary structure of an EF-hand domain that binds Ca2+.

The EF-hand domain may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to a EF-hand domain sequence in Table 2 (e.g., a sequence selected from SEQ ID NOs: 300-977, 300-715, 304-473, or 304-715).

An REE-binding protein may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 EF-hand domains.

In some instances, in an REE-binding protein that comprises more than 1 EF-hand domain, at least at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 EF-hand domains in the REE-binding protein are different.

In some instances, in an REE-binding protein that comprises more than 1 EF-hand domain, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 EF-hand domains in the REE-binding protein are the same.

REE-binding proteins may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to a sequence (e.g., nucleic acid or amino acid sequence) in Table 2 or to a sequence selected from SEQ ID NOs: 1-297, 1-179, 4-71, 4-179, 978-1274, 981-1274, 981-1156, or 981-1048.

An REE-binding protein may be capable of binding one or more REEs. For example, the REE may be capable of binding lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), yttrium (Y), scandium (Sc) or a combination thereof. In some instances, the REE-binding protein is a lanthanum (La)-binding protein, a cerium (Ce)-binding protein, a praseodymium (Pr)-binding protein, a neodymium (Nd)-binding protein, a promethium (Pm)-binding protein, a samarium (Sm)-binding protein, an europium (Eu)-binding protein, a gadolinium (Gd)-binding protein, a terbium (Tb)-binding protein, a dysprosium (Dy), a holmium (Ho), an erbium (Er), a thulium (Tm), an ytterbium (Yb), a lutetium (Lu)-binding protein, an yttrium (Y)-binding protein, a scandium (Sc)-binding protein, or a combination thereof. In some instances, the REE-binding protein is a lanthanide-binding protein.

Without being bound by a particular theory, metal ion valence and size of the metal ion may influence EF-hand metal binding affinity. For example, scandium and yttrium ions are usually trivalent cations in solution, just like the lanthanides terbium and dysprosium. The ionic radius of yttrium (104 pm) is similar to that of terbium (106 pm) and dysprosium (105 pm), while scandium is somewhat smaller (89 pm). Without being by bound a particular theory, given the ionic radii, proteins that bind lanthanides with high affinity will have likely have high affinity for yttrium and may also bind scandium.

An REE-binding protein may be capable of binding at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 REE ions. In some instances, an REE-binding protein is capable of binding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 of the same type of REE ions. In some instances, an REE-binding protein is capable of binding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 different types of REE ions.

It should be appreciated that binding affinity of a REE-binding protein can be measured by any means known to one of ordinary skill in the art. In some embodiments, the binding affinity of a REE-binding protein may be measured using a metal competition assay. Non-limiting examples of assays to measure the affinity of a REE-binding protein for a particular metal include a Xylenol Orange (XO) competition assay, a dialysis assay, absorption spectroscopy, fluorescence spectroscopy, x-ray spectroscopy, capillary electrophoresis, circular dichroism, and isothermal titration calorimetry. For example, an REE-binding dialysis-based assay may be used to measure the REE-binding affinity of a particular REE-binding protein. In some embodiments, the REE is a lanthanide. In some embodiments, the REE is dysprosium or terbium. In some instances, the affinity of a REE-binding protein to a particular metal is normalized to the concentration of protein used. The iron-binding affinity of an REE-binding protein may be measured using any method known in the art, including a Ferrozine chromogenic assay. See also, e.g., Farrell et al., Protoplasma 1990, 159, 157-167; Rasmussen et al., Inorg. Biochem. 2003, 95, 113-123; Hebenstreit and Ferreira, Allergy 2005, 60, 1208-1211; Saboury et al., Therm. Anal. Calorim. 2006, 83, 175-179; and the Examples below for methods of measuring the affinity of a metal ion for a protein.

In some instances, the affinity of a metal ion M for protein ligand P is defined as the dissociation constant K_{D}=[M][P]/[MP]. In some instances, the K_{D} is less than 1×10⁻⁵M, less than 5×10⁻⁶M, less than 1×10⁻⁶M, less than 5×10⁻⁷M, less than 1×10⁻⁷M, less than 5×10⁻⁸M, less than 1×10⁻⁸M, less than 5×10⁻⁹M, less than 1×10⁻⁹M, less than 5×10⁻¹⁰ M, less than 1×10⁻¹⁰ M, less than 5×10⁻¹¹M, less than 1×10⁻¹¹M, less than 5×10⁻¹²M, less than 1×10⁻¹²M, less than 5×10⁻¹³M, less than 1×10⁻¹³M, less than 1×10⁻¹⁴M, less than 5×10⁻¹⁴M, less than 5×10⁻¹⁵M, less than 1×10⁻¹⁵M, less than 5×10⁻¹⁶M, less than 1×10⁻¹⁶ M, less than 5×10⁻¹⁷M, less than 1×10⁻¹⁷M, less than 5×10⁻¹⁸M, less than 1×10⁻¹⁸M, less than 5×10⁻¹⁹M, less than 1×10⁻¹⁹M, less than 5×10⁻²⁰ M, or less than 1×10⁻²⁰ M, including any values in between.

The affinity of an REE-binding protein for different metals may also be compared. The affinity of an REE-binding protein for an REE may be at least 1.5-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 30-fold, at least 40-fold, at least 50-fold, at least 60-fold, at least 70-fold, at least 80-fold, at least 90-fold, at least 100-fold, at least 200-fold, at least 300-fold, at least 400-fold, at least 500-fold, at least 600-fold, at least 700-fold, at least 800-fold, at least 900-fold, or at least 1,000-fold, including any values in between, greater than the affinity of the REE-binding protein for another metal (e.g., a different REE, iron, or calcium).

The affinity of an REE-binding protein for a particular metal may also be compared to the affinity of a control protein for the same metal. Non-limiting examples of control proteins can include, for example, one or more of SEQ ID NO: 1, SEQ ID NO: 2, SEQ ID NO: 3, SEQ ID NO: 298 and SEQ ID NO: 299. For example, the affinity of an REE-binding protein for a metal may be at least 1.5-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 30-fold, at least 40-fold, at least 50-fold, at least 60-fold, at least 70-fold, at least 80-fold, at least 90-fold, at least 100-fold, at least 200-fold, at least 300-fold, at least 400-fold, at least 500-fold, at least 600-fold, at least 700-fold, at least 800-fold, at least 900-fold, or at least 1,000-fold greater than the affinity of a control protein for the same metal.

### Variants

Variants of sequences described in the present disclosure (e.g., EF-hand domains and/or REE-binding proteins) are also encompassed by the present disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleic acid and/or amino acid sequence identity with a reference sequence, including all values in between.

In some embodiments, variants include proteins in which the EF-hand domain(s) within a protein are 100% conserved, but other portions of the protein may be less conserved. For example, in some embodiments, a variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% nucleic acid and/or amino acid sequence identity with the portion of a reference sequence that excludes the EF-hand domain(s), including all values in between, while sharing 100% sequence identity with the EF-hand domain(s) of the reference sequence.

Unless otherwise noted, the term “sequence identity,” as known in the art, refers to a relationship between the sequences of two polypeptides or polynucleotides, as determined by sequence comparison (alignment). In some embodiments, sequence identity is determined across the entire length of a sequence (e.g., EF-hand domains and/or REE-binding proteins). In some embodiments, sequence identity is determined over a region (e.g., a stretch of amino acids or nucleic acids, e.g., the sequence spanning an active site) of a sequence (e.g., EF-hand domains and/or REE-binding proteins) or a region that does not span the active site (e.g., the portion of a nucleic acid or protein sequence that excludes EF-hand domains).

Identity can also refer to the degree of sequence relatedness between two sequences as determined by the number of matches between strings of two or more residues (e.g., nucleic acid or amino acid residues). Identity measures the percent of identical matches between two or more sequences with gap alignments (if any) addressed by a particular mathematical model or computer program (e.g., algorithms).

Identity of related polypeptides or nucleic acid sequences can be readily calculated by any of the methods known to one of ordinary skill in the art. The “percent identity” of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3 to obtain amino acid sequences homologous to the proteins described in this disclosure. Where gaps exist between two sequences, Gapped BLAST® can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotide and dividing by the length of one of the nucleic acids.

For multiple sequence alignments, computer programs including Clustal Omega (Sievers et al., Mol Syst Biol. 2011 Oct. 11; 7:539) may be used.

In preferred embodiments, a sequence, including a nucleic acid or amino acid sequence, is found to have a specified percent identity to a reference sequence, such as a sequence disclosed in this application and/or recited in the claims when sequence identity is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993 (e.g., BLAST®, NBLAST®, XBLAST® or Gapped BLAST® programs, using default parameters of the respective programs).

In some embodiments, a sequence, including a nucleic acid or amino acid sequence, is found to have a specified percent identity to a reference sequence, such as a sequence disclosed in this application and/or recited in the claims when sequence identity is determined using the Smith-Waterman algorithm (Smith, T. F. & Waterman, M. S. (1981) “Identification of common molecular subsequences.” J. Mol. Biol. 147:195-197) or the Needleman-Wunsch algorithm (Needleman, S. B. & Wunsch, C. D. (1970) “A general method applicable to the search for similarities in the amino acid sequences of two proteins.” J. Mol. Biol. 48:443-453) using default parameters.

In some embodiments, a sequence, including a nucleic acid or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA) using default parameters.

In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct. 11; 7:539) using default parameters.

As used in this disclosure, and as would be understood by one of ordinary skill in the art, a residue (such as a nucleic acid residue or an amino acid residue) in sequence “X” is referred to as corresponding to a position or residue (such as a nucleic acid residue or an amino acid residue) “Z” in a different sequence “Y” when the residue in sequence “X” is at the counterpart position of “Z” in sequence “Y” when sequences X and Y are aligned using amino acid sequence alignment tools known in the art, such as, for example, Clustal Omega or BLAST®.

As used in this disclosure, variant sequences may be homologous sequences. As used in this disclosure, homologous sequences refers to sequences (e.g., nucleic acid or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity, including all values in between). Homologous sequences include but are not limited to paralogous or orthologous sequences. Paralogous sequences arise from duplication of a gene within a genome of a species, while orthologous sequences diverge after a speciation event.

In some embodiments, a peptide or polypeptide variant (e.g., EF-hand domain or REE-binding protein variant) comprises a domain that shares a secondary structure (e.g., alpha helix, beta sheet) with a reference peptide or polypeptide (e.g., a reference EF-hand domain or REE-binding protein). In some embodiments, a peptide or polypeptide variant (e.g., EF-hand domain or REE-binding protein variant) shares a tertiary structure with a reference peptide or polypeptide (e.g., a reference EF-hand domain or REE-binding protein). As a non-limiting example, a variant peptide or polypeptide (e.g., EF-hand domain or REE-binding protein) may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets), or have the same tertiary structure as a reference EF-hand domain or REE-binding protein. For example, a loop may be located between two alpha helices, or between two beta sheets. Homology modeling may be used to compare two or more tertiary structures.

Any suitable method, including circular permutation (Yu and Lutz, Trends Biotechnol. 2011 January; 29(1):18-25), may be used to produce such variants. In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%, including all values in between) as determined by linear sequence alignment methods (e.g., Clustal Omega or BLAST). Topological analysis of the two proteins, however, may reveal that the tertiary structure of the two polypeptides is similar. Without being bound by a particular theory, a variant polypeptide created through circular permutation of a reference polypeptide and with a tertiary structure that is similar to the tertiary structure of the reference polypeptide can share similar functional characteristics (e.g., enzymatic activity, enzyme kinetics, substrate specificity or product specificity). In some instances, circular permutation may alter the secondary structure, tertiary structure or quaternary structure and produce an enzyme with different functional characteristics (e.g., increased or decreased enzymatic activity, different substrate specificity, or different product specificity). See, e.g., Yu and Lutz, Trends Biotechnol. 2011 January; 29(1):18-25.

It should be appreciated that in a protein that has undergone circular permutation, the linear amino acid sequence of the protein would differ from a reference protein that has not undergone circular permutation. However, one of ordinary skill in the art would be able to readily determine which residues in the protein that has undergone circular permutation correspond to residues in the reference protein that has not undergone circular permutation by, for example, aligning the sequences and detecting conserved motifs, and/or by comparing the structures or predicted structures of the proteins, e.g., by homology modeling. Variants described in this application include circularly permutated variants of sequences described in this application.

In some embodiments, an algorithm that determines the percent identity between a sequence of interest and a reference sequence described in this application accounts for the presence of circular permutation between the sequences. The presence of circular permutation may be detected using any method known in the art, including, for example, RASPODOM (Weiner et al., Bioinformatics. 2005 Apr. 1; 21(7):932-7). In some embodiments, the presence of circulation permutation is corrected for (e.g., the domains in at least one sequence are rearranged) prior to calculation of the percent identity between a sequence of interest and a sequence described in this application. The claims of this application should be understood to encompass sequences for which percent identity to a reference sequence is calculated after taking into account potential circular permutation of the sequence.

Functional variants of the recombinant REE-binding proteins described in this disclosure are also encompassed by the present disclosure. For example, functional variants may bind one or more of the same substrates or produce one or more of the same products. Functional variants may be identified using any method known in the art. For example, the algorithm of Karlin and Altschul Proc. Natl. Acad. Sci. USA 87:2264-68, 1990 described above may be used to identify homologous proteins with known functions.

Putative functional variants may also be identified by searching for polypeptides with functionally annotated domains. Databases including Pfam (Sonnhammer et al., Proteins. 1997 July; 28(3):405-20) may be used to identify polypeptides with a particular domain.

Homology modeling may also be used to identify amino acid residues that are amenable to mutation without affecting function. A non-limiting example of such a method may include use of position-specific scoring matrix (PSSM) and an energy minimization protocol.

Position-specific scoring matrix (PSSM) uses a position weight matrix to identify consensus sequences (e.g., motifs). PSSM can be conducted on nucleic acid or amino acid sequences. Sequences are aligned, and the method takes into account the observed frequency of a particular residue (e.g., an amino acid or a nucleotide) at a particular position and the number of sequences analyzed. See, e.g., Stormo et al., Nucleic Acids Res. 1982 May 11; 10(9):2997-3011. The likelihood of observing a particular residue at a given position can be calculated. Without being bound by a particular theory, positions in sequences with high variability may be amenable to mutation (e.g., PSSM score ≥0) to produce functional homologs.

PSSM may be paired with calculation of a Rosetta energy function, which determines the difference between the wild-type and the single-point mutant. The Rosetta energy function calculates this difference as (ΔΔG_{calc}). With the Rosetta function, the bonding interactions between a mutated residue and the surrounding atoms are used to determine whether a mutation increases or decreases protein stability. For example, a mutation that is designated as favorable by the PSSM score (e.g. PSSM score ≥0), can then be analyzed using the Rosetta energy function to determine the potential impact of the mutation on protein stability. Without being bound by a particular theory, potentially stabilizing mutations are desirable for protein engineering (e.g., production of functional homologs). In some embodiments, a potentially stabilizing mutation has a ΔΔG_{calc} value of less than −0.1 (e.g., less than −0.2, less than −0.3, less than −0.35, less than −0.4, less than −0.45, less than −0.5, less than −0.55, less than −0.6, less than −0.65, less than −0.7, less than −0.75, less than −0.8, less than −0.85, less than −0.9, less than −0.95, or less than −1.0) Rosetta energy units (R.e.u.). See, e.g., Goldenzweig et al., Mol Cell. 2016 Jul. 21; 63(2):337-346. Doi: 10.1016/j.molcel.2016.06.012.

In some embodiments, an REE-binding protein coding sequence comprises a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 positions corresponding to a reference (e.g., REE-binding protein) coding sequence. In some embodiments, an REE-binding protein coding sequence comprises a mutation at no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 positions corresponding to a reference (e.g., REE-binding protein) coding sequence. In some embodiments, an REE-binding protein coding sequence comprises a mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more codons of the coding sequence relative to a reference (e.g., REE-binding protein) coding sequence.

In some embodiments, an EF-hand domain sequence comprises a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 positions corresponding to a reference EF-hand domain coding sequence. In some embodiments, an EF-hand domain sequence comprises a mutation at no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 positions corresponding to a reference EF-hand domain coding sequence. In some embodiments, an EF-hand domain coding sequence comprises a mutation in 1, 2, 3, 4, 5, 6, 7, 8, 9, or 10 codons of the coding sequence relative to a reference EF-hand domain coding sequence. In some embodiments, a reference EF-hand domain coding sequence is selected from a EF-hand domain sequence in Table 2 (e.g., SEQ ID NOs: 300-977).

As will be understood by one of ordinary skill in the art, a mutation within a codon may or may not change the amino acid that is encoded by the codon due to degeneracy of the genetic code. In some embodiments, the one or more mutations in the coding sequence do not alter the amino acid sequence of the coding sequence (e.g., EF-hand domain or REE-binding protein) relative to the amino acid sequence of a reference polypeptide (e.g., a reference EF-hand domain or REE-binding protein).

In some embodiments, the one or more mutations in a recombinant EF-hand domain or REE-binding protein sequence alters the amino acid sequence of the peptide or polypeptide relative to the amino acid sequence of a reference polypeptide (e.g., EF-hand domain or REE-binding protein). In some embodiments, the one or more mutations alters the amino acid sequence of the recombinant REE-binding protein or EF-hand domain relative to the amino acid sequence of a reference REE-binding protein or EF-hand domain and alters (enhances or reduces) an activity of the REE-binding protein or EF-hand domain relative to the reference REE-binding protein or EF-hand domain.

The activity (e.g., specific activity) of any of the recombinant REE-binding proteins or EF-hand domains described in this disclosure may be measured using methods known to one of ordinary skill in the art. As a non-limiting example, a recombinant REE-binding protein or EF-hand domain's activity may be determined by measuring its substrate specificity and/or ability to bind one or more REEs.

The skilled artisan will also realize that mutations in a recombinant peptide or polypeptide (e.g., EF-hand domain or REE-binding protein) coding sequence may result in conservative amino acid substitutions to provide functionally equivalent variants of such a peptide or polypeptide, i.e., variants that retain the same or similar activity. As used in this disclosure, a “conservative amino acid substitution” refers to an amino acid substitution that does not alter the relative charge or size characteristics or functional activity of the protein in which the amino acid substitution is made.

In some instances, an amino acid is characterized by its R group (see, e.g., Table 1). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group includes lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Variants can be prepared according to methods for altering polypeptide sequence known to one of ordinary skill in the art such as are found in references which compile such methods, e.g., Molecular Cloning: A Laboratory Manual, J. Sambrook, et al., eds., Fourth Edition, Cold Spring Harbor Laboratory Press, Cold Spring Harbor, N.Y., 2012, or Current Protocols in Molecular Biology, F. M. Ausubel, et al., eds., John Wiley & Sons, Inc., New York, 2010.

Non-limiting examples of functionally equivalent variants of polypeptides may include conservative amino acid substitutions in the amino acid sequences of proteins disclosed in this application. As used in this disclosure “conservative substitution” is used interchangeably with “conservative amino acid substitution” and refers to any one of the amino acid substitutions provided in Table 1.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions.

TABLE 1
Conservative Amino Acid Substitutions

| | | Conservative |
|---|---|---|
| Original | | Amino Acid |
| Residue | R Group Type | Substitutions |
| Ala | nonpolar aliphatic R group | Cys, Gly, Ser |
| Arg | positively charged R group | His, Lys |
| Asn | polar uncharged R group | Asp, Gln, Glu |
| Asp | negatively charged R group | Asn, Gln, Glu |
| Cys | polar uncharged R group | Ala, Ser |
| Gln | polar uncharged R group | Asn, Asp, Glu |
| Glu | negatively charged R group | Asn, Asp, Gin |
| Gly | nonpolar aliphatic R group | Ala, Ser |
| His | positively charged R group | Arg, Tyr, Trp |
| Ile | nonpolar aliphatic R group | Leu, Met, Val |
| Leu | nonpolar aliphatic R group | Ile, Met, Val |
| Lys | positively charged R group | Arg, His |
| Met | nonpolar aliphatic R group | Ile, Leu, Phe, Val |
| Pro | polar uncharged R group | |
| Phe | nonpolar aromatic R group | Met, Trp, Tyr |
| Ser | polar uncharged R group | Ala, Gly, Thr |
| Thr | polar uncharged R group | Ala, Asn, Ser |
| Trp | nonpolar aromatic R group | His, Phe, Tyr, Met |
| Tyr | nonpolar aromatic R group | His, Phe, Trp |
| Val | nonpolar aliphatic R group | Ile, Leu, Met, Thr |

Amino acid substitutions in the amino acid sequence of a peptide or polypeptide to produce a recombinant peptide or polypeptide (e.g., EF-hand domain or REE-binding protein) variant having a desired property and/or activity can be made by alteration of the coding sequence of the polypeptide (e.g., EF-hand domain or REE-binding protein). Similarly, conservative amino acid substitutions in the amino acid sequence of a polypeptide to produce functionally equivalent variants of the polypeptide typically are made by alteration of the coding sequence of the recombinant polypeptide (e.g., EF-hand domain or REE-binding protein).

Mutations (e.g., substitutions) can be made in a nucleotide sequence by any method known to one of ordinary skill in the art. For example, mutations can be made by PCR-directed mutation, site-directed mutagenesis according to the method of Kunkel (Kunkel, Proc. Nat. Acad. Sci. U.S.A. 82: 488-492, 1985), by chemical synthesis of a gene encoding a polypeptide, by gene editing techniques, or by insertions, such as insertion of a tag (e.g., a HIS tag or a GFP tag).

In some instances, the REE-binding protein is fused to an affinity tag, which may be useful in purification of the REE-binding protein. Non-limiting examples of affinity tags include albumin-binding protein (ABP), alkaline phosphatase (AP), chloramphenicol acetyl transferase (CAT), Glutathione S-transferase (GST), human influenza hemagglutinin (HA), maltose-binding protein (MBP), myc epitope, polyarginine (Arg-tag), polyhistidine (His-tag), biotin, streptavadin-binding peptide (SBP), streptavidin, and tandem Affinity Purification (TAP).

### Methods of Recovering REEs

Aspects of the present disclosure relate to the recombinant expression of genes encoding enzymes, functional modifications and variants thereof, as well as uses relating thereto. For example, the methods described in this disclosure may be used to recover an REE from a sample by contacting a sample with an REE-binding protein of the present disclosure. In some instances, host cells are used to secrete an REE-binding protein of interest and host cells are introduced into a sample of interest. In some embodiments, the REE-binding proteins are secreted from the host cells. The REE-binding protein may be further purified. In some embodiments, the REE-binding proteins of the present disclosure are used to bind an REE within a cell.

Samples may be of any source material. For example, samples include environmental samples from soil and waterways. In some embodiments, a sample is a stream sample, pond sample, soil sample, mine sample, river sample, mountain sample, or creek sample. In some embodiments, a sample is a synthetic sample comprising one or more metals. A sample can include any material in any location that comprises REEs. In some embodiments, a sample includes or is derived from machining waste. In some embodiments, a sample includes or is derived from electronic waste.

A nucleic acid encoding any of the recombinant polypeptides (e.g., REE-binding proteins) described in this disclosure may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a galactose-inducible vector (e.g., comprising a P_{gal} promoter) or doxycycline-inducible vector). In some instances, the vector is a lactose-inducible vector.

In some embodiments, a vector replicates autonomously in a cell. A vector can contain one or more endonuclease restriction sites that are cut by a restriction endonuclease to insert and ligate a nucleic acid containing a gene described in this disclosure to produce a recombinant vector that is able to replicate in a cell. Vectors are typically composed of DNA, although RNA vectors are also available. Cloning vectors include, but are not limited to: plasmids, fosmids, phagemids, virus genomes and artificial chromosomes. As used in this disclosure, the terms “expression vector” or “expression construct” refer to a nucleic acid construct, generated recombinantly or synthetically, with a series of specified nucleic acid elements that permit transcription of a particular nucleic acid in a host cell (e.g., microbe), such as a bacterial cell or a yeast cell. In some embodiments, the nucleic acid sequence of a gene described in this disclosure is inserted into a cloning vector such that it is operably joined to regulatory sequences and, in some embodiments, expressed as an RNA transcript. In some embodiments, the vector contains one or more markers, such as a selectable marker as described in this disclosure, to identify cells transformed or transfected with the recombinant vector. In some embodiments, the nucleic acid sequence of a gene described in this disclosure is codon-optimized. Codon-optimization of a sequence may increase production of a gene product by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all values in between relative to a reference sequence that is not codon-optimized.

A coding sequence and a regulatory sequence are said to be “operably joined” or “operably linked” when the coding sequence and the regulatory sequence are covalently linked and the expression or transcription of the coding sequence is under the influence or control of the regulatory sequence. If the coding sequence is to be translated into a functional protein, the coding sequence and the regulatory sequence are said to be operably joined if induction of a promoter in the 5′ regulatory sequence permits the coding sequence to be transcribed and if the nature of the linkage between the coding sequence and the regulatory sequence does not (1) result in the introduction of a frame-shift mutation, (2) interfere with the ability of the promoter region to direct the transcription of the coding sequence, or (3) interfere with the ability of the corresponding RNA transcript to be translated into a protein.

In some embodiments, the nucleic acid encoding any of the proteins described in this disclosure is under the control of regulatory sequences (e.g., enhancer sequences). In some embodiments, a nucleic acid is expressed under the control of a promoter. The promoter can be a native promoter, e.g., the promoter of the gene in its endogenous context, which provides normal regulation of expression of the gene. Alternatively, a promoter can be a promoter that is different from the native promoter of the gene, e.g., the promoter is different from the promoter of the gene in its endogenous context.

In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1, TPI1 GAL1, GAL10, GAL7, GAL3, GAL2, MET3, MET25, HXT3, HXT7, ACT1, ADH1, ADH2, CUP1-1, ENO2, and SOD1, as would be known to one of ordinary skill in the art (see, e.g., Addgene website: blog.addgene.org/plasmids-101-the-promoter-region).

In some embodiments, the promoter is a prokaryotic promoter (e.g., bacteriophage or bacterial promoter). Non-limiting examples of bacteriophage promoters include Pls1con, T3, T7, SP6, and PL. Non-limiting examples of bacterial promoters include apFAB101, apFAB92 (Ec-TTL-P100), abFAB71 (Ec-TTL-P097), apFAB45 (Ec-TTL-9092), apFAB29, apFAB76 (EC-TTL-P075), BBA_J23104 (Ec TTL-P054), J23104, Ec-TTL-P041, apFAB436 (Ec-TTL-P046), apFAB332, Pbad, PmgrB, Ptrc2, Plac/ara, Ptac, and Pm, as would be known to one of ordinary skill in the art.

In some embodiments, the promoter is an inducible promoter. As used in this disclosure, an “inducible promoter” is a promoter controlled by the presence or absence of a molecule. Non-limiting examples of inducible promoters include chemically regulated promoters and physically-regulated promoters. For chemically regulated promoters, the transcriptional activity can be regulated by one or more compounds, such as alcohol, tetracycline, galactose, a steroid, a metal, or other compounds. For physically regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination thereof.

In some embodiments, the promoter is a constitutive promoter. As used in this disclosure, a “constitutive promoter” refers to an unregulated promoter that allows continuous transcription of a gene. Non-limiting examples of a constitutive promoter include TDH3, PGK1, PKC1, PDC1, TEF1, TEF2, RPL18B, SSA1, TDH2, PYK1, TPI1, HXT3, HXT7, ACT1, ADH1, ADH2, ENO2, and SOD1.

Other inducible promoters or constitutive promoters known to one of ordinary skill in the art are also contemplated in this disclosure.

The precise nature of the regulatory sequences needed for gene expression may vary between species or cell types, but generally include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences. The vectors disclosed in this application may include 5′ leader or signal sequences. The regulatory sequence may also include a terminator sequence. In some embodiments, a terminator sequence marks the end of a gene in DNA during transcription. The choice and design of one or more appropriate vectors suitable for inducing expression of one or more genes described in this disclosure in a heterologous organism is within the ability and discretion of one of ordinary skill in the art.

An expression vector encoding one or more REE-binding proteins may further encode a secretion signal peptide that promotes the export of the REE-binding protein out of a cell. Depending on the cell type, a prokaryotic or eukaryotic signal peptide may be used. Secretion signal peptides are often located at the amino-terminal end of a polypeptide sequence. Non-limiting examples of secretion signal peptides in bacteria include general secretion (Sec) and twin arginine translocation (Tat) signal peptides that promote export through the general secretion (Sec) export pathway or through the Tat export pathway, respectively. See, e.g., Freudl Microb Cell Fact. 2018; 17: 52; Lee et al., Annu Rev Microbiol. 2006; 60: 373-395. Sec and Tat signal peptides comprise a tripartite structure and have a positively charged n-region, a central hydrophobic h-region, and a polar c-region that harbors the recognition site (consensus: A-X-A) for signal peptidase. Tat signal peptides comprise an amino acid consensus motif (S/T-R-R-X-F-L-K, where X often is a polar amino acid residue). As a non-limiting example, the secretion signal from S. cerevisiae pre-pro-α-factor, which is the precursor to a peptide mating pheromone, may be used to secrete a REE-binding protein from yeast. See also, e.g., Fitzgerald and Glick, Microb Cell Fact. 2014; 13: 125.

Expression vectors containing the necessary elements for expression are commercially available and known to one of ordinary skill in the art (see, e.g., Sambrook et al., Molecular Cloning: A Laboratory Manual, Fourth Edition, Cold Spring Harbor Laboratory Press, 2012).

### Host Cells

Any of the proteins of the present disclosure may be expressed in a host cell. As used in this disclosure, a host cell is a cell that can be used to express at least one heterologous polynucleotide (e.g., encoding a protein or enzyme as described in this application).

The term “heterologous” with respect to a polynucleotide, such as a polynucleotide comprising a gene, is used interchangeably with the term “exogenous” and the term “recombinant” and refers to: a polynucleotide that has been artificially supplied to a biological system; a polynucleotide that has been modified within a biological system, or a polynucleotide whose expression or regulation has been manipulated within a biological system. A heterologous polynucleotide that is introduced into or expressed in a host cell may be a polynucleotide that comes from a different organism or species than the host cell, or may be a synthetic polynucleotide, or may be a polynucleotide that is also endogenously expressed in the same organism or species as the host cell. For example, a polynucleotide that is endogenously expressed in a host cell may be considered heterologous when it is situated non-naturally in the host cell; expressed recombinantly in the host cell, either stably or transiently; modified within the host cell; selectively edited within the host cell; expressed in a copy number that differs from the naturally occurring copy number within the host cell; or expressed in a non-natural way within the host cell, such as by manipulating regulatory regions that control expression of the polynucleotide. In some embodiments, a heterologous polynucleotide is a polynucleotide that is endogenously expressed in a host cell but whose expression is driven by a promoter that does not naturally regulate expression of the polynucleotide. In other embodiments, a heterologous polynucleotide is a polynucleotide that is endogenously expressed in a host cell and whose expression is driven by a promoter that does naturally regulate expression of the polynucleotide, but the promoter or another regulatory region is modified. In some embodiments, the promoter is recombinantly activated or repressed. For example, gene-editing based techniques may be used to regulate expression of a polynucleotide, including an endogenous polynucleotide, from a promoter, including an endogenous promoter. See, e.g., Chavez et al., Nat Methods. 2016 July; 13(7): 563-567. A heterologous polynucleotide may comprise a wild-type sequence or a mutant sequence as compared with a reference polynucleotide sequence.

Any suitable host cell may be used to produce any of the recombinant polypeptides (REE-binding proteins) disclosed in this application, including eukaryotic cells or prokaryotic cells. Suitable host cells include bacteria cells (e.g., Escherichia coli cells or Bacillus cells) and fungal cells (e.g., yeast cells). Non-limiting examples of genera of bacteria cells include Yersinia spp., Escherichia spp., Klebsiella spp., Agrobacterium spp., Acinetobacter spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Lactococcus spp., Rhodococcus spp., Pseudomonas spp., Helicobacter spp., Salmonella spp., Vibrio spp., Bacillus spp., Erysipelothrix spp., Salmonella spp., Streptomyces spp., Bacteroides spp., Prevotella spp., Clostridium spp., Bifidobacterium spp., or Lactobacillus spp.

Non-limiting examples of genera of yeast for expression include Saccharomyces (e.g., S. cerevisiae), Pichia, Kluyveromyces (e.g., K. lactis), Hansenula and Yarrowia. In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

The term “cell,” as used in this disclosure, may refer to a single cell or a population of cells, such as a population of cells belonging to the same cell line or strain. Use of the singular term “cell” should not be construed to refer explicitly to a single cell rather than a population of cells.

The host cell may comprise genetic modifications relative to a wild-type counterpart. Reduction of gene expression and/or gene inactivation may be achieved through any suitable method, including but not limited to deletion of the gene, introduction of a point mutation into the endogenous gene, and/or truncation of the endogenous gene. For example, polymerase chain reaction (PCR)-based methods may be used (see, e.g., Gardner et al., Methods Mol Biol. 2014; 1205:45-78). As a non-limiting example, genes may be deleted through gene replacement (e.g., with a marker, including a selection marker). A gene may also be truncated through the use of a transposon system (see, e.g., Poussu et al., Nucleic Acids Res. 2005; 33(12): e104). Genes may also be modified through gene editing techniques known to one of ordinary skill in the art.

A vector encoding any of the recombinant polypeptides (e.g., REE-binding protein) described in this disclosure may be introduced into a suitable host cell using any method known in the art.

Non-limiting examples of bacteria transformation protocols are described in Hanahan Methods Enzymol. 1991; 204:63-113; Gerhardt, P. R., Murray, R. G. E., Wood, W. A. & Krieg, N. R. (editors) (1994). Methods for General and Molecular Bacteriology. Washington, D.C.: American Society for Microbiology; and Green, P. N. & Bousfield, I. J. (1982).

Non-limiting examples of yeast transformation protocols are described in Gietz et al., Yeast transformation can be conducted by the LiAc/SS Carrier DNA/PEG method. Methods Mol Biol. 2006; 313:107-20, which is hereby incorporated by reference in its entirety for this purpose.

Host cells may be cultured under any conditions suitable as would be understood by one of ordinary skill in the art. For example, any media, temperature, and incubation conditions known in the art may be used. For host cells carrying an inducible vector, cells may be cultured with an appropriate inducible agent to promote expression.

Any of the cells disclosed in this application can be cultured in media of any type (rich or minimal) and any composition prior to, during, and/or after contact and/or integration of a nucleic acid. The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the cells described in this disclosure can be performed in culture vessels known and used in the art. In some embodiments, an aerated reaction vessel (e.g., a stirred tank reactor) is used to culture the cells. In some embodiments, a bioreactor or fermenter is used to culture the cells. Thus, in some embodiments, the cells are used in fermentation. As used in this disclosure, the terms “bioreactor” and “fermenter” are interchangeably used and refer to an enclosure, or partial enclosure, in which a biological, biochemical and/or chemical reaction takes place, involving a living organism or part of a living organism. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

In some embodiments, a bioreactor comprises a cell (e.g., a bacteria cell or a yeast cell) or a cell culture (e.g., bacteria cell culture or yeast cell culture), such as a cell or cell culture described in this disclosure. In some embodiments, a bioreactor comprises a spore and/or a dormant cell type of an isolated microbe (e.g., a dormant cell in a dry state).

Non-limiting examples of bioreactors include: stirred tank fermenters, bioreactors agitated by rotating mixing devices, chemostats, bioreactors agitated by shaking devices, airlift fermenters, packed-bed reactors, fixed-bed reactors, fluidized bed bioreactors, bioreactors employing wave induced agitation, centrifugal bioreactors, roller bottles, and hollow fiber bioreactors, roller apparatuses (for example benchtop, cart-mounted, and/or automated varieties), vertically-stacked plates, spinner flasks, stirring or rocking flasks, shaken multi-well plates, MD bottles, T-flasks, Roux bottles, multiple-surface tissue culture propagators, modified fermenters, and coated beads (e.g., beads coated with serum proteins, nitrocellulose, or carboxymethyl cellulose to prevent cell attachment).

In some embodiments, the bioreactor includes a cell culture system where the cell (e.g., bacteria cell or yeast cell) is in contact with moving liquids and/or gas bubbles. In some embodiments, the cell or cell culture is grown in suspension. In other embodiments, the cell or cell culture is attached to a solid phase carrier. Non-limiting examples of a carrier system includes microcarriers (e.g., polymer spheres, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, encapsulation cells, capillaries, and aggregates. In some embodiments, carriers are fabricated from materials such as dextran, gelatin, glass, or cellulose.

In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion mode of operation. In some embodiments, a bioreactor allows continuous or semi-continuous replenishment of the substrate stock, for example a carbohydrate source and/or continuous or semi-continuous separation of the product, from the bioreactor.

In some embodiments, the bioreactor or fermenter includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO₂ concentration, nutrient concentrations, metabolite concentrations, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure the parameters described in this disclosure are well known to one of ordinary skill in the relevant mechanical and electronic arts. Control systems to adjust the parameters in a bioreactor based on the inputs from a sensor described in this disclosure are well known to one of ordinary skill in the art in bioreactor engineering.

In some embodiments, the method involves batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated. Also, the final product (e.g., an amino acid, including lysine) may display some differences from a naturally occurring product (e.g., an amino acid, including lysine) in terms of solubility, toxicity, chirality cellular accumulation and secretion and in some embodiments can have different fermentation kinetics.

In other embodiments, recovery of REEs may take place at a sample such as a mine or a waste stream, or in a processing center near a site such as a mine or waste stream. In some embodiments, REE-binding proteins, or cells containing REE-binding proteins, can be applied to a sample wherever the sample is located.

In some embodiments, the method comprises contacting a sample with a purified REE-binding protein to recover REEs. In some instances, host cells are engineered to produce recombinant REE-binding proteins, which may then been purified using methods known in the art. For example, REE-binding proteins comprising an affinity tag may be purified using affinity chromatography. Non-limiting examples of purification strategies include size exclusion chromatography, hydrophobic interaction chromatography, ion exchange chromatography, free-flow-electrophoresis, metal binding chromatography, immune-affinity chromatography, and high performance liquid chromatography.

In some embodiments, the methods further comprise purifying at least one REE that is bound to an REE-binding protein. In some instances, purifying an REE comprises extracting the REE. Non-limiting methods of REE purification include use of a metal chelator and treatment of an REE-bound REE-binding protein with denaturing conditions (e.g., altering the pH and/or temperature of the sample).

Further aspects of the disclosure relate to kits. Kits can include any REE-binding protein described in this disclosure and/or any protein comprising an EF-hand domain described in this disclosure. Kits may further comprise instructions for use of REE-binding proteins, such as use of an REE-binding protein for extraction of a rare earth element, such as lanthanide, scandium, or yttrium. The REE-binding protein for use in a kit may be an REE-binding protein that is expressed in, and secreted from, a cell. Kits may include cells for expressing REE-binding proteins and/or any materials necessary for producing cells or strains for expressing REE-binding proteins.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1. Identification of Candidate Lanthanide-Binding Proteins

To identify candidate rare-earth element (REE)-binding proteins, environmental samples comprising microbes were collected from REE mines. The samples were analyzed to identify genes encoding proteins comprising an EF-hand domain, which has previously been characterized to bind calcium. Samples were analyzed by sequencing, including both 16S (genotype) sequencing, as well as full metagenomic sequencing.

To assemble short 100-150 bp long reads into longer “contigs,” the Megahit tool (Li et al., Bioinformatics. 2015 May 15; 31(10):1674-6) was used to generate a so-called “co-assembly” of all the reads in a given dataset. The resulting contigs were filtered for length to remove short sequences (<1000 bp) that are unlikely to encode complete domains. Transdecoder (Haas et al., Nat Protoc. 2013 August; 8(8):1494-512) was used to predict potential open reading frames in the remaining dataset. These predicted sequences were then compared to the PFAM domains described in Table 3.

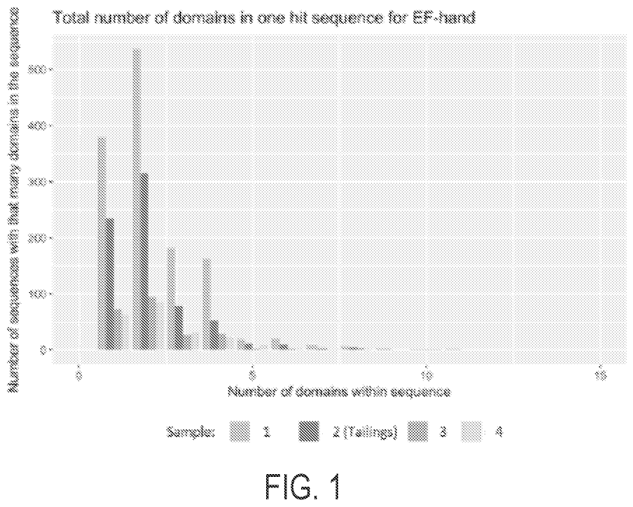

As shown in FIG. 1, multiple EF-hand domain containing proteins were identified. Most of these proteins contained approximately two EF-hand domains, while some of the proteins contained more than five EF-hand domains. This phenomenon was observed across all four datasets. The “Tailings” sample contained significant amounts of Dysprosium and other lanthanides.

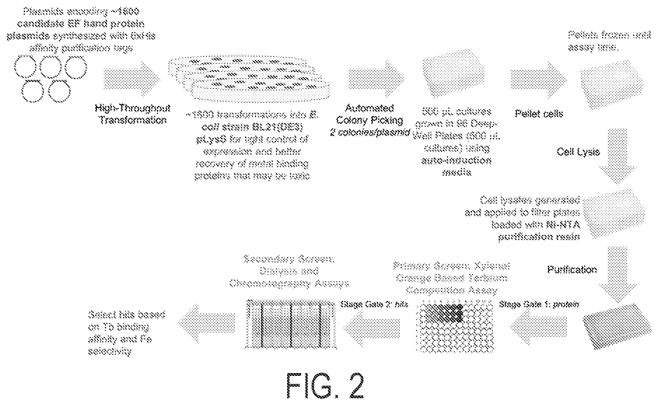

Candidate lanthanide-binding proteins comprising one or more EF-hand domains, identified from the sequence studies, were further characterized. An overview of the methods used to characterize the candidate lanthanide-binding proteins is provided in FIG. 2. As shown in FIG. 2, library proteins were expressed in E. coli BL21(DE3) cells, which express T7 RNA polymerase upon induction with lactose metabolites. Approximately 1600 EF-hand proteins were encoded into plasmids and placed under control of the T7 promoter, thereby allowing protein production to be controlled by media conditions. To alleviate potential concerns regarding cellular toxicity of metal-binding proteins, BL21(DE3) pLysS was selected for the primary screen. E. coli BL21(DE3) cells were transformed with plasmids containing EF-hand domain containing proteins. The resulting strains were grown in 96 deep-well plates in autoinduction media selected for maximal protein expression, then subsequently pelleted and stored frozen.

The EF-hand domain-containing proteins were purified with immobilized metal-ion affinity chromatography using a high-throughput 96-well plate-based purification protocol. The EF-hand domain containing proteins contained a polyhistidine ‘tag’ sequence, allowing the proteins to be purified by lysing the cells and separating the proteins through the selective and high-affinity binding of polyhistidine to Ni-NTA resin. Following separation from cellular debris, the purified proteins were removed from resin by eluting in imidazole buffer. Purified candidate lanthanide-binding proteins were used in all experimental assays.

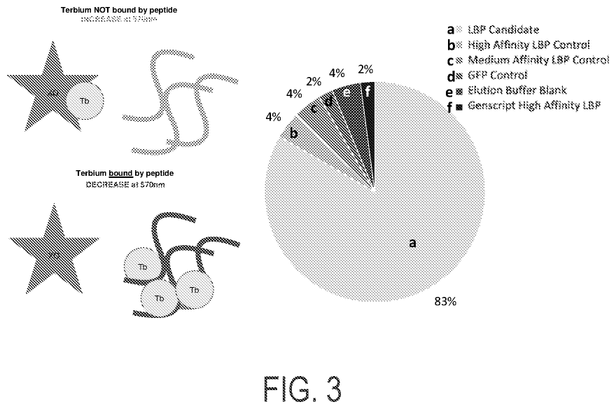

In the primary screen, the entire protein library was screened for terbium (Tb)-binding using a competition assay between candidate proteins and the metal-binding colorimetric indicator Xylenol Orange (XO). FIG. 3 depicts the competition assay featuring the chromophore XO used in the primary screen. As shown in the left panel of FIG. 3, the chromophore XO exhibits an increase in absorption at 570 nm (Abs570) when complexed with Tb. In situations with weak Tb-binding proteins, the majority of Tb is bound to XO, leading to an increase in Abs570 (FIG. 3, top left). Strong Tb-binding proteins sequester the metal away from XO, which decreases Abs570 (FIG. 3, bottom left). The Tb affinity of 1461 candidate lanthanide-binding proteins was measured. The distribution of controls and lanthanide-binding proteins (LBPs) is shown in the pie chart in FIG. 3.

There were two biological replicates for a unique protein, and each protein was measured in triplicate. The screen contained three positive controls, one negative control, and buffer only sample blank. These controls were included on each of the 96-well assay plates to ensure experimental consistency and to normalize for any systematic differences between plates. Twenty percent of samples in the screen were controls. In total, the primary screen comprised over 10,809 samples on 114 96-well plates.

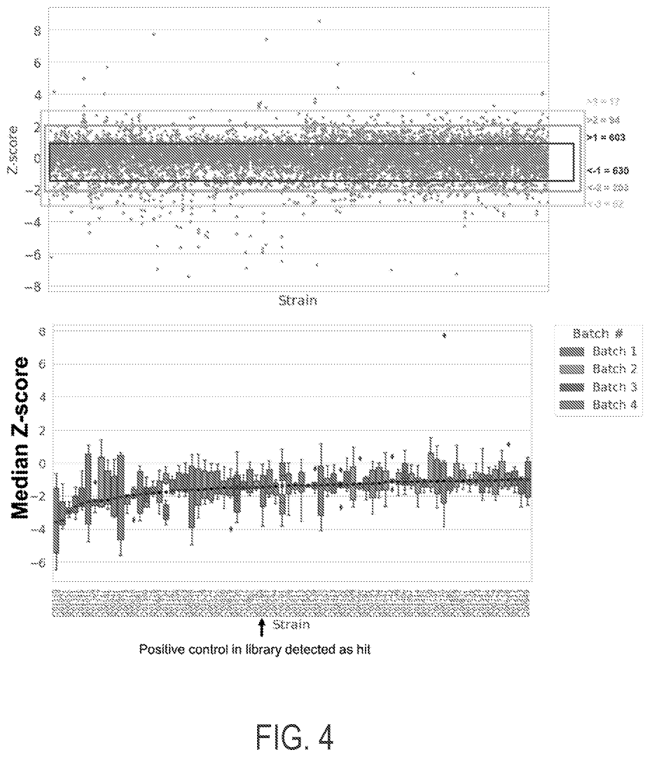

Data from the primary screen are shown in FIG. 4. To account for systematic differences between sample batches and plates, the Abs570 measurements were transformed to Z-scores by normalizing each value to the plate mean and standard deviation. Proteins with strong Tb-binding affinity are expected to sequester Tb ions away from XO, which will lead to a decrease in Abs570 and correspondingly negative Z-score. Indeed, there was a relative enrichment of samples with Z-scores 2 and 3 standard deviations below the mean, consistent with Tb-binding by candidate proteins (referred to sometimes as “strains”). These data confirmed that the primary screen identified Tb-binding proteins from experimental trials conducted on different days (corresponding to different batches). Overall, several hundred proteins displayed average Z-scores less than −1 (FIG. 4, bottom).

The primary screen hits—Tb-binding proteins—were selected for a secondary screen, which used a dialysis assay to directly measure Tb and dysprosium binding affinity and the selective binding of lanthanide metals over iron.

### Example 2. Characterization of Candidate Lanthanide-Binding Proteins

Out of the Tb-binding proteins identified in Example 1, 294 proteins were chosen for the secondary library, based on the criteria that selected strains must have 2+ replicates and Z-scores less than 1 standard deviation below the mean.

In developing a more sensitive secondary screen, an experimental approach was developed to measure metal binding directly. The secondary library was screened for Tb-binding using a dialysis-plate assay. For the dialysis assay, protein solutions were dialyzed overnight in 50× excess volume containing 2 μM Tb. After removing the protein solutions from the dialysis chamber their concentrations were measured by absorption at 280 nm (Abs280) then treated with Delfia reagents to sequester and concentrate Tb for quantitation by Delfia fluorescence assay. Fluorescence intensities were collected using a plate reader by measuring emission at 545 nm after excitation at 320 nm. Inset shows the 96-well dialysis plate setup.

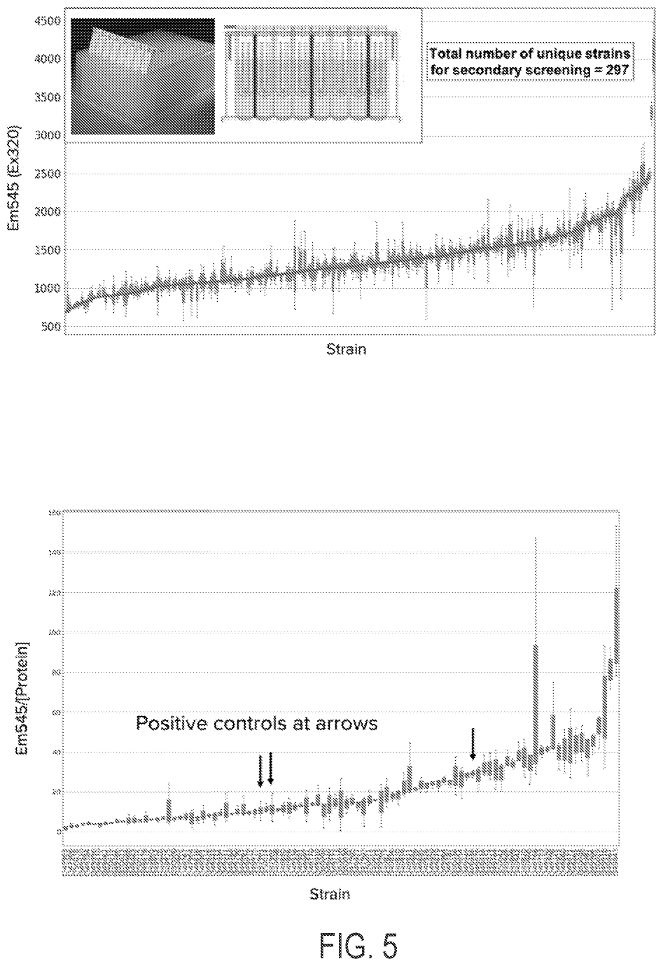

In total, the screen contained 297 proteins, including the 294 candidate lanthanide-binding protein plus three positive controls. Sample buffer-only blanks were also screened (FIG. 5, left graph). Each protein was measured in triplicate. The measured intensity values are directly proportional to the Tb concentration in the protein solution, which will increase if a protein binds Tb. The values vary from slightly above background (FIG. 5, left-hand side of left graph) to substantially increased (right-hand side of left graph), indicating that many of the secondary screen library proteins bound Tb. To account for differences in protein expression levels between strains, the Tb binding data were normalized by protein concentration (FIG. 5, right graph), and proteins were selected with concentrations above 20 μM to focus on ones that express well and to minimize artifacts associated with normalization. See also Table 3. Many of the secondary library proteins had Tb affinities comparable to or better than the three positive controls, which included two engineered lanthanide-binding proteins and a natural protein with reported picomolar lanthanide binding affinity. These results confirmed the hits selected from the primary screen and provided a direct experimental metric to rank Tb-binding by library proteins.

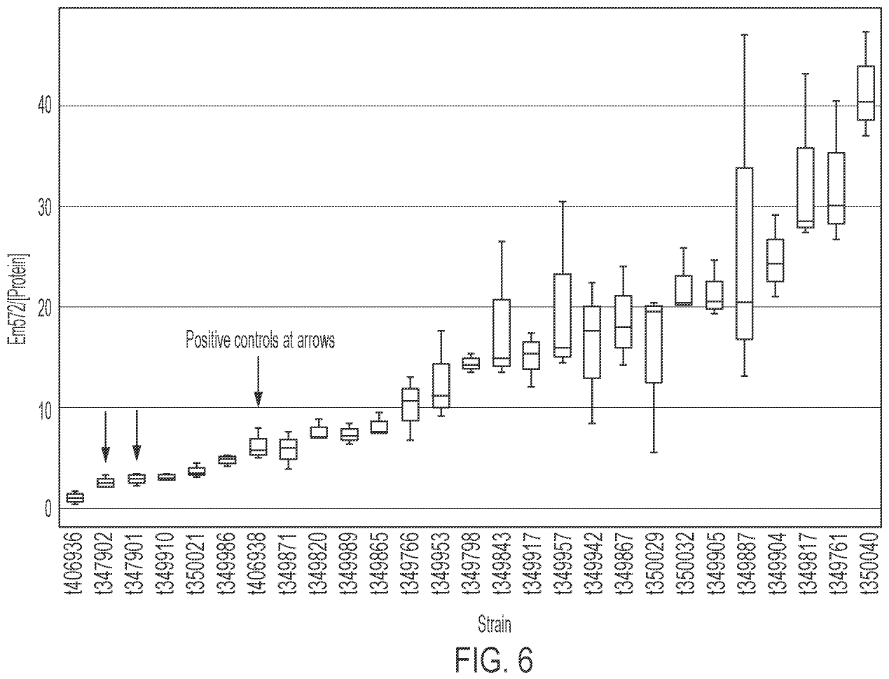

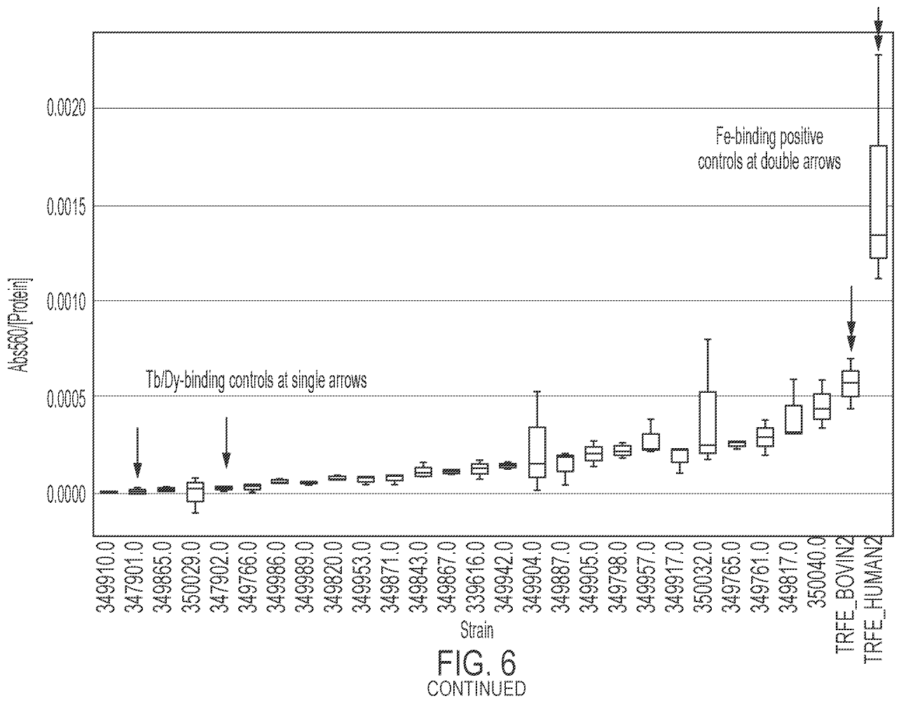

To determine whether the discovered Tb-binding proteins also bind other lanthanides, identical dialysis-plate assays were conducted using dysprosium on the top 23 ‘best-binder’ proteins (FIG. 6, left). Consistent with their performance in the Tb experiments, the best binders also demonstrated high affinity for dysprosium; the majority of them outperformed the positive controls. Dialysis-plate assays using the transition metal iron were also performed on the best binders to assess their metal binding selectivity (FIG. 6, right). See also Table 3. In Table 3, the absence of a value indicates that the property was not measured.

It was also investigated whether the Tb-binding proteins also bind to iron. Iron was detected using a Ferrozine chromogenic assay kit commonly used to measure iron bound by proteins in biological samples. This assay involves denaturing the proteins to release bound iron, which can then form a strongly colorimetric complex with ferrozine. The assay can detect micromolar (or lower) metal concentrations. Most of the best-binder proteins did not bind iron at micromolar concentrations and, therefore, displayed selectivity for lanthanides over iron.

The primary and secondary screens revealed that proteins comprising a sequence selected from SEQ ID NOs: 1-179 are lanthanide-binding proteins. The secondary screen showed that proteins comprising a sequence selected from SEQ ID NOS: 4-71 have binding affinities that are comparable to or greater than control lanthanide binding proteins (SEQ ID NOs: 2-3). Binding levels of up to five times better than controls was observed.

The additional sensitivity of the secondary screen confirmed the lanthanide binding abilities of proteins identified in the primary screen and allowed for the comparison of lanthanide affinities to previously reported lanthanide binding proteins. In total, over 20 proteins that bind terbium and dysprosium metals better than previously described natural and designed lanthanide-binding proteins were identified. Moreover, most of the high affinity lanthanide-binding proteins identified in this disclosure did not bind iron at micromolar concentrations, indicating selectivity for lanthanides over iron.

### Sequences of Candidate Lanthanide-Binding Proteins

Table 2 provides the sequences of proteins described in Examples 1 and 2. In Table 2, the lanthanide (Ln)-binding affinity of each amino acid sequence is indicated. “Control” indicates a control Ln-binding protein (t347901 and/or t347902), “Best” indicates binding affinity comparable to or greater than strains t347901 and/or t347902, “hit” indicates binding affinity signal significantly above background, but less than t347901 and t347902, and “weak” indicates metal-binding signal similar to background level as detected in the assays. The initial primary screen was conducted on a library of 1,461 proteins and used t347901 and t347902 as control Ln-binding proteins. The top performing proteins from the primary screen (297 proteins) were advanced to the secondary screen. In addition to the t347901 and t347902 controls, t406938 was also included as a control protein in the secondary screen. The “best, “hit,” and “weak” designations were made based on the Terbium-binding assay conducted in the secondary screen. All proteins that advanced to the secondary screen tested positive for Tb-binding in the primary screen.

TABLE 2
Sequences

| | | | | | Number | |
|---|---|---|---|---|---|---|
| | Amino | | | | of EF- | Ln- |
| | Acid | Nucleotide | Position of EF- | EF-hand domain | hand | binding |
| Strain | Sequence | Sequence | hand domains | sequences | domains | Affinity |
| t406938 | (SEQ ID | (SEQ ID | (49, 61)(25, 37) | DPDKDGTLDAKEL | 4 | Control |
| | NO: 1) | NO: 978) | (98, 110)(74, 86) | (SEQ ID NO: 300) | | |
| | | | | DPDKDGTIDLKEA | | |
| | | | | (SEQ ID NO: 301) | | |
| | | | | NPDNDGTIDAREL | | |
| | | | | (SEQ ID NO: 302) | | |
| | | | | DPDNDGTLDKKEY | | |
| | | | | (SEQ ID NO: 303) | | |
| t347901 | (SEQ ID | (SEQ ID | | | 0 | Control |
| | NO: 2) | NO: 979) | | | | |
| t347902 | (SEQ ID | (SEQ ID | | | 0 | Control |
| | NO: 3) | NO: 980) | | | | |
| t349871 | (SEQ ID | (SEQ ID | (65,77)(91, 103) | DGNGDNMVSLQEF | 2 | Best |
| | NO: 4) | NO: 981) | | (SEQ ID NO: 304) | | |
| | | | | DANRDRRLSDDEY | | |
| | | | | (SEQ ID NO: 305) | | |
| t349865 | (SEQ ID | (SEQ ID | (47, 59)(173, | DTQGQGYLEKSDL | 3 | Best |
| | NO: 5) | NO: 982) | 185)(82, 94) | (SEQ ID NO: 306) | | |
| | | | | DTDGDGKVSFTEA | | |
| | | | | (SEQ ID NO: 307) | | |
| | | | | DADSDGKLTQTEL | | |
| | | | | (SEQ ID NO: 308) | | |
| t349953 | (SEQ ID | (SEQ ID | (122, 134)(66, | DADGDGVVTREEF | 3 | Best |
| | NO: 6) | NO: 983) | 78)(98, 110) | (SEQ ID NO: 309) | | |
| | | | | DADGDGYISFEEL | | |
| | | | | (SEQ ID NO: 310) | | |
| | | | | DSDGDGRVSVAEF | | |
| | | | | (SEQ ID NO: 311) | | |
| t349867 | (SEQ ID | (SEQ ID | (86, 98)(176, | DADSDGKLTQDEM | 3 | Best |
| | NO: 7) | NO: 984) | 188)(49,61) | (SEQ ID NO: 312) | | |
| | | | | DANGDGRVTASEA | | |
| | | | | (SEQ ID NO: 313) | | |
| | | | | DTSSKGYLEQADF | | |
| | | | | (SEQ ID NO: 314) | | |
| t350021 | (SEQ ID | (SEQ ID | (91, 103)(111, | DANGDGVITPDEA | 4 | Best |
| | NO: 8) | NO: 985) | 123)(135, 147) | (SEQ ID NO: 315) | | |
| | | | (67, 79) | DANGDGQITADEL | | |
| | | | | (SEQ ID NO: 316) | | |
| | | | | DANGDGVVTKEER | | |
| | | | | (SEQ ID NO: 317) | | |
| | | | | DTDKDGNISAEEA | | |
| | | | | (SEQ ID NO: 318) | | |
| t349910 | (SEQ ID | (SEQ ID | (66, 78)(43, 55) | DNNGDGIVNLGEA | 2 | Best |
| | NO: 9) | NO: 986) | | (SEQ ID NO: 319) | | |
| | | | | DSNADGVLSIEEA | | |
| | | | | (SEQ ID NO: 320) | | |
| t349766 | (SEQ ID | (SEQ ID | (13,25)(90, 102) | DKDSDGIISLEEF | 4 | Best |
| | NO: 10) | NO: 987) | (126, 138)(49, | (SEQ ID NO: 321) | | |
| | | | 61) | DKDGNGFISKEEL | | |
| | | | | (SEQ ID NO: 322) | | |
| | | | | DKDGNGEIDYEEF | | |
| | | | | (SEQ ID NO: 323) | | |
| | | | | DTDANGVIDFGEF | | |
| | | | | (SEQ ID NO: 324) | | |
| t349817 | (SEQ ID | (SEQ ID | (68, 80)(44, 56) | DADGDGAISSEEH | 2 | Best |
| | NO: 11) | NO: 988) | | (SEQ ID NO: 325) | | |
| | | | | DRNRDGKITRDEV | | |
| | | | | (SEQ ID NO: 326) | | |
| t349905 | (SEQ ID | (SEQ ID | (192, 204)(127, | DRDGDGVLSRQER | 6 | Best |
| | NO: 12) | 989) | 139)(157, 169) | (SEQ ID NO: 327) | | |
| | | | (227, 239)(260, | DTNGDGVVDEDER | | |
| | | | 272)(287, 299) | (SEQ ID NO: 328) | | |
| | | | | DSNGDGILDEAER | | |
| | | | | (SEQ ID NO: 329) | | |
| | | | | DQDGDGELNADEQ | | |
| | | | | (SEQ ID NO: 330) | | |
| | | | | SNGDGRINTTDL | | |
| | | | | (SEQ ID NO: 331) | | |
| | | | | DVNNDGVVDALDV | | |
| | | | | (SEQ ID NO: 332) | | |
| t350032 | (SEQ ID | (SEQ ID | (96, 108)(72, 84) | DADDDGKLVLNEF | 6 | Best |
| | NO: 13) | NO: 990) | (156, 168)(120, | (SEQ ID NO: 333) | | |
| | | | 132)(180, 192) | DTDGNAVLTVTEH | | |
| | | | (204, 216) | (SEQ ID NO: 334) | | |
| | | | | DANGDGIIDDDEC | | |
| | | | | (SEQ ID NO: 335) | | |
| | | | | DKDGDGALIAKEY | | |
| | | | | (SEQ ID NO: 336) | | |
| | | | | QNADGKITLDEF | | |
| | | | | (SEQ ID NO: 337) | | |
| | | | | DQNRDGFISIEEH | | |
| | | | | (SEQ ID NO: 338) | | |
| t349904 | (SEQ ID | (SEQ ID | (120, 132)(96, | DKDGDGALIAKEY | 6 | Best |
| | NO: 14) | NO: 991) | 108)(156, 168) | (SEQ ID NO: 339) | | |
| | | | (204, 216)(72, | DADDDGKLVLNEF | | |
| | | | 84)(180, 192) | (SEQ ID NO: 340) | | |
| | | | | DANGDGIIDDDEC | | |
| | | | | (SEQ ID NO: 341) | | |
| | | | | DQNRDGFISIEEH | | |
| | | | | (SEQ ID NO: 342) | | |
| | | | | TDGNAVLTVTEH | | |
| | | | | (SEQ ID NO: 343) | | |
| | | | | DQNADGKITLDEF | | |
| | | | | (SEQ ID NO: 344) | | |
| t349917 | (SEQ ID | (SEQ ID | (173, 185)(200, | DIDGDGQLTLFDF | 2 | Best |
| | NO: 15) | NO: 992) | 212) | (SEQ ID NO: 345) | | |
| | | | | DFDGDGSLTLFDF | | |
| | | | | (SEQ ID NO: 346) | | |
| t349989 | (SEQ ID | (SEQ ID | (31,43)(68, 80) | DADGNGTIDFPEF | 3 | Best |
| | NO: 16) | NO: 993) | (104, 116) | (SEQ ID NO: 347) | | |
| | | | | DKDGNGYISAAEL | | |
| | | | | (SEQ ID NO: 348) | | |
| | | | | DIDGDGQVNYEGK | | |
| | | | | (SEQ ID NO: 349) | | |
| t349986 | (SEQ ID | (SEQ ID | (72, 84)(97, 109) | DRNGDGKLSRDEL | 2 | Best |
| | NO: 17) | NO: 994) | | (SEQ ID NO: 350) | | |
| | | | | DTDDDGLLTREEV | | |
| | | | | (SEQ ID NO: 351) | | |
| t350040 | (SEQ ID | (SEQ ID | (488, 500)(168, | DFDGDGQLTLFDF | 5 | Best |
| | NO: 18) | NO: 995) | 180)(70, 82) | (SEQ ID NO: 352) | | |
| | | | (265, 277)(119, | DLDGDGDIDLALT | | |
| | | | 131) | (SEQ ID NO: 353) | | |
| | | | | DFDGNGIIDLAIV | | |
| | | | | (SEQ ID NO: 354) | | |
| | | | | DVNGDGHPDIITA | | |
| | | | | (SEQ ID NO: 355) | | |
| | | | | DLNGDGHADLVTA | | |
| | | | | (SEQ ID NO: 356) | | |
| t349942 | (SEQ ID | (SEQ ID | (358, 370)(103, | DRDNDGRIRVKDI | 3 | Best |
| | NO: 19) | NO: 996) | 115)(321,333) | (SEQ ID NO: 357) | | |
| | | | | DQEGVENLTYEEL | | |
| | | | | (SEQ ID NO: 358) | | |
| | | | | SKSNDEVITLEDL | | |
| | | | | (SEQ ID NO: 359) | | |
| t349798 | (SEQ ID | (SEQ ID | (132, 144) | KVDRNGFIDLEEL | 1 | Best |
| | NO: 20) | NO: 997) | | (SEQ ID NO: 360) | | |
| t349843 | (SEQ ID | (SEQ ID | (91, 103)(126, | DANADGVLDDAEL | 2 | Best |
| | NO: 21) | NO: 998) | 138) | (SEQ ID NO: 361) | | |
| | | | | DKNKDGKIDKQEA | | |
| | | | | (SEQ ID NO: 362) | | |
| t349820 | (SEQ ID | (SEQ ID | (42, 54)(66, 78) | DADQDGVVTVTEF | 2 | Best |
| | NO: 22) | NO: 999) | | (SEQ ID NO: 363) | | |
| | | | | DINEDGVVDREEM | | |
| | | | | (SEQ ID NO: 364) | | |
| t349761 | (SEQ ID | (SEQ ID | (80, 92) | DKDKDRVIDPEEI | 1 | Best |
| | NO: 23) | NO: 1000) | | (SEQ ID NO: 365) | | |
| t349957 | (SEQ ID | (SEQ ID | (362, 374)(338, | DTGKTRMLGQNEF | 4 | Best |
| | NO: 24) | NO: 1001) | 350)(296, 308) | (SEQ ID NO: 366) | | |
| | | | (272, 284) | DTDHDGKVSREEF | | |
| | | | | (SEQ ID NO: 367) | | |
| | | | | DADASGALDNKEF | | |
| | | | | (SEQ ID NO: 368) | | |
| | | | | DTNKDGMVSKAEW | | |
| | | | | (SEQ ID NO: 369) | | |
| t349887 | (SEQ ID | (SEQ ID | (376, 388)(339, | DTDKDGKILISDI | 2 | Best |
| | NO: 25) | NO: 1002) | 351) | (SEQ ID NO: 370) | | |
| | | | | CPDRDGSLTVDQL | | |
| | | | | (SEQ ID NO: 371) | | |
| t350029 | (SEQ ID | (SEQ ID | (84, 96)(164, | DSDQDGYHYRGGM | 3 | Best |
| | NO: 26) | NO: 1003) | 176)(140, 152) | (SEQ ID NO: 372) | | |
| | | | | DANKDGRLTPEEM | | |
| | | | | (SEQ ID NO: 373) | | |
| | | | | DADGDGKLSLQEF | | |
| | | | | (SEQ ID NO: 374) | | |
| t347905 | (SEQ ID | (SEQ ID | | | 0 | Best |
| | NO: 27) | NO: 1004) | | | | |
| t349915 | (SEQ ID | (SEQ ID | (532, 544)(504, | DLNSNGAVTRVEL | 2 | Best |
| | NO: 28) | NO: 1005) | 516) | (SEQ ID NO: 375) | | |
| | | | | DSDGSGALSIAEA | | |
| | | | | (SEQ ID NO: 376) | | |
| t349893 | (SEQ ID | (SEQ ID | (723, 735)(755, | DANNDGTVDFADL | 2 | Best |
| | NO: 29) | NO: 1006) | 767) | (SEQ ID NO: 377) | | |
| | | | | DVNNDGVVDFADL | | |
| | | | | (SEQ ID NO: 378) | | |
| t349889 | (SEQ ID | (SEQ ID | (807,819)(775, | DVNGDGVVNFTDL | 2 | Best |
| | NO: 30) | NO: 1007) | 787) | (SEQ ID NO: 379) | | |
| | | | | DLDGDGEIGFGDL | | |
| | | | | (SEQ ID NO: 380) | | |
| t349961 | (SEQ ID | (SEQ ID | (253, 265)(280, | DIDGDGSLTLFDF | 2 | Best |
| | NO: 31) | NO: 1008) | 292) | (SEQ ID NO: 381) | | |
| | | | | DFDGDGSLTLFDF | | |
| | | | | (SEQ ID NO: 382) | | |
| t349916 | (SEQ ID | (SEQ ID | (437, 449)(410, | DQNQNGVVDLIDF | 2 | Best |
| | NO: 32) | NO: 1009) | 422) | (SEQ ID NO: 383) | | |
| | | | | DLNGDKRVNLTDF | | |
| | | | | (SEQ ID NO: 384) | | |
| t349765 | (SEQ ID | (SEQ ID | (88, 100) | DLNKDGMLSTEEF | 1 | Best |
| | NO: 33) | NO: 1010) | | (SEQ ID NO: 385) | | |
| t349909 | (SEQ ID | (SEQ ID | (409, 421)(382, | DQNQNGTVDLIDF | 2 | Best |
| | NO: 34) | NO: 1011) | 394) | (SEQ ID NO: 386) | | |
| | | | | DLNKDGSVNLTDF | | |
| | | | | (SEQ ID NO: 387) | | |
| t349980 | (SEQ ID | (SEQ ID | (73, 85) | DENGDGELSREEL | 1 | Best |
| | NO: 35) | NO: 1012) | | (SEQ ID NO: 388) | | |
| t349928 | (SEQ ID | (SEQ ID | (691,703)(663, | DLNNDGVVNFADL | 3 | Best |
| | NO: 36) | NO: 1013) | 675)(370, 382) | (SEQ ID NO: 389) | | |
| | | | | DTNGDSQVNFTDL | | |
| | | | | (SEQ ID NO: 390) | | |
| | | | | DVNGDTNVNLQDL | | |
| | | | | (SEQ ID NO: 391) | | |
| t349764 | (SEQ ID | (SEQ ID | (134, 146) | DKNKDGKISGADF | 1 | Best |
| | NO: 37) | NO: 1014) | | (SEQ ID NO: 392) | | |
| t349937 | (SEQ ID | (SEQ ID | (73, 85)(97, 109) | DADKDGVITLEEF | 7 | Best |
| | NO: 38) | NO: 1015) | (129, 141)(153, | (SEQ ID NO: 393) | | |
| | | | 165)(224, 236) | DKNGDGAIDRAEF | | |
| | | | (256, 268)(280, | (SEQ ID NO: 394) | | |
| | | | 292) | DADGDGRIGKEEF | | |
| | | | | (SEQ ID NO: 395) | | |
| | | | | DVDGDGQIGLEDM | | |
| | | | | (SEQ ID NO: 396) | | |
| | | | | KNGDGFIDAKDL | | |
| | | | | (SEQ ID NO: 397) | | |
| | | | | DADGDGKVTKDEF | | |
| | | | | (SEQ ID NO: 398) | | |
| | | | | DIDGDGKITDADL | | |
| | | | | (SEQ ID NO: 399) | | |
| t349936 | (SEQ ID | (SEQ ID | (133, 145)(160, | DSDHDGKVSRDEF | 2 | Best |
| | NO: 39) | NO: 1016) | 172) | (SEQ ID NO: 400) | | |
| | | | | DADHDGFISEAEA | | |
| | | | | (SEQ ID NO: 401) | | |
| t49954 | (SEQ ID | (SEQ ID | (53, 65)(85, 97) | DKNADGKVCLDEF | 2 | Best |
| | NO: 40) | NO: 1017) | | (SEQ ID NO: 402) | | |
| | | | | DKDADGFLTLEEF | | |
| | | | | (SEQ ID NO: 403) | | |
| t349797 | (SEQ ID | (SEQ ID | (693,705)(721, | DTNGDNVVNFTDL | 2 | Best |
| | NO: 41) | NO: 1018) | 733) | (SEQ ID NO: 404) | | |
| | | | | DIDGSGTVDFTDL | | |
| | | | | (SEQ ID NO: 405) | | |
| t349931 | (SEQ ID | (SEQ ID | (531,543) | DTNNNGELSEAEL | 1 | Best |
| | NO: 42) | NO: 1019) | | (SEQ ID NO: 406) | | |
| t349866 | (SEQ ID | (SEQ ID | (240, 252)(203, | DSNGDGEIDASDR | 2 | Best |
| | NO: 43) | NO: 1020) | 215) | (SEQ ID NO: 407) | | |
| | | | | DLNQNGRVDGGRE | | |
| | | | | (SEQ ID NO: 408) | | |
| t350037 | (SEQ ID | (SEQ ID | (159, 171)(128, | DLDGNEQVDFYDL | 2 | Best |
| | NO: 44) | NO: 1021) | 140) | (SEQ ID NO: 409) | | |
| | | | | DIDGDGLVGFTDL | | |
| | | | | (SEQ ID NO: 410) | | |
| t349819 | (SEQ ID | (SEQ ID | (534, 546)(561, | DLDGDGVLTLFDF | 2 | Best |
| | NO: 45) | NO: 1022) | 573) | (SEQ ID NO: 411) | | |
| | | | | DFDGDGALTLFDF | | |
| | | | | (SEQ ID NO: 412) | | |
| t350030 | (SEQ ID | (SEQ ID | (65, 77)(37, 49) | DVNKDGVVDAADA | 2 | Best |
| | NO: 46) | NO: 1023) | | (SEQ ID NO: 413) | | |
| | | | | DINKDGKVDLEDA | | |
| | | | | (SEQ ID NO: 414) | | |
| t349933 | (SEQ ID | (SEQ ID | (262, 274) | DSNGDGQIDSNAL | 1 | Best |
| | NO: 47) | NO: 1024) | | (SEQ ID NO: 415) | | |
| t349939 | (SEQ ID | (SEQ ID | (499, 511)(533, | DKDNDGRVTRDEW | 2 | Best |
| | NO: 48) | NO: 1025) | 545) | (SEQ ID NO: 416) | | |
| | | | | DKNNDGAWTLDEI | | |
| | | | | (SEQ ID NO: 417) | | |
| t350035 | (SEQ ID | (SEQ ID | (370, 382)(402, | DLDYSGTVNFEDL | 2 | Best |
| | NO: 49) | NO: 1026) | 414) | (SEQ ID NO: 418) | | |
| | | | | DINKDGFVNFTDM | | |
| | | | | (SEQ ID NO: 419) | | |
| t349818 | (SEQ ID | (SEQ ID | (76, 88)(53, 65) | DGNQDGVISKADV | 2 | Best |
| | NO: 50) | NO: 1027) | | (SEQ ID NO: 420) | | |
| | | | | DANADGVLNASEA | | |
| | | | | (SEQ ID NO: 421) | | |
| t349835 | (SEQ ID | (SEQ ID | (126, 138) | DHNGDGKVSWNEY | 1 | Best |
| | NO: 51) | NO: 1028) | | (SEQ ID NO: 422) | | |
| t349981 | (SEQ ID | (SEQ ID | (80, 92)(56, 68) | DTSGDGNLDRAEL | 2 | Best |
| | NO: 52) | NO: 1029) | | (SEQ ID NO: 423) | | |
| | | | | DTDGNGMLSREEL | | |
| | | | | (SEQ ID NO: 424) | | |
| t349846 | (SEQ ID | (SEQ ID | (86, 98)(134, | DRNRDLKITRREM | 3 | Best |
| | NO: 53) | NO: 1030) | 146)(110, 122) | (SEQ ID NO: 425) | | |
| | | | | DLNRDQELTQAEF | | |
| | | | | (SEQ ID NO: 426) | | |
| | | | | DKDGNNLLTFEEW | | |
| | | | | (SEQ ID NO: 427) | | |
| t349944 | (SEQ ID | (SEQ ID | (221,233) | DLDGDGTIDLKEW | 1 | Best |
| | NO: 54) | NO: 1031) | | (SEQ ID NO: 428) | | |
| t349874 | (SEQ ID | (SEQ ID | (157, 169)(287, | DSNGDGILDEAER | 6 | Best |
| | NO: 55) | NO: 1032) | 299)(260, 272) | (SEQ ID NO: 429) | | |
| | | | (127, 139)(192, | DVNNDGVVDALDV | | |
| | | | 204)(227, 239) | (SEQ ID NO: 430) | | |
| | | | | DSNGDGRINTTDL | | |
| | | | | (SEQ ID NO: 431) | | |
| | | | | DTNGDGVVDEDER | | |
| | | | | (SEQ ID NO: 432) | | |
| | | | | RDGDGVLSRQER | | |
| | | | | (SEQ ID NO: 433) | | |
| | | | | DQDGDGELNADEQ | | |
| | | | | (SEQ ID NO: 434) | | |
| t350022 | (SEQ ID | (SEQ ID | (159, 171)(135, | DRNDDGAIDLEDA | 4 | Best |
| | NO: 56) | NO: 1033) | 147)(103, 115) | (SEQ ID NO: 435) | | |
| | | | (79,91) | DVDGDGQVTIDEF | | |
| | | | | (SEQ ID NO: 436) | | |
| | | | | DADGDGQLSLAEY | | |
| | | | | (SEQ ID NO: 437) | | |
| | | | | DADGDGKVTQAEV | | |
| | | | | (SEQ ID NO: 438) | | |
| t349979 | (SEQ ID | (SEQ ID | (109, 121)(83, | DRSRDGKLDREEF | 2 | Best |
| | NO: 57) | NO: 1034) | 95) | (SEQ ID NO: 439) | | |
| | | | | DRNKDGFVDKSES | | |
| | | | | (SEQ ID NO: 440) | | |
| t350009 | (SEQ ID | (SEQ ID | (553, 565) | DSDGDGHISQAEW | 1 | Best |
| | NO: 58) | NO: 1035) | | (SEQ ID NO: 441) | | |
| t349785 | (SEQ ID | (SEQ ID | (209, 221)(115, | DTNGDGAITNADG | 3 | Best |
| | NO: 59) | NO: 1036) | 127)(197, 209) | (SEQ ID NO: 442) | | |
| | | | | DYNRDGVVDGNER | | |
| | | | | (SEQ ID NO: 443) | | |
| | | | | DSDNDGTLDRRLD | | |
| | | | | (SEQ ID NO: 444) | | |
| t350004 | (SEQ ID | (SEQ ID | (216, 228) | DTNGDGVVNFSDL | 1 | Best |
| | NO: 60) | NO: 1037) | | (SEQ ID NO: 445) | | |
| t349914 | (SEQ ID | (SEQ ID | (947, 959)(972, | DLNRDGRLDPVEL | 2 | Best |
| | NO: 61) | NO: 1038) | 984) | (SEQ ID NO: 446) | | |
| | | | | DTNGDGVADVDEL | | |
| | | | | (SEQ ID NO: 447) | | |
| t349840 | (SEQ ID | (SEQ ID | (133, 145)(85, | DADGDERLTPREF | 3 | Best |
| | NO: 62) | NO: 1039) | 97)(109, 121) | (SEQ ID NO: 448) | | |
| | | | | DRNRDGIITRNEM | | |
| | | | | (SEQ ID NO: 449) | | |
| | | | | DVDGNNLLTFEEW | | |
| | | | | (SEQ ID NO: 450) | | |
| t350033 | (SEQ ID | (SEQ ID | (775,787)(741, | DINHSGTVNFNDF | 2 | Best |
| | NO: 63) | NO: 1040) | 753) | (SEQ ID NO: 451) | | |
| | | | | DLNGDGVVNIADL | | |
| | | | | (SEQ ID NO: 452) | | |
| t349804 | (SEQ ID | (SEQ ID | (142, 154)(262, | DRDADGILSDEEL | 2 | Best |
| | NO: 64) | NO: 1041) | 274) | (SEQ ID NO: 453) | | |
| | | | | DHDGRGSLTAAQL | | |
| | | | | (SEQ ID NO: 454) | | |
| t349913 | (SEQ ID | (SEQ ID | (225, 237) | DRDRDGEISLEEW | 1 | Best |
| | NO: 65) | NO: 1042) | | (SEQ ID NO: 455) | | |
| t349985 | (SEQ ID | (SEQ ID | (715,727)(746, | DTNGDGVVNFVDL | 2 | Best |
| | NO: 66) | NO: 1043) | 758) | (SEQ ID NO: 456) | | |
| | | | | DVDADGDVDFADL | | |
| | | | | (SEQ ID NO: 457) | | |
| t349872 | (SEQ ID | (SEQ ID | (46,58)(71,83) | DFNNDGRNETTSW | 3 | Best |
| | NO: 67) | NO: 1044) | (112, 124) | (SEQ ID NO: 458) | | |
| | | | | DRNGNGTIDAGRE | | |
| | | | | (SEQ ID NO: 459) | | |
| | | | | GGNGDGRINRRDA | | |
| | | | | (SEQ ID NO: 460) | | |
| t349778 | (SEQ ID | (SEQ ID | (73, 85) | DENGDGELSREEL | 1 | Best |
| | NO: 68) | NO: 1045) | | (SEQ ID NO: 461) | | |
| t349823 | (SEQ ID | (SEQ ID | (72, 84)(46, 58) | DADRDGLLSLEEF | 2 | Best |
| | NO: 69) | NO: 1046) | | (SEQ ID NO: 462) | | |
| | | | | DANGDGWVNRDEL | | |
| | | | | (SEQ ID NO: 463) | | |
| t349844 | (SEQ ID | (SEQ ID | (436, 448)(377, | DLDGDGLLDLVLG | 7 | Best |
| | NO: 70) | NO: 1047) | 389)(315, 327) | (SEQ ID NO: 464) | | |
| | | | (256, 268)(182, | DLDGDGDTDLLLA | | |
| | | | 194)(123, 135) | (SEQ ID NO: 465) | | |
| | | | (75, 87) | DIDGDGRLDLVMG | | |
| | | | | (SEQ ID NO: 466) | | |
| | | | | DIDGDGDLDLFWG | | |
| | | | | (SEQ ID NO: 467) | | |
| | | | | IDCNGRLDLFIG | | |
| | | | | (SEQ ID NO: 468) | | |
| | | | | DLNGDGRIDLLAE | | |
| | | | | (SEQ ID NO: 469) | | |
| | | | | DLDNSGRVDLFVQ | | |
| | | | | (SEQ ID NO: 470) | | |
| t349805 | (SEQ ID | (SEQ ID | (52, 64)(87, 99) | DKDADGKLSPEER | 3 | Best |
| | NO: 71) | NO: 1048) | (115, 127) | (SEQ ID NO: 471) | | |
| | | | | DADGNGELSPEER | | |
| | | | | (SEQ ID NO: 472) | | |
| | | | | DANKDGKLDPEER | | |
| | | | | (SEQ ID NO: 473) | | |
| t349853 | (SEQ ID | (SEQ ID | (221,233) | DLDGNGEIDIREW | 1 | hit |
| | NO: 72) | NO: 1049) | | (SEQ ID NO: 474) | | |
| t349828 | (SEQ ID | (SEQ ID | (86, 98) | DLNGDGTISVSEA | 1 | hit |
| | NO: 73) | NO: 1050) | | (SEQ ID NO: 475) | | |
| t350014 | (SEQ ID | (SEQ ID | (51,63)(77, 89) | DKNKDGMISKEEY | 2 | hit |
| | NO: 74) | NO: 1051) | | (SEQ ID NO: 476) | | |
| | | | | DLNGDGKLDKTEF | | |
| | | | | (SEQ ID NO: 477) | | |
| t350031 | (SEQ ID | (SEQ ID | (456, 468)(429, | DCDGDGELTLFDF | 2 | hit |
| | NO: 75) | NO: 1052) | 441) | (SEQ ID NO: 478) | | |
| | | | | DFDGDGELTLFDF | | |
| | | | | (SEQ ID NO: 479) | | |
| t349998 | (SEQ ID | (SEQ ID | (96, 108)(120, | DTDKSGDVSLEEF | 2 | hit |
| | NO: 76) | NO: 1053) | 132) | (SEQ ID NO: 480) | | |
| | | | | DANNDGKLTKDEI | | |
| | | | | (SEQ ID NO: 481) | | |
| t349808 | (SEQ ID | (SEQ ID | (494, 506)(450, | GGNGDGKISQQDA | 2 | hit |
| | NO: 77) | NO: 1054) | 462) | (SEQ ID NO: 482) | | |
| | | | | DRNNNGAIDSGRE | | |
| | | | | (SEQ ID NO: 483) | | |
| t349968 | (SEQ ID | (SEQ ID | (280, 292)(253, | DFDGDGSLTLFDF | 2 | hit |
| | NO: 78) | NO: 1055) | 265) | (SEQ ID NO: 484) | | |
| | | | | DIDGDGSLTLFDF | | |
| | | | | (SEQ ID NO: 485) | | |
| t349918 | (SEQ ID | (SEQ ID | (123, 135)(88, | DANKDGDIERSD- | 2 | hit |
| | NO: 79) | NO: 1056) | 100) | (SEQ ID NO: 486) | | |
| | | | | DRNEDGRVDRKER | | |
| | | | | (SEQ ID NO: 487) | | |
| t349786 | (SEQ ID | (SEQ ID | (61,73)(85,97) | DSDGDGKVSRREF | 2 | hit |
| | NO: 80) | NO: 1057) | | (SEQ ID NO: 488) | | |
| | | | | DLNNDRFLDADEI | | |
| | | | | (SEQ ID NO: 489) | | |
| t350007 | (SEQ ID | (SEQ ID | (89, 101) | DINHDGILTEEEF | 1 | hit |
| | NO: 81) | NO: 1058) | | (SEQ ID NO: 490) | | |
| t349949 | (SEQ ID | (SEQ ID | (135, 147)(102, | DPEGTGSLSESQF | 3 | hit |
| | NO: 82) | NO: 1059) | 114)(32, 44) | (SEQ ID NO: 491) | | |
| | | | | DADGDGSVTQAEY | | |
| | | | | (SEQ ID NO: 492) | | |
| | | | | DTNGDGVVSADEV | | |
| | | | | (SEQ ID NO: 493) | | |
| t349827 | (SEQ ID | (SEQ ID | (97, 109)(72, 84) | DTDDDGLLTREEV | 2 | hit |
| | NO: 83) | NO: 1060) | | (SEQ ID NO: 494) | | |
| | | | | DRNGDGKLSRDEL | | |
| | | | | (SEQ ID NO: 495) | | |
| t349930 | (SEQ ID | (SEQ ID | (127, 139)(103, | DANGDGVVTEVEI | 4 | hit |
| | NO: 84) | NO: 1061) | 115)(46,58)(70, | (SEQ ID NO: 496) | | |
| | | | 82) | DKDGDGRVSAREF | | |
| | | | | (SEQ ID NO: 497) | | |
| | | | | DGNGDGAVTRAEY | | |
| | | | | (SEQ ID NO: 498) | | |
| | | | | DRDGDGYVTQAEI | | |
| | | | | (SEQ ID NO: 499) | | |
| t349790 | (SEQ ID | (SEQ ID | (151, 163)(175, | DADGDGALSMQEF | 2 | hit |
| | NO: 85) | NO: 1062) | 187) | (SEQ ID NO: 500) | | |
| | | | | DGDDDGSLTMEEM | | |
| | | | | (SEQ ID NO: 501) | | |
| t349825 | (SEQ ID | (SEQ ID | (411,423)(441, | DTNGDGVVNFTDL | 2 | hit |
| | NO: 86) | NO: 1063) | 453) | (SEQ ID NO: 502) | | |
| | | | | DVNDDGVVDFSDL | | |
| | | | | (SEQ ID NO: 503) | | |
| t349795 | (SEQ ID | (SEQ ID | (93, 105) | DLNGSGKLSFDEL | 1 | hit |
| | NO: 87) | NO: 1064) | | (SEQ ID NO: 504) | | |
| t350011 | (SEQ ID | (SEQ ID | (26, 38)(59, 71) | DVDGDGEVTLMDV | 2 | hit |
| | NO: 88) | NO: 1065) | | (SEQ ID NO: 505) | | |
| | | | | DLNQDGVVDASDV | | |
| | | | | (SEQ ID NO: 506) | | |
| t349775 | (SEQ ID | (SEQ ID | (25, 37) | DADRDGTVNDEEI | 1 | hit |
| | NO: 89) | NO: 1066) | | (SEQ ID NO: 507) | | |
| t349935 | (SEQ ID | (SEQ ID | (271,283)(244, | DFDGDGSLTIFDF | 2 | hit |
| | NO: 90) | NO: 1067) | 256) | (SEQ ID NO: 508) | | |
| | | | | DIDGDGQLTIFDF | | |
| | | | | (SEQ ID NO: 509) | | |
| t350005 | (SEQ ID | (SEQ ID | (79, 91)(105, | DRDGDGKLSTAEA | 2 | hit |
| | NO: 91) | NO: 1068) | 117) | (SEQ ID NO: 510) | | |
| | | | | DKDGDGALSRTEF | | |
| | | | | (SEQ ID NO: 511) | | |
| t349934 | (SEQ ID | (SEQ ID | (85,97)(59,71) | DSNGDRTLVLEEF | 2 | hit |
| | NO: 92) | NO: 1069) | | (SEQ ID NO: 512) | | |
| | | | | DTDGSQSLSEEEA | | |
| | | | | (SEQ ID NO: 513) | | |
| t349964 | (SEQ ID | (SEQ ID | (131, 143)(185, | DLNADGRLDQVSM | 3 | hit |
| | NO: 93) | NO: 1070) | 197)(154, 166) | (SEQ ID NO: 514) | | |
| | | | | DDNGDGRIDASDA | | |
| | | | | (SEQ ID NO: 515) | | |
| | | | | DRNGNGRIDDGKE | | |
| | | | | (SEQ ID NO: 516) | | |
| t349958 | (SEQ ID | (SEQ ID | (153, 165)(180, | DIDGDGQLTLFDF | 2 | hit |
| | NO: 94) | NO: 1071) | 192) | (SEQ ID NO: 517) | | |
| | | | | DFDGDGQLTLFDF | | |
| | | | | (SEQ ID NO: 518) | | |
| t349888 | (SEQ ID | (SEQ ID | (60, 72)(225, | DLNGDGKLDVVVA | 3 | hit |
| | NO: 95) | NO: 1072) | 237)(323, 335) | (SEQ ID NO: 519) | | |
| | | | | DLNRDGKADIVAT | | |
| | | | | (SEQ ID NO: 520) | | |
| | | | | DLNNDGAIDLVTS | | |
| | | | | (SEQ ID NO: 521) | | |
| t349849 | (SEQ ID | (SEQ ID | (87, 99)(56, 68) | DLNGDGVVNFTDL | 2 | hit |
| | NO: 96) | NO: 1073) | | (SEQ ID NO: 522) | | |
| | | | | DTNGDGVVNFTDL | | |
| | | | | (SEQ ID NO: 523) | | |
| t349988 | (SEQ ID | (SEQ ID | (37, 49)(65, 77) | DVNNDGKVDAADV | 2 | hit |
| | NO: 97) | NO: 1074) | | (SEQ ID NO: 524) | | |
| | | | | DVNKDGKIDAADV | | |
| | | | | (SEQ ID NO: 525) | | |
| t349992 | (SEQ ID | (SEQ ID | (245, 257)(293, | DLDGDGKEEVIFA | 4 | hit |
| | NO: 98) | NO: 1075) | 305)(394, 406) | (SEQ ID NO: 526) | | |
| | | | (466, 478) | DLNKNGKPEIYVS | | |
| | | | | (SEQ ID NO: 527) | | |
| | | | | DLDNDGSREIIAI | | |
| | | | | (SEQ ID NO: 528) | | |
| | | | | DVNGDGQQDIIIN | | |
| | | | | (SEQ ID NO: 529) | | |
| t349822 | (SEQ ID | (SEQ ID | (212, 224)(102, | GLLADGELSVDEV | 2 | hit |
| | NO: 99) | NO: 1076) | 114) | (SEQ ID NO: 530) | | |
| | | | | DADRDGTLTVEEV | | |
| | | | | (SEQ ID NO: 531) | | |
| t349760 | (SEQ ID | (SEQ ID | (839, 851)(812, | DVNRDGVLNFADI | 2 | hit |
| | NO: 100) | NO: 1077) | 824) | (SEQ ID NO: 532) | | |
| | | | | DVNADGVVNAEDL | | |
| | | | | (SEQ ID NO: 533) | | |
| t349991 | (SEQ ID | (SEQ ID | (402, 414)(370, | DINKDGFVNFTDM | 2 | hit |
| | NO: 101) | NO: 1078) | 382) | (SEQ ID NO: 534) | | |
| | | | | DLDYSGTVNFEDL | | |
| | | | | (SEQ ID NO: 535) | | |
| t349890 | (SEQ ID | (SEQ ID | (131, 143) | PFNGDGYIDPEDV | 1 | hit |
| | NO: 102) | NO: 1079) | | (SEQ ID NO: 536) | | |
| t349959 | (SEQ ID | (SEQ ID | (243, 255) | DKNGDNYIPFFNS | 1 | hit |
| | NO: 103) | NO: 1080) | | (SEQ ID NO: 537) | | |
| t349877 | (SEQ ID | (SEQ ID | (137, 149)(113, | DADGDAKLTPAEF | 3 | hit |
| | NO: 104) | NO: 1081) | 125)(89, 101) | (SEQ ID NO: 538) | | |
| | | | | DTNGDNLLSFEEW | | |
| | | | | (SEQ ID NO: 539) | | |
| | | | | DRNRDGVITRIEM | | |
| | | | | (SEQ ID NO: 540) | | |
| t349892 | (SEQ ID | (SEQ ID | (24, 36)(44, 56) | DDDRDGVLSGKEL | 2 | hit |
| | NO: 105) | NO: 1082) | | (SEQ ID NO: 541) | | |
| | | | | DLNGNGLCERGEV | | |
| | | | | (SEQ ID NO: 542) | | |
| t349884 | (SEQ ID | (SEQ ID | (277, 289)(265, | DTNGDGKVTLADW | 2 | hit |
| | NO: 106) | NO: 1083) | 277) | (SEQ ID NO: 543) | | |
| | | | | DVNGDGKITNDID | | |
| | | | | (SEQ ID NO: 544) | | |
| t349955 | (SEQ ID | (SEQ ID | (365, 377)(392, | DINGDGVLNFFDV | 2 | hit |
| | NO: 107) | NO: 1084) | 404) | (SEQ ID NO: 545) | | |
| | | | | DFNGDGAFDFFDI | | |
| | | | | (SEQ ID NO: 546) | | |
| t350012 | (SEQ ID | (SEQ ID | (406,418) | DRDQDGSVSKTEF | 1 | hit |
| | NO: 108) | NO: 1085) | | (SEQ ID NO: 547) | | |
| t349770 | (SEQ ID | (SEQ ID | (99, ill)(125, | DQDKDGQISKGEA | 2 | hit |
| | NO: 109) | NO: 1086) | 137) | (SEQ ID NO: 548) | | |
| | | | | DTDRNGKISLIEW | | |
| | | | | (SEQ ID NO: 549) | | |
| t349847 | (SEQ ID | (SEQ ID | (426, 438) | DFDGDGALTLFDF | 1 | hit |
| | NO: 110) | NO: 1087) | | (SEQ ID NO: 550) | | |
| t349793 | (SEQ ID | (SEQ ID | (63, 75) | DVDGNGYIDYVGS | 1 | hit |
| | NO: 111) | NO: 1088) | | (SEQ ID NO: 551) | | |
| t349788 | (SEQ ID | (SEQ ID | (196, 208)(220, | DQNGDGKVDKTEY | 8 | hit |
| | NO: 112) | NO: 1089) | 232)(148, 160) | (SEQ ID NO: 552) | | |
| | | | (124, 136)(81, | DLDKNGELSSDEY | | |
| | | | 93)(263, 275) | (SEQ ID NO: 553) | | |
| | | | (287, 299)(57, | DTNKDGVISKADP | | |
| | | | 69) | (SEQ ID NO: 554) | | |
| | | | | DKNKSKTIGHDEY | | |
| | | | | (SEQ ID NO: 555) | | |
| | | | | ENKDGQVSVDEY | | |
| | | | | (SEQ ID NO: 556) | | |
| | | | | DTDNSQQISSAEF | | |
| | | | | (SEQ ID NO: 557) | | |
| | | | | DTDLNQQVTMAEA | | |
| | | | | (SEQ ID NO: 558) | | |
| | | | | DLDKDGTVSEAEF | | |
| | | | | (SEQ ID NO: 559) | | |
| t349956 | (SEQ ID | (SEQ ID | (705,717)(680, | DKNGDQLLTRDEL | 2 | hit |
| | NO: 113) | NO: 1090) | 692) | (SEQ ID NO: 560) | | |
| | | | | DRNGDGVVSKAEV | | |
| | | | | (SEQ ID NO: 561) | | |
| t350041 | (SEQ ID | (SEQ ID | (432, 444)(459, | DLDHDGVLTIFDY | 2 | hit |
| | NO: 114) | NO: 1091) | 471) | (SEQ ID NO: 562) | | |
| | | | | DFDGDGELTLFDF | | |
| | | | | (SEQ ID NO: 563) | | |
| t350000 | (SEQ ID | (SEQ ID | (145, 157)(168, | DADGDGELSAMEI | 4 | hit |
| | NO: 115) | NO: 1092) | 180)(220, 232) | (SEQ ID NO: 564) | | |
| | | | (244, 256) | DTNGDGKLSVDEL | | |
| | | | | (SEQ ID NO: 565) | | |
| | | | | DTNNDGFLSGDEI | | |
| | | | | (SEQ ID NO: 566) | | |
| | | | | DKDGDGKISKEEL | | |
| | | | | (SEQ ID NO: 567) | | |
| t349906 | (SEQ ID | (SEQ ID | (222, 234)(257, | DTNRDGELNGEEA | 6 | hit |
| | NO: 116) | NO: 1093) | 269)(124, 136) | (SEQ ID NO: 568) | | |
| | | | (53, 65)(172, | DGNGDGVFDEAEM | | |
| | | | 184)(88, 100) | (SEQ ID NO: 569) | | |
| | | | | DKDGDFRLSADEQ | | |
| | | | | (SEQ ID NO: 570) | | |
| | | | | DLNGDGVVDDEEA | | |
| | | | | (SEQ ID NO: 571) | | |
| | | | | ANGDGIVDEQEA | | |
| | | | | (SEQ ID NO: 572) | | |
| | | | | DANGDGNLDDEEQ | | |
| | | | | (SEQ ID NO: 573) | | |
| t349799 | (SEQ ID | (SEQ ID | (513,525)(543, | DKDGDGTLTRDEF | 2 | hit |
| | NO: 117) | NO: 1094) | 555) | (SEQ ID NO: 574) | | |
| | | | | DADKNGVLSRDEF | | |
| | | | | (SEQ ID NO: 575) | | |
| t349878 | (SEQ ID | (SEQ ID | (140, 152)(216, | DLDGDGMLSLSEF | 4 | hit |
| | NO: 118) | NO: 1095) | 228)(94, 106) | (SEQ ID NO: 576) | | |
| | | | (54, 66) | DTNQDGQISGDEL | | |
| | | | | (SEQ ID NO: 577) | | |
| | | | | DTDASGALDTAEV | | |
| | | | | (SEQ ID NO: 578) | | |
| | | | | DMDTDCSVSQSEF | | |
| | | | | (SEQ ID NO: 579) | | |
| t349922 | (SEQ ID | (SEQ ID | (553,565)(521, | DTDRDGQLTQDEF | 2 | hit |
| | NO: 119) | NO: 1096) | 533) | (SEQ ID NO: 580) | | |
| | | | | DQNHDGVLSHEEF | | |
| | | | | (SEQ ID NO: 581) | | |
| t350003 | (SEQ ID | (SEQ ID | (279, 291)(488, | DADHDGKVTLKEL | 2 | hit |
| | NO: 120) | NO: 1097) | 500) | (SEQ ID NO: 582) | | |
| | | | | DISGDGTVQMLYP | | |
| | | | | (SEQ ID NO: 583) | | |
| t349990 | (SEQ ID | (SEQ ID | (14, 26)(167, | IRANNQVISFAEY | 2 | hit |
| | NO: 121) | NO: 1098) | 179) | (SEQ ID NO: 584) | | |
| | | | | DADNSGTVDPDEI | | |
| | | | | (SEQ ID NO: 585) | | |
| t349950 | (SEQ ID | (SEQ ID | (99, 111)(75, 87) | DANGDGKLSLEEL | 4 | hit |
| | NO: 122) | NO: 1099) | (155, 167)(131, | (SEQ ID NO: 586) | | |
| | | | 143) | DADKDGKLSAAEM | | |
| | | | | (SEQ ID NO: 587) | | |
| | | | | DADGDGALSMEEM | | |
| | | | | (SEQ ID NO: 588) | | |
| | | | | DADGDGMLSAEEG | | |
| | | | | (SEQ ID NO: 589) | | |
| t349791 | (SEQ ID | (SEQ ID | (253, 265) | DRNRDGYVTGTEL | 1 | hit |
| | NO: 123) | NO: 1100) | | (SEQ ID NO: 590) | | |
| t347903 | (SEQ ID | (SEQ ID | | | 0 | hit |
| | NO: 124) | NO: 1101) | | | | |
| t349845 | (SEQ ID | (SEQ ID | (365, 377)(387, | DTNNDGRLTREEY | 2 | hit |
| | NO: 125) | NO: 1102) | 399) | (SEQ ID NO: 591) | | |
| | | | | DANRDGFATPEEV | | |
| | | | | (SEQ ID NO: 592) | | |
| t349812 | (SEQ ID | (SEQ ID | (405, 417)(378, | DLDGDGSLTLFDF | 2 | hit |
| | NO: 126) | NO: 1103) | 390) | (SEQ ID NO: 593) | | |
| | | | | DFDGDGTLTLFDF | | |
| | | | | (SEQ ID NO: 594) | | |
| t350036 | (SEQ ID | (SEQ ID | (139, 151)(112, | DSDASGTLTREEL( | 4 | hit |
| | NO: 127) | NO: 1104) | 124)(50, 62)(77, | SEQ ID NO: 595) | | |
| | | | 89) | DTNGDGVIDAGEA | | |
| | | | | (SEQ ID NO: 596) | | |
| | | | | DTDGDGAISLAEA | | |
| | | | | (SEQ ID NO: 597) | | |
| | | | | DADGDGLVTRAEM | | |
| | | | | (SEQ ID NO: 598) | | |
| t349908 | (SEQ ID | (SEQ ID | (166, 178)(68, | DRNGDGVLSPADF | 4 | hit |
| | NO: 128) | NO: 1105) | 80)(44, 56)(142, | (SEQ ID NO: 599) | | |
| | | | 154) | DADDNGAYSAEEL | | |
| | | | | (SEQ ID NO: 600) | | |
| | | | | DLDGDGAVTLAEA | | |
| | | | | (SEQ ID NO: 601) | | |
| | | | | DANHDGTVTRTEF | | |
| | | | | (SEQ ID NO: 602) | | |
| t349783 | (SEQ ID | (SEQ ID | (243, 255) | DKNGDNYIPFFNS | 1 | hit |
| | NO: 129) | NO: 1106) | | (SEQ ID NO: 603) | | |
| t349873 | (SEQ ID | (SEQ ID | (709, 721)(682, | DIDGDGHLTLFDF | 2 | hit |
| | NO: 130) | NO: 1107) | 694) | (SEQ ID NO: 604) | | |
| | | | | DLDGDATLTLYDM | | |
| | | | | (SEQ ID NO: 605) | | |
| t350006 | (SEQ ID | (SEQ ID | (733, 745)(760, | DINGDGLLNFFDI | 2 | hit |
| | NO: 131) | NO: 1108) | 772) | (SEQ ID NO: 606) | | |
| | | | | DFNNDGLFNFFDF | | |
| | | | | (SEQ ID NO: 607) | | |
| t349870 | (SEQ ID | (SEQ ID | | | 0 | hit |
| | NO: 132) | NO: 1109) | | | | |
| t349868 | (SEQ ID | (SEQ ID | (281,293) | DRNGNGTIDNGAE | 1 | hit |
| | NO: 133) | NO: 1110) | | (SEQ ID NO: 608) | | |
| t349839 | (SEQ ID | (SEQ ID | (121, 133)(165, | DANSDNALSKSEF | 2 | hit |
| | NO: 134) | NO: 1111) | 177) | (SEQ ID NO: 609) | | |
| | | | | DANKDGAVTPAEL | | |
| | | | | (SEQ ID NO: 610) | | |
| t349838 | (SEQ ID | (SEQ ID | (43, 55)(67, 79) | DSDGDGAVSKAEA | 5 | hit |
| | NO: 135) | NO: 1112) | (91, 103)(158, | (SEQ ID NO: 611) | | |
| | | | 170)(182, 194) | DVNKDGQINAADR | | |
| | | | | (SEQ ID NO: 612) | | |
| | | | | DTDKNGSISEAEF | | |
| | | | | (SEQ ID NO: 613) | | |
| | | | | DANGDKTITNAEM | | |
| | | | | (SEQ ID NO: 614) | | |
| | | | | SNKDGKISAEEH | | |
| | | | | (SEQ ID NO: 615) | | |
| t349898 | (SEQ ID | (SEQ ID | (63, 75)(17, 29) | DINGDGLMDFVTG | 3 | hit |
| | NO: 136) | NO: 1113) | (127, 139) | (SEQ ID NO: 616) | | |
| | | | | DGDGDNDVLSSSA | | |
| | | | | (SEQ ID NO: 617) | | |
| | | | | DVNGDGLLDIVTS | | |
| | | | | (SEQ ID NO: 618) | | |
| t350045 | (SEQ ID | (SEQ ID | (183, 195)(124, | DYDDNGVVSLAEL | 2 | hit |
| | NO: 137) | NO: 1114) | 136) | (SEQ ID NO: 619) | | |
| | | | | DVSGDGNLSTAEI | | |
| | | | | (SEQ ID NO: 620) | | |
| t349806 | (SEQ ID | (SEQ ID | (272, 284)(344, | DLDGDGIELTGYA | 3 | hit |
| | NO: 138) | NO: 1115) | 356)(313, 325) | (SEQ ID NO: 621) | | |
| | | | | DSNGDGRIDRLDK | | |
| | | | | (SEQ ID NO: 622) | | |
| | | | | DRNGNGIIDSGKE | | |
| | | | | (SEQ ID NO: 623) | | |
| t349842 | (SEQ ID | (SEQ ID | (149, 161)(119, | DADKSGGIDHNE | 2 | hit |
| | NO: 139) | NO: 1116) | 131) | W(SEQ ID NO: | | |
| | | | | 624) | | |
| | | | | DTNGDAQVVKEEF | | |
| | | | | (SEQ ID NO: 625) | | |
| t349824 | (SEQ ID | (SEQ ID | (85, 97)(50, 62) | DQNGDGELSAGEL | 2 | hit |
| | NO: 140) | NO: 1117) | | (SEQ ID NO: 626) | | |
| | | | | DQDGSNSVSESEY | | |
| | | | | (SEQ ID NO: 627) | | |
| t349857 | (SEQ ID | (SEQ ID | (146, 158)(74, | DSDASGSLSETEY | 4 | hit |
| | NO: 141) | NO: 1118) | 86)(95, 107) | (SEQ ID NO: 628) | | |
| | | | (120, 132) | DSDASGDLSEEEF | | |
| | | | | (SEQ ID NO: 629) | | |
| | | | | DSDGDGAVSEAEF | | |
| | | | | (SEQ ID NO: 630) | | |
| | | | | DTDGSGDLSEDEL | | |
| | | | | (SEQ ID NO: 631) | | |
| t347907 | (SEQ ID | (SEQ ID | | | 0 | hit |
| | NO: 142) | NO: 1119) | | | | |
| t349768 | (SEQ ID | (SEQ ID | (188, 200)(134, | DANRDGRLDAQDA | 3 | hit |
| | NO: 143) | NO: 1120) | 146)(157, 169) | (SEQ ID NO: 632) | | |
| | | | | DLNGDGRQTLTSM | | |
| | | | | (SEQ ID NO: 633) | | |
| | | | | DRNGNGRIDDGRE | | |
| | | | | (SEQ ID NO: 634) | | |
| t350034 | (SEQ ID | (SEQ ID | (121, 133)(183, | DADQDGKLAAEEM | 4 | hit |
| | NO: 144) | NO: 1121) | 195)(97, 109) | (SEQ ID NO: 635) | | |
| | | | (157, 169) | DDNADGAISKEEM | | |
| | | | | (SEQ ID NO: 636) | | |
| | | | | DADQNGQITKEEL | | |
| | | | | (SEQ ID NO: 637) | | |
| | | | | DADQDGFISPAEL | | |
| | | | | (SEQ ID NO: 638) | | |
| t349802 | (SEQ ID | (SEQ ID | (201,213) | DQDRSGKVDMQEW | 1 | hit |
| | NO: 145) | NO: 1122) | | (SEQ ID NO: 639) | | |
| t349941 | (SEQ ID | (SEQ ID | (213, 225)(180, | DVNNDGVIDLRDF | 3 | hit |
| | NO: 146) | NO: 1123) | 192)(86, 98) | (SEQ ID NO: 640) | | |
| | | | | DRDGDGRLDRWQG | | |
| | | | | (SEQ ID NO: 641) | | |
| | | | | DPDRSGAFGITDV | | |
| | | | | (SEQ ID NO: 642) | | |
| t349932 | (SEQ ID | (SEQ ID | (75, 87)(49, 61) | DANTDSQLSLDEY | 2 | hit |
| | NO: 147) | NO: 1124) | | (SEQ ID NO: 643) | | |
| | | | | DSNGDGMVSLEEF | | |
| | | | | (SEQ ID NO: 644) | | |
| t349869 | (SEQ ID | (SEQ ID | (468, 480) | DANGDGVINFADL | 1 | hit |
| | NO: 148) | NO: 1125) | | (SEQ ID NO: 645) | | |
| t349927 | (SEQ ID | (SEQ ID | (62, 74)(85, 97) | DANGDGSLDASEF | 2 | hit |
| | NO: 149) | NO: 1126) | | (SEQ ID NO: 646) | | |
| | | | | DKNGDGKVSQEEM | | |
| | | | | (SEQ ID NO: 647) | | |
| t350039 | (SEQ ID | (SEQ ID | (125, 137)(156, | DIDGNGLVDFSDL | 2 | hit |
| | NO: 150) | NO: 1127) | 168) | (SEQ ID NO: 648) | | |
| | | | | DLDGNEQVDFYDL | | |
| | | | | (SEQ ID NO: 649) | | |
| t349965 | (SEQ ID | (SEQ ID | (225, 237) | DANRDGEISLNEW | 1 | hit |
| | NO: 151) | NO: 1128) | | (SEQ ID NO: 650) | | |
| t349897 | (SEQ ID | (SEQ ID | (589, 601)(562, | DADRDGVVTPADI | 5 | hit |
| | NO: 152) | NO: 1129) | 574)(439, 451) | (SEQ ID NO: 651) | | |
| | | | (321, 333)(143, | DTDRNRRLDFFDI | | |
| | | | 155) | (SEQ ID NO: 652) | | |
| | | | | DFNNDGRVDMFAV | | |
| | | | | (SEQ ID NO: 653) | | |
| | | | | DLDGDGRLDWFMT | | |
| | | | | (SEQ ID NO: 654) | | |
| | | | | FDGDGYLDIYCV | | |
| | | | | (SEQ ID NO: 655) | | |
| t349767 | (SEQ ID | (SEQ ID | (100, 112)(127, | DGNRDGKVSREEL | 2 | hit |
| | NO: 153) | NO: 1130) | 139) | (SEQ ID NO: 656) | | |
| | | | | DADKDGTLTRDEM | | |
| | | | | (SEQ ID NO: 657) | | |
| t349999 | (SEQ ID | (SEQ ID | (96, 108)(120, | DTDKSGDVSLEEF | 2 | hit |
| | NO: 154) | NO: 1131) | 132) | (SEQ ID NO: 658) | | |
| | | | | DANNDGKLTKDEI | | |
| | | | | (SEQ ID NO: 659) | | |
| t349850 | (SEQ ID | (SEQ ID | (400, 412)(434, | DVNCDGKVNLVDF | 2 | hit |
| | NO: 155) | NO: 1132) | 446) | (SEQ ID NO: 660) | | |
| | | | | RLNNDGKVDLVDF | | |
| | | | | (SEQ ID NO: 661) | | |
| t349963 | (SEQ ID | (SEQ ID | (177, 189)(153, | DANKDGTVTPEER | 4 | hit |
| | NO: 156) | NO: 1133) | 165)(66, 78)(90, | (SEQ ID NO: 662) | | |
| | | | 102) | DTNKDGAVSRAEF | | |
| | | | | (SEQ ID NO: 663) | | |
| | | | | DVNKDGKLDQVDR | | |
| | | | | (SEQ ID NO: 664) | | |
| | | | | DTDGNGAISRDEF | | |
| | | | | (SEQ ID NO: 665) | | |
| t349891 | (SEQ ID | (SEQ ID | (448, 460)(405, | DQDRDGNLTPEEI | 3 | hit |
| | NO: 157) | NO: 1134) | 417)(539, 551) | (SEQ ID NO: 666) | | |
| | | | | DRNGDGQVSEEEF | | |
| | | | | (SEQ ID NO: 667) | | |
| | | | | DSNGDGFISPEEF | | |
| | | | | (SEQ ID NO: 668) | | |
| t349830 | (SEQ ID | (SEQ ID | (52, 64)(76, 88) | DTNGDKSISKDEH | 2 | hit |
| | NO: 158) | NO: 1135) | | (SEQ ID NO: 669) | | |
| | | | | DKNGDGVISHEEF | | |
| | | | | (SEQ ID NO: 670) | | |
| t349984 | (SEQ ID | (SEQ ID | (59,71) | DVDGNGYIDFCGS | 1 | hit |
| | NO: 159) | NO: 1136) | | (SEQ ID NO: 671) | | |
| t349848 | (SEQ ID | (SEQ ID | (216, 228) | DTDGDGKIDIDDL | 1 | hit |
| | NO: 160) | NO: 1137) | | (SEQ ID NO: 672) | | |
| t350016 | (SEQ ID | (SEQ ID | (276, 288)(300, | DSNGDGKLTRDEL | 2 | hit |
| | NO: 161) | NO: 1138) | 312) | (SEQ ID NO: 673) | | |
| | | | | DTNQDGAITVEEP | | |
| | | | | (SEQ ID NO: 674) | | |
| t349800 | (SEQ ID | (SEQ ID | (138, 150)(187, | DVDADGRLELTLE | 3 | hit |
| | NO: 162) | NO: 1139) | 199)(106, 118) | (SEQ ID NO: 675) | | |
| | | | | DDDDNNQVSYGEL | | |
| | | | | (SEQ ID NO: 676) | | |
| | | | | DKDGNGYISPQEY | | |
| | | | | (SEQ ID NO: 677) | | |
| t349836 | (SEQ ID | (SEQ ID | (847, 859)(818, | DVNNDGVVNFNDL | 2 | hit |
| | NO: 163) | NO: 1140) | 830) | (SEQ ID NO: 678) | | |
| | | | | DTNGDNVVNFTDL | | |
| | | | | (SEQ ID NO: 679) | | |
| t349860 | (SEQ ID | (SEQ ID | (93, 105)(168, | DKNRDGAIAKDEL | 4 | hit |
| | NO: 164) | NO: 1141) | 180)(254, 266) | (SEQ ID NO: 680) | | |
| | | | (230, 242) | DTNKDGSISREEF | | |
| | | | | (SEQ ID NO: 681) | | |
| | | | | DANKDGRVTAEER | | |
| | | | | (SEQ ID NO: 682) | | |
| | | | | DADKDGHITLAEA | | |
| | | | | (SEQ ID NO: 683) | | |
| t349982 | (SEQ ID | (SEQ ID | (172, 184)(267, | DRNKDGIIQTIVL | 2 | hit |
| | NO: 165) | NO: 1142) | 279) | (SEQ ID NO: 684) | | |
| | | | | DTNGNGRLDREDE | | |
| | | | | (SEQ ID NO: 685) | | |
| t349781 | (SEQ ID | (SEQ ID | (301,313)(328, | DVNGDGSLDLFDF | 2 | hit |
| | NO: 166) | NO: 1143) | 340) | (SEQ ID NO: 686) | | |
| | | | | DFDGDCSLTLFDF | | |
| | | | | (SEQ ID NO: 687) | | |
| t349826 | (SEQ ID | (SEQ ID | (109, 121) | DLDHSRFISKHEA | 1 | hit |
| | NO: 167) | NO: 1144) | | (SEQ ID NO: 688) | | |
| t349792 | (SEQ ID | (SEQ ID | (637, 649)(610, | DLDGDGAVSREEF | 2 | hit |
| | NO: 168) | NO: 1145) | 622) | (SEQ ID NO: 689) | | |
| | | | | DSDGDGTISAEEL | | |
| | | | | (SEQ ID NO: 690) | | |
| t349784 | (SEQ ID | (SEQ ID | (97, 109)(121, | DADGDGKVTPEEM | 3 | hit |
| | NO: 169) | NO: 1146) | 133)(153, 165) | (SEQ ID NO: 691) | | |
| | | | | DGDGDGSLSIAEF | | |
| | | | | (SEQ ID NO: 692) | | |
| | | | | DADGDGAVTAEEM | | |
| | | | | (SEQ ID NO: 693) | | |
| t350042 | (SEQ ID | (SEQ ID | (90, 102)(59, 71) | DTDGDGGITLREA | 2 | hit |
| | NO: 170) | NO: 1147) | | (SEQ ID NO: 694) | | |
| | | | | DYDGNGKLTANEL | | |
| | | | | (SEQ ID NO: 695) | | |
| t349894 | (SEQ ID | (SEQ ID | (221,233) | DLDGNGEIDVREW | 1 | hit |
| | NO: 171) | NO: 1148) | | (SEQ ID NO: 696) | | |
| t349809 | (SEQ ID | (SEQ ID | (45,57)(69,81) | DADGDGRVSLAEY | 3 | hit |
| | NO: 172) | NO: 1149) | (110, 122) | (SEQ ID NO: 697) | | |
| | | | | DRNGDGILQDGEL | | |
| | | | | (SEQ ID NO: 698) | | |
| | | | | DRDGDGHLDAAEL | | |
| | | | | (SEQ ID NO: 699) | | |
| t350017 | (SEQ ID | (SEQ ID | (202, 214)(152, | DTDGDYYLSQIEF | 2 | hit |
| | NO: 173) | NO: 1150) | 164) | (SEQ ID NO: 700) | | |
| | | | | DRNSDGRLSVAEY | | |
| | | | | (SEQ ID NO: 701) | | |
| t349929 | (SEQ ID | (SEQ ID | (46, 58)(175, | KYSKNGYMSRFQF | 2 | hit |
| | NO: 174) | NO: 1151) | 187) | (SEQ ID NO: 702) | | |
| | | | | DQDGDGALSFKEF | | |
| | | | | (SEQ ID NO: 703) | | |
| t349763 | (SEQ ID | (SEQ ID | (572, 584) | DRDGDGRVDGDEW | 1 | hit |
| | NO: 175) | NO: 1152) | | (SEQ ID NO: 704) | | |
| t349978 | (SEQ ID | (SEQ ID | (82, 94)(109, | DNDGDGRITSVGI | 4 | hit |
| | NO: 176) | NO: 1153) | 121)(59, 71)(35, | (SEQ ID NO: 705) | | |
| | | | 47) | DLDGDGYLSPAEA | | |
| | | | | (SEQ ID NO: 706) | | |
| | | | | DVNKDGVIDPDEF | | |
| | | | | (SEQ ID NO: 707) | | |
| | | | | DYDGDGRISRAEF | | |
| | | | | (SEQ ID NO: 708) | | |
| t349841 | (SEQ ID | (SEQ ID | (157, 169) | DRDNDGVLSLDEW | 1 | hit |
| | NO: 177) | NO: 1154) | | (SEQ ID NO: 709) | | |
| t349943 | (SEQ ID | (SEQ ID | (76, 88)(111, | DTDKSGTVTRVEI | 4 | hit |
| | NO: 178) | NO: 1155) | 123)(137, 149) | (SEQ ID NO: 710) | | |
| | | | (50, 62) | DTDNDGSLTREEA | | |
| | | | | (SEQ ID NO: 711) | | |
| | | | | DADKSGAVTGKEI | | |
| | | | | (SEQ ID NO: 712) | | |
| | | | | DKDNDGSLTREEA | | |
| | | | | (SEQ ID NO: 713) | | |
| t349864 | (SEQ ID | (SEQ ID | (417, 429)(390, | DCDEDGELTLFDF | 2 | hit |
| | NO: 179) | NO: 1156) | 402) | (SEQ ID NO: 714) | | |
| | | | | DIDGDGQLTIFDF | | |
| | | | | (SEQ ID NO: 715) | | |
| t349947 | (SEQ ID | (SEQ ID | (775, 787)(807, | DLDGDGEIGFGDL | 2 | Weak |
| | NO: 180) | NO: 1157) | 819) | (SEQ ID NO: 716) | | |
| | | | | DVNGDGVVNFTDL | | |
| | | | | (SEQ ID NO: 717) | | |
| t350025 | (SEQ ID | (SEQ ID | (516, 528)(242, | DADNDGKLELIQA | 2 | Weak |
| | NO: 181) | NO: 1158) | 254) | (SEQ ID NO: 718) | | |
| | | | | DINGDGRLETAIA | | |
| | | | | (SEQ ID NO: 719) | | |
| t349762 | (SEQ ID | (SEQ ID | (29, 41) | DINGDGTVSEEDL | 1 | Weak |
| | NO: 182) | NO: 1159) | | (SEQ ID NO: 720) | | |
| t349900 | (SEQ ID | (SEQ ID | (95, 107)(52, 64) | DKDKDGKLTELEL | 4 | Weak |
| | NO: 183) | NO: 1160) | (138, 150)(181, | (SEQ ID NO: 721) | | |
| | | | 193) | DANGDGYVSKKEF | | |
| | | | | (SEQ ID NO: 722) | | |
| | | | | DTNKDGMLSKQEL | | |
| | | | | (SEQ ID NO: 723) | | |
| | | | | DANGDGQISKEEF | | |
| | | | | (SEQ ID NO: 724) | | |
| t350013 | (SEQ ID | (SEQ ID | | | 0 | Weak |
| | NO: 184) | NO: 1161) | | | | |
| t350026 | (SEQ ID | (SEQ ID | (171, 183)(112, | DYDDNGVVSLSEL | 2 | Weak |
| | NO: 185) | NO: 1162) | 124) | (SEQ ID NO: 725) | | |
| | | | | DVSGDGNLSTAEI | | |
| | | | | (SEQ ID NO: 726) | | |
| t349861 | (SEQ ID | (SEQ ID | (72, 84)(96, 108) | DADADGRITQEEM | 4 | Weak |
| | NO: 186) | NO: 1163) | (132, 144)(157, | (SEQ ID NO: 727) | | |
| | | | 169) | DADGDGALSRDEL | | |
| | | | | (SEQ ID NO: 728) | | |
| | | | | DSDGDGKLSPDEM | | |
| | | | | (SEQ ID NO: 729) | | |
| | | | | DADGDGAITEAEF | | |
| | | | | (SEQ ID NO: 730) | | |
| t349852 | (SEQ ID | (SEQ ID | (244, 256)(167, | DLDGDGKLEVVVG | 2 | Weak |
| | NO: 187) | NO: 1164) | 179) | (SEQ ID NO: 731) | | |
| | | | | DGDGEEEVLISAA | | |
| | | | | (SEQ ID NO: 732) | | |
| t349919 | (SEQ ID | (SEQ ID | (225, 237) | DRDRDGEISLEEW | 1 | Weak |
| | NO: 188) | NO: 1165) | | (SEQ ID NO: 733) | | |
| t349811 | (SEQ ID | (SEQ ID | (381,393)(415, | DTDGDGRITSADL | 2 | Weak |
| | NO: 189) | NO: 1166) | 427) | (SEQ ID NO: 734) | | |
| | | | | DADKDGKVTSADL | | |
| | | | | (SEQ ID NO: 735) | | |
| t349772 | (SEQ ID | (SEQ ID | (167, 179) | DRNKNGRIDKEDF | 1 | Weak |
| | NO: 190) | NO: 1167) | | (SEQ ID NO: 736) | | |
| t350038 | (SEQ ID | (SEQ ID | (110, 122)(154, | DTDGNGSISKEEF | 4 | Weak |
| | NO: 191) | NO: 1168) | 166)(178, 190) | (SEQ ID NO: 737) | | |
| | | | (86, 98) | DTDNDGVITRAEF | | |
| | | | | (SEQ ID NO: 738) | | |
| | | | | DTDGDGTITDAER | | |
| | | | | (SEQ ID NO: 739) | | |
| | | | | DVNDDGQLDKADR | | |
| | | | | (SEQ ID NO: 740) | | |
| t350048 | (SEQ ID | (SEQ ID | (236, 248) | DSDGNGVLDDSEW | 1 | Weak |
| | NO: 192) | NO: 1169) | | (SEQ ID NO: 741) | | |
| t349854 | (SEQ ID | (SEQ ID | (74, 86)(40, 52) | IAGMDGQISEEDW | 2 | Weak |
| | NO: 193) | NO: 1170) | | (SEQ ID NO: 742) | | |
| | | | | DTNADGMISATEF | | |
| | | | | (SEQ ID NO: 743) | | |
| t349780 | (SEQ ID | (SEQ ID | (229, 241) | DRNGDGKIDLEEW | 1 | Weak |
| | NO: 194) | NO: 1171) | | (SEQ ID NO: 744) | | |
| t349814 | (SEQ ID | (SEQ ID | (236, 248) | DDNGNGALDPSEW | 1 | Weak |
| | NO: 195) | NO: 1172) | | (SEQ ID NO: 745) | | |
| t349803 | (SEQ ID | (SEQ ID | (685,697)(661, | DLDRDGSISRQEA | 2 | Weak |
| | NO: 196) | NO: 1173) | 673) | (SEQ ID NO: 746) | | |
| | | | | DTNGDGTLTPDEL | | |
| | | | | (SEQ ID NO: 747) | | |
| t349794 | (SEQ ID | (SEQ ID | (70, 82)(209, | DFDGDGSLTLFDF | 3 | Weak |
| | NO: 197) | NO: 1174) | 221)(43, 55) | (SEQ ID NO: 748) | | |
| | | | | DTNGNGELDAGDL | | |
| | | | | (SEQ ID NO: 749) | | |
| | | | | DLDGDGALTLFDF | | |
| | | | | (SEQ ID NO: 750) | | |
| t350008 | (SEQ ID | (SEQ ID | (66, 78)(102, | DKNADRMISLDEF | 3 | Weak |
| | NO: 198) | NO: 1175) | 114)(134, 146) | (SEQ ID NO: 751) | | |
| | | | | DSDRSGGLDTVEF | | |
| | | | | (SEQ ID NO: 752) | | |
| | | | | DSNHDKKLDFAEY | | |
| | | | | (SEQ ID NO: 753) | | |
| t349951 | (SEQ ID | (SEQ ID | (231,243) | DEDRDGNLDPQEW | 1 | Weak |
| | NO: 199) | NO: 1176) | | (SEQ ID NO: 754) | | |
| t349831 | (SEQ ID | (SEQ ID | (24, 36) | NKDNNGYLTPSEF | 1 | Weak |
| | NO: 200) | NO: 1177) | | (SEQ ID NO: 755) | | |
| t349946 | (SEQ ID | (SEQ ID | (483, 495)(506, | DRDGDGKVSSDEF | 2 | Weak |
| | NO: 201) | NO: 1178) | 518) | (SEQ ID NO: 756) | | |
| | | | | DRNRDGFISADEA | | |
| | | | | (SEQ ID NO: 757) | | |
| t349782 | (SEQ ID | (SEQ ID | (141, 153)(203, | DADGNGKLTRDEI | 4 | Weak |
| | NO: 202) | NO: 1179) | 215)(177, 189) | (SEQ ID NO: 758) | | |
| | | | (117, 129) | DRDGDGNVGTDEI | | |
| | | | | (SEQ ID NO: 759) | | |
| | | | | DADGDGAIGATEF | | |
| | | | | (SEQ ID NO: 760) | | |
| | | | | DADSNGAVSREEL | | |
| | | | | (SEQ ID NO: 761) | | |
| t350028 | (SEQ ID | (SEQ ID | (43, 55)(13, 25) | DRDCSGSISYTEL | 2 | Weak |
| | NO: 203) | NO: 1180) | | (SEQ ID NO: 762) | | |
| | | | | DKTKSGRIDVYGF | | |
| | | | | (SEQ ID NO: 763) | | |
| t349993 | (SEQ ID | (SEQ ID | (331,343) | DTDNNGLISLSEL | 1 | Weak |
| | NO: 204) | NO: 1181) | | (SEQ ID NO: 764) | | |
| t349970 | (SEQ ID | (SEQ ID | (81,93) | DTDQDGRITPLEL | 1 | Weak |
| | NO: 205) | NO: 1182) | | (SEQ ID NO: 765) | | |
| t349863 | (SEQ ID | (SEQ ID | (94, 106)(132, | DVNGDGVVDILDF | 2 | Weak |
| | NO: 206) | NO: 1183) | 144) | (SEQ ID NO: 766) | | |
| | | | | DLDGDGGKSTGQF | | |
| | | | | (SEQ ID NO: 767) | | |
| t349975 | (SEQ ID | (SEQ ID | (100, 112)(127, | DGNRDGKVSREEL | 2 | Weak |
| | NO: 207) | NO: 1184) | 139) | (SEQ ID NO: 768) | | |
| | | | | DADKDGTLTRDEM | | |
| | | | | (SEQ ID NO: 769) | | |
| t349926 | (SEQ ID | (SEQ ID | (46, 58)(70, 82) | DTNGDGNVSKAEF | 4 | Weak |
| | NO: 208) | NO: 1185) | (102, 114)(126, | (SEQ ID NO: 770) | | |
| | | | 138) | DADRSGSLTAAEF | | |
| | | | | (SEQ ID NO: 771) | | |
| | | | | DTDGNGVISQAEW | | |
| | | | | (SEQ ID NO: 772) | | |
| | | | | DKNSDGVLTSTER | | |
| | | | | (SEQ ID NO: 773) | | |
| t349810 | (SEQ ID | (SEQ ID | | | 0 | Weak |
| | NO: 209) | NO: 1186) | | | | |
| t350018 | (SEQ ID | (SEQ ID | (216, 228)(128, | DLNGDGVVSAEEM | 5 | Weak |
| | NO: 210) | NO: 1187) | 140)(89, 101) | (SEQ ID NO: 774) | | |
| | | | (66, 78)(151, | DADGDGKISRAES | | |
| | | | 163) | (SEQ ID NO: 775) | | |
| | | | | DTNGDGVVSPEEL | | |
| | | | | (SEQ ID NO: 776) | | |
| | | | | DGNGDGALSASEL | | |
| | | | | (SEQ ID NO: 777) | | |
| | | | | KDGDGFLTEDEL | | |
| | | | | (SEQ ID NO: 778) | | |
| t349896 | (SEQ ID | (SEQ ID | (236, 248) | DHDGDGALDNKEW | 1 | Weak |
| | NO: 211) | NO: 1188) | | (SEQ ID NO: 779) | | |
| t349966 | (SEQ ID | (SEQ ID | (67, 79)(88, 100) | DENKDGILEMKEV | 3 | Weak |
| | NO: 212) | NO: 1189) | (167, 179) | (SEQ ID NO: 780) | | |
| | | | | DRDGNGVIDNQEF | | |
| | | | | (SEQ ID NO: 781) | | |
| | | | | DMTHDNLLDHEEW | | |
| | | | | (SEQ ID NO: 782) | | |
| t349876 | (SEQ ID | (SEQ ID | (68, 80)(86, 98) | DKNHDGKVSIGER | 3 | Weak |
| | NO: 213) | NO: 1190) | (114, 126) | (SEQ ID NO: 783) | | |
| | | | | DRDKDGDVDFDDF | | |
| | | | | (SEQ ID NO: 784) | | |
| | | | | DTNGDGKVEFDEI | | |
| | | | | (SEQ ID NO: 785) | | |
| t349911 | (SEQ ID | (SEQ ID | (230, 242)(203, | DDDGDGTIDEEEC | 2 | Weak |
| | NO: 214) | NO: 1191) | 215) | (SEQ ID NO: 786) | | |
| | | | | DDDGDGMIDENCQ | | |
| | | | | (SEQ ID NO: 787) | | |
| t350023 | (SEQ ID | (SEQ ID | (105, 117)(134, | DTDGSGMIDRTEA | 2 | Weak |
| | NO: 215) | NO: 1192) | 146) | (SEQ ID NO: 788) | | |
| | | | | DPNGIGPDEFSQA | | |
| | | | | (SEQ ID NO: 789) | | |
| t349837 | (SEQ ID | (SEQ ID | (209, 221)(258, | DKNRDGFLTRAEM | 8 | Weak |
| | NO: 216) | NO: 1193) | 270)(235, 247) | (SEQ ID NO: 790) | | |
| | | | (186, 198)(154, | DANKDGRLSQEEF | | |
| | | | 166)(131, 143) | (SEQ ID NO: 791) | | |
| | | | (81,93)(58, 70) | DINGDGNISLQEW | | |
| | | | | (SEQ ID NO: 792) | | |
| | | | | DANNDGRVSRSEW | | |
| | | | | (SEQ ID NO: 793) | | |
| | | | | HDHNGWLSRGEF | | |
| | | | | (SEQ ID NO: 794) | | |
| | | | | DHNRDNRITRDEW | | |
| | | | | (SEQ ID NO: 795) | | |
| | | | | DWNRDGMLSGDEL | | |
| | | | | (SEQ ID NO: 796) | | |
| | | | | DTNHDGILQRSEW | | |
| | | | | (SEQ ID NO: 797) | | |
| t349923 | (SEQ ID | (SEQ ID | (74, 86)(131, | DRNGDGYFSKDDL | 4 | Weak |
| | NO: 217) | NO: 1194) | 143)(50, 62) | (SEQ ID NO: 798) | | |
| | | | (107, 119) | DSNRDNQVSKAEL | | |
| | | | | (SEQ ID NO: 799) | | |
| | | | | DRNGDGAVSRAEF | | |
| | | | | (SEQ ID NO: 800) | | |
| | | | | DKNRDGRISRAEF | | |
| | | | | (SEQ ID NO: 801) | | |
| t347906 | (SEQ ID | (SEQ ID | | | 0 | Weak |
| | NO: 218) | NO: 1195) | | | | |
| t349976 | (SEQ ID | (SEQ ID | (201,213)(228, | DLDGDGELTLFDF | 2 | Weak |
| | NO: 219) | NO: 1196) | 240) | (SEQ ID NO: 802) | | |
| | | | | DFDRDGDLTLFDF | | |
| | | | | (SEQ ID NO: 803) | | |
| t349883 | (SEQ ID | (SEQ ID | (22, 34)(94, 106) | DRDSDGKLEADDL | 3 | Weak |
| | NO: 220) | NO: 1197) | (130, 142) | (SEQ ID NO: 804) | | |
| | | | | DKEGKGMIPSGEL | | |
| | | | | (SEQ ID NO: 805) | | |
| | | | | DPTGSGTVDYEQF | | |
| | | | | (SEQ ID NO: 806) | | |
| t349886 | (SEQ ID | (SEQ ID | (182, 194)(108, | DRDGDGALTAADA | 6 | Weak |
| | NO: 221) | NO: 1198) | 120)(80, 92) | (SEQ ID NO: 807) | | |
| | | | (253, 265)(154, | DRNGDGHIDRQDR | | |
| | | | 166)(281, 293) | (SEQ ID NO: 808) | | |
| | | | | DTNQDRQVSAEEF | | |
| | | | | (SEQ ID NO: 809) | | |
| | | | | DVNKDGTVTFEEA | | |
| | | | | (SEQ ID NO: 810) | | |
| | | | | ADGDGRVTQAEV | | |
| | | | | (SEQ ID NO: 811) | | |
| | | | | DKNGDGVLTKADR | | |
| | | | | (SEQ ID NO: 812) | | |
| t349974 | (SEQ ID | (SEQ ID | (592, 604)(621, | DVNGDGVVNFTDL | 2 | Weak |
| | NO: 222) | NO: 1199) | 633) | (SEQ ID NO: 813) | | |
| | | | | DTDGDGDVDFEDL | | |
| | | | | (SEQ ID NO: 814) | | |
| t349813 | (SEQ ID | (SEQ ID | (82, 94)(50, 62) | DINRDGRIDSRDI | 2 | Weak |
| | NO: 223) | NO: 1200) | | (SEQ ID NO: 815) | | |
| | | | | DVNGDGVVDTYDL | | |
| | | | | (SEQ ID NO: 816) | | |
| t349969 | (SEQ ID | (SEQ ID | (107, 119) | DLDHSRFISKQEA | 1 | Weak |
| | NO: 224) | NO: 1201) | | (SEQ ID NO: 817) | | |
| t349759 | (SEQ ID | (SEQ ID | (205, 217)(129, | DSDSDGTVDNAEE | 2 | Weak |
| | NO: 225) | NO: 1202) | 141) | (SEQ ID NO: 818) | | |
| | | | | DLDQDGVVDANEL | | |
| | | | | (SEQ ID NO: 819) | | |
| t349862 | (SEQ ID | (SEQ ID | (153, 165)(72, | DANKDGVVTRDEM | 4 | Weak |
| | NO: 226) | NO: 1203) | 84)(177, 189) | (SEQ ID NO: 820) | | |
| | | | (96, 108) | DTNKDGKLDEADR | | |
| | | | | (SEQ ID NO: 821) | | |
| | | | | DANKDGKLTPEER | | |
| | | | | (SEQ ID NO: 822) | | |
| | | | | DANKDGNISRDEF | | |
| | | | | (SEQ ID NO: 823) | | |
| t349912 | (SEQ ID | (SEQ ID | (574, 586)(600, | DTNTDGKISREEA | 2 | Weak |
| | NO: 227) | NO: 1204) | 612) | (SEQ ID NO: 824) | | |
| | | | | DLNKDGFMDKGEL | | |
| | | | | (SEQ ID NO: 825) | | |
| t349774 | (SEQ ID | (SEQ ID | (245, 257) | KLNQSGFIDFEEL | 1 | Weak |
| | NO: 228) | NO: 1205) | | (SEQ ID NO: 826) | | |
| t349879 | (SEQ ID | (SEQ ID | (98, 110)(74, 86) | DKDRDGRLTPREF | 3 | Weak |
| | NO: 229) | NO: 1206) | (50, 62) | (SEQ ID NO: 827) | | |
| | | | | DADGNNLLTFEEW | | |
| | | | | (SEQ ID NO: 828) | | |
| | | | | DRNRDRLITRNEM | | |
| | | | | (SEQ ID NO: 829) | | |
| t349885 | (SEQ ID | (SEQ ID | (46, 58)(70, 82) | DINGDGVVSKEEF | 4 | Weak |
| | NO: 230) | NO: 1207) | (102, 114)(126, | (SEQ ID NO: 830) | | |
| | | | 138) | DADASGSVSQSEL | | |
| | | | | (SEQ ID NO: 831) | | |
| | | | | DADGNGAISEDEW | | |
| | | | | (SEQ ID NO: 832) | | |
| | | | | DKNKDGVLSADER | | |
| | | | | (SEQ ID NO: 833) | | |
| t349858 | (SEQ ID | (SEQ ID | (48, 60)(83, 95) | CPTGHLTVDEFKK | 3 | Weak |
| | NO: 231) | NO: 1208) | (119, 131) | (SEQ ID NO: 834) | | |
| | | | | DTNGDGTIDFREF | | |
| | | | | (SEQ ID NO: 835) | | |
| | | | | DLDGNGYISRSEM | | |
| | | | | (SEQ ID NO: 836) | | |
| t349987 | (SEQ ID | (SEQ ID | (98, 110)(122, | DRDRNDRISRVEL | 2 | Weak |
| | NO: 232) | NO: 1209) | 134) | (SEQ ID NO: 837) | | |
| | | | | DKDGNNLLTFEEW | | |
| | | | | (SEQ ID NO: 838) | | |
| t349977 | (SEQ ID | (SEQ ID | (568, 580)(539, | DLDLDGDVDFTDL | 2 | Weak |
| | NO: 233) | NO: 1210) | 551) | (SEQ ID NO: 839) | | |
| | | | | DVNNDGLVDFSDL | | |
| | | | | (SEQ ID NO: 840) | | |
| t349925 | (SEQ ID | (SEQ ID | (99, 111)(40, 52) | DRNGDGRLTAEEI | 4 | Weak |
| | NO: 234) | NO: 1211) | (16, 28)(75, 87) | (SEQ ID NO: 841) | | |
| | | | | DADRDGVLTRAEV | | |
| | | | | (SEQ ID NO: 842) | | |
| | | | | DTNGDGIVDRGEM | | |
| | | | | (SEQ ID NO: 843) | | |
| | | | | DTNRDGRVTRAEF | | |
| | | | | (SEQ ID NO: 844) | | |
| t349960 | (SEQ ID | (SEQ ID | (219, 231) | DLDGDGEIDLKEW | 1 | Weak |
| | NO: 235) | NO: 1212) | | (SEQ ID NO: 845) | | |
| t349995 | (SEQ ID | (SEQ ID | (426, 438)(453, | DLDHDGVLTIFDY | 2 | Weak |
| | NO: 236) | NO: 1213) | 465) | (SEQ ID NO: 846) | | |
| | | | | DFDGDGELTLFDF | | |
| | | | | (SEQ ID NO: 847) | | |
| t349920 | (SEQ ID | (SEQ ID | (130, 142)(104, | DKDKNGSLSRTEF | 2 | Weak |
| | NO: 237) | NO: 1214) | 116) | (SEQ ID NO: 848) | | |
| | | | | DANRDGQLSSAEA | | |
| | | | | (SEQ ID NO: 849) | | |
| t349997 | (SEQ ID | (SEQ ID | (141, 153)(202, | DTDGDGQLSRDEI | 3 | Weak |
| | NO: 238) | NO: 1215) | 214)(177, 189) | (SEQ ID NO: 850) | | |
| | | | | DADGNGAISPEEF | | |
| | | | | (SEQ ID NO: 851) | | |
| | | | | DGDGDGMVSIEEL | | |
| | | | | (SEQ ID NO: 852) | | |
| t349907 | (SEQ ID | (SEQ ID | (247, 259) | DVDKDGYLTGSEL | 1 | Weak |
| | NO: 239) | NO: 1216) | | (SEQ ID NO: 853) | | |
| t349771 | (SEQ ID | (SEQ ID | (539, 551)(568, | DVNNDGLVDFSDL | 2 | Weak |
| | NO: 240) | NO: 1217) | 580) | (SEQ ID NO: 854) | | |
| | | | | DLDLDGDVDFTDL | | |
| | | | | (SEQ ID NO: 855) | | |
| t349899 | (SEQ ID | (SEQ ID | (407, 419)(432, | DADGNGKVSRDEY | 4 | Weak |
| | NO: 241) | NO: 1218) | 444)(458, 470) | (SEQ ID NO: 856) | | |
| | | | (488, 500) | EKDGDGYISQDEF | | |
| | | | | (SEQ ID NO: 857) | | |
| | | | | NSSGSGKISLAEA | | |
| | | | | (SEQ ID NO: 858) | | |
| | | | | DKNHSGYVTWDEF | | |
| | | | | (SEQ ID NO: 859) | | |
| t349816 | (SEQ ID | (SEQ ID | (27, 39) | DLDGDGALSMFEL | 1 | Weak |
| | NO: 242) | NO: 1219) | | (SEQ ID NO: 860) | | |
| t349945 | (SEQ ID | (SEQ ID | (379, 391) | DKDGDGKITSNRE | 1 | Weak |
| | NO: 243) | NO: 1220) | | (SEQ ID NO: 861) | | |
| t349902 | (SEQ ID | (SEQ ID | (80, 92)(107, | DTNKDGKISKAEY | 2 | Weak |
| | NO: 244) | NO: 1221) | 119) | (SEQ ID NO: 862) | | |
| | | | | DTNKDGFISAAEM | | |
| | | | | (SEQ ID NO: 863) | | |
| t350027 | (SEQ ID | (SEQ ID | (236, 248) | DRDGDGVVSVAEF | 1 | Weak |
| | NO: 245) | NO: 1222) | | (SEQ ID NO: 864) | | |
| t349994 | (SEQ ID | (SEQ ID | (222, 234) | DTNGDNFVSLAEL | 1 | Weak |
| | NO: 246) | NO: 1223) | | (SEQ ID NO: 865) | | |
| t349938 | (SEQ ID | (SEQ ID | (130, 142) | DVDQNGNIDYSQL | 1 | Weak |
| | NO: 247) | NO: 1224) | | (SEQ ID NO: 866) | | |
| t349859 | (SEQ ID | (SEQ ID | (202, 214)(146, | DSDKDGVVTVAEM | 4 | Weak |
| | NO: 248) | NO: 1225) | 158)(110, 122) | (SEQ ID NO: 867) | | |
| | | | (178, 190) | DADRNGQLSAAEF | | |
| | | | | (SEQ ID NO: 868) | | |
| | | | | DGDKNGKVTRAEI | | |
| | | | | (SEQ ID NO: 869) | | |
| | | | | DTNKDGAVSLIEH | | |
| | | | | (SEQ ID NO: 870) | | |
| t349787 | (SEQ ID | (SEQ ID | (145, 157)(247, | DVNGDGVINFADL | 2 | Weak |
| | NO: 249) | NO: 1226) | 259) | (SEQ ID NO: 871) | | |
| | | | | DFDNDGVINFGDL | | |
| | | | | (SEQ ID NO: 872) | | |
| t350001 | (SEQ ID | (SEQ ID | (114, 126)(90, | DSDRDGRITTIEY | 2 | Weak |
| | NO: 250) | NO: 1227) | 102) | (SEQ ID NO: 873) | | |
| | | | | DSNRDGWLTLYEA | | |
| | | | | (SEQ ID NO: 874) | | |
| t349921 | (SEQ ID | (SEQ ID | | | 0 | Weak |
| | NO: 251) | NO: 1228) | | | | |
| t350019 | (SEQ ID | (SEQ ID | (107, 119)(72, | DKNGDGKLDDAER | 2 | Weak |
| | NO: 252) | NO: 1229) | 84) | (SEQ ID NO: 875) | | |
| | | | | DKDGDGKLSDAER | | |
| | | | | (SEQ ID NO: 876) | | |
| t350002 | (SEQ ID | (SEQ ID | (602,614)(626, | DADRNGQLTRDEF | 3 | Weak |
| | NO: 253) | NO: 1230) | 638)(111, 123) | (SEQ ID NO: 877) | | |
| | | | | DLSKTGQLTDEQL | | |
| | | | | (SEQ ID NO: 878) | | |
| | | | | DANQDGKLARDEF | | |
| | | | | (SEQ ID NO: 879) | | |
| t349832 | (SEQ ID | (SEQ ID | (169, 181)(766, | DANQDARLSEEEF | 3 | Weak |
| | NO: 254) | NO: 1231) | 778)(742, 754) | (SEQ ID NO: 880) | | |
| | | | | DTDETGVLTEQQL | | |
| | | | | (SEQ ID NO: 881) | | |
| | | | | DEDQDGQVTREEF | | |
| | | | | (SEQ ID NO: 882) | | |
| t347904 | (SEQ ID | (SEQ ID | | | 0 | Weak |
| | NO: 255) | NO: 1232) | | | | |
| t349971 | (SEQ ID | (SEQ ID | (152, 164)(55, | DLDGSGRVSVEEA | 2 | Weak |
| | NO: 256) | NO: 1233) | 67) | (SEQ ID NO: 883) | | |
| | | | | DRDGDGEVTDEGL | | |
| | | | | (SEQ ID NO: 884) | | |
| t349834 | (SEQ ID | (SEQ ID | (260, 272)(165, | DLNQDGVIDRSTE | 2 | Weak |
| | NO: 257) | NO: 1234) | 177) | (SEQ ID NO: 885) | | |
| | | | | DKNQDGIIQAVIL | | |
| | | | | (SEQ ID NO: 886) | | |
| t350024 | (SEQ ID | (SEQ ID | (527, 539)(606, | DASGDGKLDGDEL | 3 | Weak |
| | NO: 258) | NO: 1235) | 618)(630, 642) | (SEQ ID NO: 887) | | |
| | | | | DRNLDGQLAQPEL | | |
| | | | | (SEQ ID NO: 888) | | |
| | | | | DMDDDGRLNSMEF | | |
| | | | | (SEQ ID NO: 889) | | |
| t349856 | (SEQ ID | (SEQ ID | (107, 119)(74, | DVDRDGKIGLPEV | 2 | Weak |
| | NO: 259) | NO: 1236) | 86) | (SEQ ID NO: 890) | | |
| | | | | DFDGSGAADLADA | | |
| | | | | (SEQ ID NO: 891) | | |
| t349801 | (SEQ ID | (SEQ ID | (102, 114)(62, | DSNEDGYYSKAEA | 4 | Weak |
| | NO: 260) | NO: 1237) | 74)(31, 43)(139, | (SEQ ID NO: 892) | | |
| | | | 151) | DVDGDNRLSREEY | | |
| | | | | (SEQ ID NO: 893) | | |
| | | | | DRDGDGFITLQEG | | |
| | | | | (SEQ ID NO: 894) | | |
| | | | | DLDGNRVYDETEF | | |
| | | | | (SEQ ID NO: 895) | | |
| t349924 | (SEQ ID | (SEQ ID | (138, 150)(114, | DADKSGDLTRAEF | 3 | Weak |
| | NO: 261) | NO: 1238) | 126)(90, 102) | (SEQ ID NO: 896) | | |
| | | | | DKDGNNLLTFEEW | | |
| | | | | (SEQ ID NO: 897) | | |
| | | | | DRDRNDRISRVEM | | |
| | | | | (SEQ ID NO: 898) | | |
| t349851 | (SEQ ID | (SEQ ID | (129, 141)(105, | DADKNRALSPAEY | 3 | Weak |
| | NO: 262) | NO: 1239) | 117)(81,93) | (SEQ ID NO: 899) | | |
| | | | | DLNQDGKLSFEEW | | |
| | | | | (SEQ ID NO: 900) | | |
| | | | | DKDENGRITLAEL | | |
| | | | | (SEQ ID NO: 901) | | |
| t350047 | (SEQ ID | (SEQ ID | (265, 277) | DQDGNGKISPMEL | 1 | Weak |
| | NO: 263) | NO: 1240) | | (SEQ ID NO: 902) | | |
| t350044 | (SEQ ID | (SEQ ID | (86, 98)(76, 88) | DFDGDGDVDAADL | 2 | Weak |
| | NO: 264) | NO: 1241) | | (SEQ ID NO: 903) | | |
| | | | | DLDGDGRLDLDFD | | |
| | | | | (SEQ ID NO: 904) | | |
| t349777 | (SEQ ID | (SEQ ID | (268, 280)(107, | DKNGDGVVSISEA | 2 | Weak |
| | NO: 265) | NO: 1242) | 119) | (SEQ ID NO: 905) | | |
| | | | | KEDKKGRVTRREI | | |
| | | | | (SEQ ID NO: 906) | | |
| t349773 | (SEQ ID | (SEQ ID | (101, 113)(60, | DKDKDGSLSRAEA | 3 | Weak |
| | NO: 266) | NO: 1243) | 72)(125, 137) | (SEQ ID NO: 907) | | |
| | | | | DANKDGKISAEEF | | |
| | | | | (SEQ ID NO: 908) | | |
| | | | | DANNDGVLTLEEL | | |
| | | | | (SEQ ID NO: 909) | | |
| t349796 | (SEQ ID | (SEQ ID | (90, 102)(116, | DTNKNGRIEKDEY | 2 | Weak |
| | NO: 267) | NO: 1244) | 128) | (SEQ ID NO: 910) | | |
| | | | | NKGYCTFEEVAEG | | |
| | | | | (SEQ ID NO: 911) | | |
| t350010 | (SEQ ID | (SEQ ID | (131, 143) | DVDNDGLISIEDL | 1 | Weak |
| | NO: 268) | NO: 1245) | | (SEQ ID NO: 912) | | |
| t349962 | (SEQ ID | (SEQ ID | (255, 267) | DTDNDGKVDVAE | 1 | Weak |
| | NO: 269) | NO: 1246) | | L(SEQID NO: 913) | | |
| t349875 | (SEQ ID | (SEQ ID | (29, 41) | DVNNDGIISDEEL | 1 | Weak |
| | NO: 270) | NO: 1247) | | (SEQ ID NO: 914) | | |
| t349881 | (SEQ ID | (SEQ ID | (183, 195)(157, | DDNADGAISKEEM | 4 | Weak |
| | NO: 271) | NO: 1248) | 169)(121, 133) | (SEQ ID NO: 915) | | |
| | | | (97, 109) | DADQDGFISPAEL | | |
| | | | | (SEQ ID NO: 916) | | |
| | | | | DADQDGKLSAEEM | | |
| | | | | (SEQ ID NO: 917) | | |
| | | | | DADQNGQITKEEL | | |
| | | | | (SEQ ID NO: 918) | | |
| t349940 | (SEQ ID | (SEQ ID | (226, 238)(128, | DADKNGTVTMEER | 3 | Weak |
| | NO: 272) | NO: 1249) | 140)(152, 164) | (SEQ ID NO: 919) | | |
| | | | | DANKDGRVDAADR | | |
| | | | | (SEQ ID NO: 920) | | |
| | | | | DADRNGQISREEF | | |
| | | | | (SEQ ID NO: 921) | | |
| t349948 | (SEQ ID | (SEQ ID | (118, 130)(146, | DANKDGAITKEEY | 2 | Weak |
| | NO: 273) | NO: 1250) | 158) | (SEQ ID NO: 922) | | |
| | | | | DANSDGNLDKAEQ | | |
| | | | | (SEQ ID NO: 923) | | |
| t349882 | (SEQ ID | (SEQ ID | (235, 247)(140, | DRDADGLIQVSIQ | 4 | Weak |
| | NO: 274) | NO: 1251) | 152)(170, 182) | (SEQ ID NO: 924) | | |
| | | | (205, 217) | DSDTTGKLGFEEF | | |
| | | | | (SEQ ID NO: 925) | | |
| | | | | DRDQSGFLGSSQL | | |
| | | | | (SEQ ID NO: 926) | | |
| | | | | YAEEDGSMDFNNF | | |
| | | | | (SEQ ID NO: 927) | | |
| t349996 | (SEQ ID | (SEQ ID | (69, 81)(152, | DSNRDGFVSQAEA | 5 | Weak |
| | NO: 275) | NO: 1252) | 164)(110, 122) | (SEQ ID NO: 928) | | |
| | | | (198, 210)(222, | DKNRDSIITRAEF | | |
| | | | 234) | (SEQ ID NO: 929) | | |
| | | | | DANKDGSITRAEA | | |
| | | | | (SEQ ID NO: 930) | | |
| | | | | DANKDGRVSLAEA | | |
| | | | | (SEQ ID NO: 931) | | |
| | | | | LNRDGQVTPDER | | |
| | | | | (SEQ ID NO: 932) | | |
| t349967 | (SEQ ID | (SEQ ID | (100, 112)(127, | DTNGDGLISRAEA | 2 | Weak |
| | NO: 276) | NO: 1253) | 139) | (SEQ ID NO: 933) | | |
| | | | | DANKDGQLSREEM | | |
| | | | | (SEQ ID NO: 934) | | |
| t350046 | (SEQ ID | (SEQ ID | (211,223)(270, | DSDNNSRLTRAEI | 2 | Weak |
| | NO: 277) | NO: 1254) | 282) | (SEQ ID NO: 935) | | |
| | | | | DANTSGDLDYNEI | | |
| | | | | (SEQ ID NO: 936) | | |
| t349776 | (SEQ ID | (SEQ ID | (417, 429)(390, | DCDEDGELTLFDF | 2 | Weak |
| | NO: 278) | NO: 1255) | 402) | (SEQ ID NO: 937) | | |
| | | | | DIDGDGQLTIFDF | | |
| | | | | (SEQ ID NO: 938) | | |
| t349779 | (SEQ ID | (SEQ ID | (160, 172) | DVNRDGQITVGEI | 1 | Weak |
| | NO: 279) | NO: 1256) | | (SEQ ID NO: 939) | | |
| t350020 | (SEQ ID | (SEQ ID | (133, 145)(69, | DANRDGVITRAEI | 4 | Weak |
| | NO: 280) | NO: 1257) | 81)(94, 106) | (SEQ ID NO: 940) | | |
| | | | (157, 169) | DLNRDGKVTQAEF | | |
| | | | | (SEQ ID NO: 941) | | |
| | | | | DKNRDGQITRAEF | | |
| | | | | (SEQ ID NO: 942) | | |
| | | | | DADRDGILDRAEL | | |
| | | | | (SEQ ID NO: 943) | | |
| t349952 | (SEQ ID | (SEQ ID | (26, 38)(76, 88) | DRNQDGALDEEEF | 3 | Weak |
| | NO: 281) | NO: 1258) | (52, 64) | (SEQ ID NO: 944) | | |
| | | | | DANGDGRLDAEEI | | |
| | | | | (SEQ ID NO: 945) | | |
| | | | | DLDRNGRIDLDEL | | |
| | | | | (SEQ ID NO: 946) | | |
| t350015 | (SEQ ID | (SEQ ID | (127, 139)(100, | DADKDGTLTRDEM | 2 | Weak |
| | NO: 282) | NO: 1259) | 112) | (SEQ ID NO: 947) | | |
| | | | | DENRDGKVSRDEL | | |
| | | | | (SEQ ID NO: 948) | | |
| t349815 | (SEQ ID | (SEQ ID | (221,233) | DHDGNGEIDLDEW | 1 | Weak |
| | NO: 283) | NO: 1260) | | (SEQ ID NO: 949) | | |
| t349833 | (SEQ ID | (SEQ ID | (520, 532)(547, | DADGDGRVEARKR | 2 | Weak |
| | NO: 284) | NO: 1261) | 559) | (SEQ ID NO: 950) | | |
| | | | | DTNGDGLFDYRET | | |
| | | | | (SEQ ID NO: 951) | | |
| t350043 | (SEQ ID | (SEQ ID | (137, 149) | CPERDGRIDLDEF | 1 | Weak |
| | NO: 285) | NO: 1262) | | (SEQ ID NO: 952) | | |
| t349829 | (SEQ ID | (SEQ ID | (221,233) | DLDGNGELDLQEW | 1 | Weak |
| | NO: 286) | NO: 1263) | | (SEQ ID NO: 953) | | |
| t349769 | (SEQ ID | (SEQ ID | (70, 82)(94, 106) | DRNRDRVITRDEM | 3 | Weak |
| | NO: 287) | NO: 1264) | (118, 130) | (SEQ ID NO: 954) | | |
| | | | | DVDGNNLLTFEEW | | |
| | | | | (SEQ ID NO: 955) | | |
| | | | | DGNGDGRLTPREF | | |
| | | | | (SEQ ID NO: 956) | | |
| t349855 | (SEQ ID | (SEQ ID | (141, 153)(83, | DADADGRVTRGEL | 2 | Weak |
| | NO: 288) | NO: 1265) | 95) | (SEQ ID NO: 957) | | |
| | | | | DRDGDGRLSVDEL | | |
| | | | | (SEQ ID NO: 958) | | |
| t349903 | (SEQ ID | (SEQ ID | (75,87)(99, 111) | DADGDGMLSRAEI | 4 | Weak |
| | NO: 289) | NO: 1266) | (131, 143)(155, | (SEQ ID NO: 959) | | |
| | | | 167) | DTDGDGAVTEAEM | | |
| | | | | (SEQ ID NO: 960) | | |
| | | | | DANGDGRIDAGEM | | |
| | | | | (SEQ ID NO: 961) | | |
| | | | | DADGDGMVSRDEM | | |
| | | | | (SEQ ID NO: 962) | | |
| t349880 | (SEQ ID | (SEQ ID | (217, 229)(151, | DEDRDGFLTRADI | 6 | Weak |
| | NO: 290) | NO: 1267) | 163)(126, 138) | (SEQ ID NO: 963) | | |
| | | | (84, 96)(58, 70) | DLNRDGYLVASEV | | |
| | | | (190, 202) | (SEQ ID NO: 964) | | |
| | | | | DTDGDGRISKLEA | | |
| | | | | (SEQ ID NO: 965) | | |
| | | | | DKNGDGQLDAGER | | |
| | | | | (SEQ ID NO: 966) | | |
| | | | | ANQDGVIDRSEA | | |
| | | | | (SEQ ID NO: 967) | | |
| | | | | DTNADGRLDRAEV | | |
| | | | | (SEQ ID NO: 968) | | |
| t349901 | (SEQ ID | (SEQ ID | (106, 118)(45, | DGNGDGYLSRAEF | 3 | Weak |
| | NO: 291) | NO: 1268) | 57)(69,81) | (SEQ ID NO: 969) | | |
| | | | | DLDGDGRISLAEF | | |
| | | | | (SEQ ID NO: 970) | | |
| | | | | DLDGNDVLEPHEQ | | |
| | | | | (SEQ ID NO: 971) | | |
| t349895 | (SEQ ID | (SEQ ID | (58, 70) | DADGNGYIDYVGS | 1 | Weak |
| | NO: 292) | NO: 1269) | | (SEQ ID NO: 972) | | |
| t349789 | (SEQ ID | (SEQ ID | (58, 70) | DVDGNGYIDYVGS | 1 | Weak |
| | NO: 293) | NO: 1270) | | (SEQ ID NO: 973) | | |
| t349972 | (SEQ ID | (SEQ ID | (423, 435) | DLDGDGEIDMREW | 1 | Weak |
| | NO: 294) | NO: 1271) | | (SEQ ID NO: 974) | | |
| t349973 | (SEQ ID | (SEQ ID | (459, 471)(486, | DLDHDGVLTIFDY | 2 | Weak |
| | NO: 295) | NO: 1272) | 498) | (SEQ ID NO: 975) | | |
| | | | | DFDGDGELTLFDF | | |
| | | | | (SEQ ID NO: 976) | | |
| t349807 | (SEQ ID | (SEQ ID | (109, 121) | DKDQDGTISVRTT | 1 | Weak |
| | NO: 296) | NO: 1273) | | (SEQ ID NO: 977) | | |
| t339616 | (SEQ ID | (SEQ ID | | | 0 | Weak |
| | NO: 297) | NO: 1274) | | | | |
| TRFE_ | (SEQ ID | | | | 0 | Iron |
| HUMAN2 | NO: 298) | | | | | metal |
| | | | | | | binding |
| | | | | | | control |
| | | | | | | protein |
| TRFE_ | (SEQ ID | | | | 0 | Iron |
| BOVIN | NO: 299) | | | | | metal |
| 2 | | | | | | binding |
| | | | | | | control |
| | | | | | | protein |

TABLE 3
Results of metal-binding assays.

| | SEQ | | | | | | |
|---|---|---|---|---|---|---|---|
| | ID | | Tb_Intensity/ | | Dy_Intensity/ | | Fe_Intensity/ |
| Strain | NO: | Tb_Intensity | [Protein] | Dy_Intensity | [Protein] | Fe_Intensity | [Protein] |
| t406938 | 1 | 3293 | 28.9 | 1043 | 6.4 | | |
| t347901 | 2 | 1745 | 11.3 | 697 | 3.1 | 0.044 | 2.01E−05 |
| t347902 | 3 | 1593 | 11.3 | 601 | 2.7 | 0.044 | 3.29E−05 |
| t349871 | 4 | 1672 | 43.7 | 587 | 6.0 | 0.044 | 8.23E−05 |
| t349865 | 5 | 4045 | 43.8 | 1097 | 8.4 | 0.044 | 2.96E−05 |
| t349953 | 6 | 2016 | 39.0 | 855 | 12.7 | 0.044 | 7.66E−05 |
| t349867 | 7 | 2404 | 81.3 | 824 | 18.8 | 0.044 | 1.19E−04 |
| t350021 | 8 | 2544 | 16.3 | 773 | 3.8 | | |
| t349910 | 9 | 2355 | 16.5 | 766 | 3.2 | 0.043 | 1.36E−05 |
| t349766 | 10 | 2470 | 71.8 | 760 | 10.3 | 0.043 | 3.66E−05 |
| t349817 | 11 | 2238 | 118.6 | 750 | 33.1 | 0.046 | 4.07E−04 |
| t349905 | 12 | 1654 | 98.2 | 738 | 21.6 | 0.045 | 2.12E−04 |
| t350032 | 13 | 2167 | 138.4 | 736 | 22.2 | 0.045 | 4.12E−04 |
| t349904 | 14 | 1923 | 153.7 | 714 | 24.9 | 0.043 | 2.38E−04 |
| t349917 | 15 | 1599 | 130.8 | 682 | 15.1 | 0.044 | 1.92E−04 |
| t349989 | 16 | 2166 | 36.1 | 681 | 7.5 | 0.044 | 5.99E−05 |
| t349986 | 17 | 2241 | 21.3 | 668 | 4.9 | 0.045 | 6.89E−05 |
| t350040 | 18 | 1735 | 127.8 | 659 | 41.5 | 0.044 | 4.58E−04 |
| t349942 | 19 | 2053 | 38.8 | 650 | 16.2 | 0.045 | 1.49E−04 |
| t349798 | 20 | 1647 | 52.7 | 648 | 14.4 | 0.045 | 2.27E−04 |
| t349843 | 21 | 2371 | 107.5 | 646 | 18.3 | 0.043 | 1.22E−04 |
| t349820 | 22 | 1812 | 37.0 | 607 | 7.8 | 0.044 | 8.71E−05 |
| t349761 | 23 | 1687 | 94.9 | 605 | 32.4 | 0.043 | 2.94E−04 |
| t349957 | 24 | 1566 | 97.0 | 574 | 20.3 | 0.044 | 2.81E−04 |
| t349887 | 25 | 1497 | 98.3 | 567 | 27.0 | 0.043 | 1.52E−04 |
| t350029 | 26 | 1666 | 51.9 | 542 | 15.3 | 0.042 | 7.53E−06 |
| t347905 | 27 | 2444 | 15.5 | | | | |
| t349915 | 28 | 2263 | 258.0 | | | | |
| t349893 | 29 | 2221 | 583.9 | | | | |
| t349889 | 30 | 2197 | 773.8 | | | | |
| t349961 | 31 | 2134 | 362.5 | | | | |
| t349916 | 32 | 1983 | 240.3 | | | | |
| t349765 | 33 | 1970 | 91.4 | | | 0.045 | 2.60E−04 |
| t349909 | 34 | 1960 | 414.9 | | | | |
| t349980 | 35 | 1948 | 227.4 | | | | |
| t349928 | 36 | 1929 | 241.2 | | | | |
| t349764 | 37 | 1904 | 31.1 | | | | |
| t349937 | 38 | 1898 | 90.3 | | | | |
| t349936 | 39 | 1896 | 12.4 | | | | |
| t349954 | 40 | 1881 | 15.8 | | | | |
| t349797 | 41 | 1868 | 357.6 | | | | |
| t349931 | 42 | 1852 | 379.9 | | | | |
| t349866 | 43 | 1846 | 279.0 | | | | |
| t350037 | 44 | 1807 | 304.1 | | | | |
| t349819 | 45 | 1792 | 180.6 | | | | |
| t350030 | 46 | 1790 | 13.7 | | | | |
| t349933 | 47 | 1770 | 348.6 | | | | |
| t349939 | 48 | 1751 | 379.3 | | | | |
| t350035 | 49 | 1728 | 297.9 | | | | |
| t349818 | 50 | 1726 | 6.3 | | | | |
| t349835 | 51 | 1719 | 160.5 | | | | |
| t349981 | 52 | 1711 | 13.1 | | | | |
| t349846 | 53 | 1701 | 154.2 | | | | |
| t349944 | 54 | 1697 | 672.5 | | | | |
| t349874 | 55 | 1696 | 74.7 | | | | |
| t350022 | 56 | 1693 | 81.0 | | | | |
| t349979 | 57 | 1693 | 9.4 | | | | |
| t350009 | 58 | 1674 | 446.0 | | | | |
| t349785 | 59 | 1661 | 151.3 | | | | |
| t350004 | 60 | 1660 | 71.8 | | | | |
| t349914 | 61 | 1651 | 550.1 | | | | |
| t349840 | 62 | 1650 | 84.0 | | | | |
| t350033 | 63 | 1645 | 859.8 | | | | |
| t349804 | 64 | 1639 | 196.6 | | | | |
| t349913 | 65 | 1633 | 471.1 | | | | |
| t349985 | 66 | 1625 | 368.5 | | | | |
| t349872 | 67 | 1622 | 105.3 | | | | |
| t349778 | 68 | 1616 | 291.9 | | | | |
| t349823 | 69 | 1614 | 329.3 | | | | |
| t349844 | 70 | 1607 | 555.0 | | | | |
| t349805 | 71 | 1606 | 6.0 | | | | |
| t349853 | 72 | 1588 | 334.6 | | | | |
| t349828 | 73 | 1584 | 8.0 | | | | |
| t350014 | 74 | 1583 | 11.5 | | | | |
| t350031 | 75 | 1578 | 467.6 | | | | |
| t349998 | 76 | 1578 | 10.9 | | | | |
| t349808 | 77 | 1572 | 224.3 | | | | |
| t349968 | 78 | 1556 | 219.0 | | | | |
| t349918 | 79 | 1556 | 62.5 | | | | |
| t349786 | 80 | 1555 | 5.2 | | | | |
| t350007 | 81 | 1554 | 92.4 | | | | |
| t349949 | 82 | 1546 | 28.0 | | | | |
| t349827 | 83 | 1539 | 18.9 | | | | |
| t349930 | 84 | 1537 | 41.5 | | | | |
| t349790 | 85 | 1521 | 10.4 | | | | |
| t349825 | 86 | 1517 | 439.7 | | | | |
| t349795 | 87 | 1498 | 124.5 | | | | |
| 350011 | 88 | 1496 | 387.6 | | | | |
| t349775 | 89 | 1493 | 66.1 | | | | |
| t349935 | 90 | 1490 | 128.8 | | | | |
| t350005 | 91 | 1490 | 44.5 | | | | |
| t349934 | 92 | 1490 | 9.1 | | | | |
| t349964 | 93 | 1484 | 25.6 | | | | |
| t349958 | 94 | 1481 | 158.1 | | | | |
| t349888 | 95 | 1476 | 105.8 | | | | |
| t349849 | 96 | 1473 | 69.1 | | | | |
| t349988 | 97 | 1473 | 5.7 | | | | |
| t349992 | 98 | 1472 | 248.9 | | | | |
| t349822 | 99 | 1472 | 138.2 | | | | |
| t349760 | 100 | 1471 | 401.6 | | | | |
| t349991 | 101 | 1458 | 172.1 | | | | |
| t349890 | 102 | 1456 | 165.4 | | | | |
| t349959 | 103 | 1453 | 22.5 | | | | |
| t349877 | 104 | 1443 | 89.1 | | | | |
| t349892 | 105 | 1440 | 64.9 | | | | |
| t349884 | 106 | 1425 | 642.2 | | | | |
| t349955 | 107 | 1422 | 159.6 | | | | |
| t350012 | 108 | 1415 | 228.1 | | | | |
| t349770 | 109 | 1415 | 13.6 | | | | |
| t349847 | 110 | 1413 | 215.0 | | | | |
| t349793 | 111 | 1412 | 82.8 | | | | |
| t349788 | 112 | 1412 | 61.6 | | | | |
| t349956 | 113 | 1409 | 445.3 | | | | |
| t350041 | 114 | 1405 | 354.6 | | | | |
| t350000 | 115 | 1400 | 6.5 | | | | |
| t349906 | 116 | 1397 | 45.0 | | | | |
| t349799 | 117 | 1397 | 177.0 | | | | |
| t349878 | 118 | 1393 | 6.8 | | | | |
| t349922 | 119 | 1384 | 140.9 | | | | |
| t350003 | 120 | 1383 | 156.4 | | | | |
| t349990 | 121 | 1383 | 97.4 | | | | |
| t349950 | 122 | 1382 | 23.8 | | | | |
| t349791 | 123 | 1381 | 546.3 | | | | |
| t347903 | 124 | 1380 | 11.4 | | | | |
| t349845 | 125 | 1380 | 201.2 | | | | |
| t349812 | 126 | 1368 | 130.8 | | | | |
| t350036 | 127 | 1367 | 28.0 | | | | |
| t349908 | 128 | 1367 | 54.9 | | | | |
| t349783 | 129 | 1365 | 40.2 | | | | |
| t349873 | 130 | 1355 | 214.3 | | | | |
| t350006 | 131 | 1354 | 651.6 | | | | |
| t349870 | 132 | 1352 | 27.5 | | | | |
| t349868 | 133 | 1351 | 231.1 | | | | |
| t349839 | 134 | 1348 | 80.7 | | | | |
| t349838 | 135 | 1346 | 32.3 | | | | |
| t349898 | 136 | 1339 | 110.4 | | | | |
| t350045 | 137 | 1335 | 82.3 | | | | |
| t349806 | 138 | 1334 | 77.5 | | | | |
| t349842 | 139 | 1328 | 79.5 | | | | |
| t349824 | 140 | 1325 | 32.0 | | | | |
| t349857 | 141 | 1319 | 65.4 | | | | |
| t347907 | 142 | 1316 | 103.2 | | | | |
| t349768 | 143 | 1312 | 8.9 | | | | |
| t350034 | 144 | 1311 | 46.4 | | | | |
| t349802 | 145 | 1307 | 255.2 | | | | |
| t349941 | 146 | 1306 | 196.5 | | | | |
| t349932 | 147 | 1306 | 48.1 | | | | |
| t349869 | 148 | 1303 | 115.3 | | | | |
| t349927 | 149 | 1299 | 13.2 | | | | |
| t350039 | 150 | 1296 | 199.5 | | | | |
| t349965 | 151 | 1291 | 364.7 | | | | |
| t349897 | 152 | 1290 | 391.6 | | | | |
| t349767 | 153 | 1290 | 7.6 | | | | |
| t349999 | 154 | 1289 | 6.8 | | | | |
| t349850 | 155 | 1289 | 133.6 | | | | |
| t349963 | 156 | 1282 | 45.1 | | | | |
| t349891 | 157 | 1280 | 161.6 | | | | |
| t349830 | 158 | 1275 | 17.8 | | | | |
| t349984 | 159 | 1274 | 168.1 | | | | |
| t349848 | 160 | 1270 | 181.9 | | | | |
| t350016 | 161 | 1268 | 147.0 | | | | |
| t349800 | 162 | 1266 | 84.9 | | | | |
| t349836 | 163 | 1265 | 316.5 | | | | |
| t349860 | 164 | 1263 | 40.8 | | | | |
| t349982 | 165 | 1262 | 271.0 | | | | |
| t349781 | 166 | 1262 | 58.3 | | | | |
| t349826 | 167 | 1259 | 268.3 | | | | |
| t349792 | 168 | 1256 | 209.5 | | | | |
| t349784 | 169 | 1241 | 4.0 | | | | |
| t350042 | 170 | 1239 | 28.8 | | | | |
| t349894 | 171 | 1234 | 180.8 | | | | |
| t349809 | 172 | 1233 | 33.9 | | | | |
| t350017 | 173 | 1228 | 60.4 | | | | |
| t349929 | 174 | 1225 | 173.7 | | | | |
| t349763 | 175 | 1219 | 148.3 | | | | |
| t349978 | 176 | 1218 | 56.7 | | | | |
| t349841 | 177 | 1207 | 132.6 | | | | |
| t349943 | 178 | 1204 | 7.0 | | | | |
| t349864 | 179 | 1202 | 92.2 | | | | |
| t349947 | 180 | 1199 | 453.8 | | | | |
| t350025 | 181 | 1187 | 279.0 | | | | |
| t349762 | 182 | 1186 | 53.2 | | | | |
| t349900 | 183 | 1186 | 33.4 | | | | |
| t350013 | 184 | 1184 | 78.4 | | | | |
| t350026 | 185 | 1183 | 62.3 | | | | |
| t349861 | 186 | 1180 | 41.9 | | | | |
| t349852 | 187 | 1179 | 58.9 | | | | |
| 349919 | 188 | 1175 | 386.2 | | | | |
| t349811 | 189 | 1174 | 182.4 | | | | |
| t349772 | 190 | 1173 | 235.4 | | | | |
| t350038 | 191 | 1173 | 2.8 | | | | |
| t350048 | 192 | 1166 | 204.6 | | | | |
| 349854 | 193 | 1165 | 25.6 | | | | |
| t349780 | 194 | 1164 | 402.2 | | | | |
| t349814 | 195 | 1158 | 200.1 | | | | |
| t349803 | 196 | 1154 | 449.9 | | | | |
| t349794 | 197 | 1154 | 202.4 | | | | |
| t350008 | 198 | 1151 | 9.6 | | | | |
| t349951 | 199 | 1151 | 138.3 | | | | |
| t349831 | 200 | 1150 | 91.8 | | | | |
| t349946 | 201 | 1143 | 221.2 | | | | |
| t349782 | 202 | 1142 | 33.1 | | | | |
| t350028 | 203 | 1137 | 109.5 | | | | |
| t349993 | 204 | 1130 | 189.6 | | | | |
| t349970 | 205 | 1129 | 3.3 | | | | |
| t349863 | 206 | 1126 | 223.2 | | | | |
| t349975 | 207 | 1125 | 9.4 | | | | |
| t349926 | 208 | 1124 | 22.7 | | | | |
| t349810 | 209 | 1124 | 52.1 | | | | |
| t350018 | 210 | 1122 | 121.3 | | | | |
| t349896 | 211 | 1120 | 161.9 | | | | |
| t349966 | 212 | 1120 | 30.8 | | | | |
| t349876 | 213 | 1120 | 63.6 | | | | |
| t349911 | 214 | 1118 | 11.0 | | | | |
| t350023 | 215 | 1118 | 5.0 | | | | |
| t349837 | 216 | 1106 | 252.5 | | | | |
| t349923 | 217 | 1100 | 42.0 | | | | |
| t347906 | 218 | 1094 | 2.8 | | | | |
| t349976 | 219 | 1093 | 57.5 | | | | |
| t349883 | 220 | 1084 | 5.9 | | | | |
| t349886 | 221 | 1084 | 11.7 | | | | |
| t349974 | 222 | 1084 | 200.8 | | | | |
| t349813 | 223 | 1083 | 581.7 | | | | |
| t349969 | 224 | 1083 | 69.3 | | | | |
| t349759 | 225 | 1080 | 34.7 | | | | |
| t349862 | 226 | 1077 | 4.1 | | | | |
| t349912 | 227 | 1077 | 101.8 | | | | |
| t349774 | 228 | 1075 | 69.4 | | | | |
| t349879 | 229 | 1069 | 23.7 | | | | |
| t349885 | 230 | 1067 | 23.4 | | | | |
| t349858 | 231 | 1058 | 131.8 | | | | |
| t349987 | 232 | 1057 | 40.0 | | | | |
| t349977 | 233 | 1056 | 189.8 | | | | |
| t349925 | 234 | 1055 | 11.3 | | | | |
| t349960 | 235 | 1049 | 1688.3 | | | | |
| t349995 | 236 | 1048 | 188.6 | | | | |
| t349920 | 237 | 1043 | 3.6 | | | | |
| t349997 | 238 | 1039 | 12.8 | | | | |
| t349907 | 239 | 1037 | 195.5 | | | | |
| t349771 | 240 | 1036 | 98.7 | | | | |
| t349899 | 241 | 1035 | 102.8 | | | | |
| t349816 | 242 | 1033 | 61.1 | | | | |
| t349945 | 243 | 1029 | 184.0 | | | | |
| t349902 | 244 | 1026 | 7.0 | | | | |
| t350027 | 245 | 1024 | 114.3 | | | | |
| t349994 | 246 | 1017 | 46.8 | | | | |
| t349938 | 247 | 1011 | 157.2 | | | | |
| t349859 | 248 | 1008 | 27.9 | | | | |
| t349787 | 249 | 1005 | 24.6 | | | | |
| t350001 | 250 | 1005 | 83.4 | | | | |
| t349921 | 251 | 1002 | 28.2 | | | | |
| t350019 | 252 | 997 | 4.5 | | | | |
| t350002 | 253 | 994 | 428.4 | | | | |
| t349832 | 254 | 993 | 286.6 | | | | |
| t347904 | 255 | 983 | 6.6 | | | | |
| t349971 | 256 | 983 | 106.3 | | | | |
| t349834 | 257 | 981 | 517.5 | | | | |
| t350024 | 258 | 977 | 340.5 | | | | |
| t349856 | 259 | 968 | 17.6 | | | | |
| t349801 | 260 | 959 | 33.8 | | | | |
| t349924 | 261 | 959 | 35.8 | | | | |
| t349851 | 262 | 956 | 30.3 | | | | |
| t350047 | 263 | 951 | 1880.0 | | | | |
| t350044 | 264 | 941 | 57.1 | | | | |
| t349777 | 265 | 936 | 144.8 | | | | |
| t349773 | 266 | 933 | 14.7 | | | | |
| t349796 | 267 | 928 | 19.6 | | | | |
| t350010 | 268 | 923 | 13.7 | | | | |
| t349962 | 269 | 920 | 14.2 | | | | |
| t349875 | 270 | 918 | 33.9 | | | | |
| t349881 | 271 | 914 | 27.9 | | | | |
| t349940 | 272 | 912 | 13.6 | | | | |
| t349948 | 273 | 912 | 60.4 | | | | |
| t349882 | 274 | 906 | 57.4 | | | | |
| t349996 | 275 | 903 | 3.8 | | | | |
| t349967 | 276 | 902 | 11.8 | | | | |
| t350046 | 277 | 900 | 66.9 | | | | |
| t349776 | 278 | 894 | 56.5 | | | | |
| t349779 | 279 | 892 | 56.6 | | | | |
| t350020 | 280 | 890 | 31.4 | | | | |
| t349952 | 281 | 882 | 6.1 | | | | |
| t350015 | 282 | 878 | 5.4 | | | | |
| t349815 | 283 | 876 | 386.3 | | | | |
| t349833 | 284 | 858 | 430.4 | | | | |
| t350043 | 285 | 845 | 42.4 | | | | |
| t349829 | 286 | 820 | 130.3 | | | | |
| t349769 | 287 | 808 | 26.7 | | | | |
| t349855 | 288 | 800 | 42.5 | | | | |
| t349903 | 289 | 788 | 1.7 | | | | |
| t349880 | 290 | 787 | 11.4 | | | | |
| t349901 | 291 | 782 | 8.4 | | | | |
| t349895 | 292 | 776 | 29.0 | | | | |
| t349789 | 293 | 759 | 94.1 | | | | |
| t349972 | 294 | 759 | 174.6 | | | | |
| t349973 | 295 | 741 | 438.9 | | | | |
| t349807 | 296 | 690 | 29.3 | | | | |
| t339616 | 297 | | | | | 0.043 | 1.32E−04 |
| TRFE_ | 298 | | | | | 0.053 | 1.57E−03 |
| HUMAN2 | | | | | | | |
| TRFE_ | 299 | | | | | 0.046 | 5.72E−04 |
| BOVIN2 | | | | | | | |

### EQUIVALENTS

Those skilled in the art will recognize, or be able to ascertain using no more than routine experimentation, many equivalents to the specific embodiments of the invention described in this disclosure. Such equivalents are intended to be encompassed by the following claims.

All references, including patent documents, disclosed in this application are incorporated by reference in their entirety, particularly for the disclosure referenced in this disclosure.

1. A method of recovering a rare earth element (REE) from a sample comprising: introducing an REE-binding protein to the sample, wherein the REE-binding protein comprises an EF-hand domain, and recovering the REE from the sample.

2. The method of claim 1, further comprising purifying an REE from the sample.

3. The method of claim 1 or 2, wherein the EF-hand domain comprises the sequence:
X₁X₂X₃X₄X₅X₆X₇X₈X₉X₁₀X₁₁X₁₂ (SEQ ID NO: 1275),
wherein X denotes any amino acid;
wherein X₁ is D or K;
wherein X₂ is A, D, E, F, G, H, I, K, L, P, Q, R, S, T, V, or Y;
wherein X₃ is D, E, N, Q, S, or T;
wherein X₄ is A, D, E, G, H, K, L, N, Q, R, S, or T;
wherein X₅ is D, N, S, or T;
wherein X₆ is A, E, G, K, N, Q, R, or S;
wherein X₇ is A, D, E, F, G, H, I, K, L, M, N, Q, R, S, T, V, or Y;
wherein X₈ is A, F, I, L, M, or V;
wherein X₉ is A, D, E, G, L, N, Q, S, T, or V;
wherein X₁₀ is A, D, E, F, G, H, I, K, L, M, N, P, Q, R, S, T, V, or Y;
wherein X₁₁ is A, D, E, F, G, I, K, L, M, N, Q, R, S, T, V, or Y; and
wherein X₁₂ is A, D, E, G, I, K, L, Q, R, S, T, or V.

4. The method of claim 3, wherein the EF-hand domain additionally comprises X₁₃,
wherein X₁₃ is A, E, F, G, H, I, L, M, Q, R, S, T, V, W, or Y.

5. The method of claim 1 or 2, wherein the EF-hand domain comprises the sequence:
DX₁X₂X₃X₄X₅X₆X₇X₈X₉X₁₀X₁₁ (SEQ ID NO: 1276),
wherein X₁ is A, D, F, G, I, K, L, P, Q, R, S, T, V, or Y;
wherein X₂ is D, N, Q, or S;
wherein X₃ is A, D, G, H, K, N, Q, R, or S;
wherein X₄ is D, N, or S;
wherein X₅ is A, E, G, K, N, Q, or R;
wherein X₆ is A, D, E, F, H, I, K, L, M, N, Q, R, S, T, V, or Y;
wherein X₇ is A, F, I, L, or V;
wherein X₈ is D, E, G, N, S, T, or V;
wherein X₉ is A, D, E, F, G, H, I, K, L, M, N, P, Q, R, S, T, V, or Y;
wherein X₁₀ is A, D, E, F, G, I, K, L, N, Q, R, S, T, V, or Y; and
wherein X₁₁ is A, D, E, G, L, Q, R, S, T, or V.

6. The method of claim 5, wherein the EF-hand domain additionally comprises X₁₂,
wherein X₁₂ is A, E, F, G, H, I, L, M, Q, R, S, V, W, or Y.

7. The method of claim 1 or 2, wherein the EF-hand domain comprises the sequence:
DX₁X₂X₃X₄X₅X₆X₇X₈X₉X₁₀X₁₁ (SEQ ID NO: 1277),
wherein X₁ is A, D, F, G, I, K, L, Q, R, S, T, V, or Y;
wherein X₂ is D, N, Q, or S;
wherein X₃ is A, D, G, H, K, N, Q, R, or S;
wherein X₄ is D, N, or S;
wherein X₅ is A, E, G, K, N, Q, or R;
wherein X₆ is A, D, E, F, H, I, K, L, M, N, Q, R, S, T, V, or Y;
wherein X₇ is A, F, I, L, or V;
wherein X₈ is D, E, G, N, S, T, or V;
wherein X₉ is A, D, E, F, G, H, I, K, L, M, N, P, Q, R, S, T, V, or Y;
wherein X₁₀ is A, D, E, F, G, I, K, L, N, Q, R, S, T, V, or Y; and
wherein X₁₁ is A, D, E, G, L, Q, R, S, T, or V.

8. The method of claim 7, wherein the EF-hand domain additionally comprises X₁₂,
wherein X₁₂ is A, E, F, G, H, I, L, M, Q, R, S, V, W, or Y.

9. The method of any one of claims 1-8, wherein the EF-hand domain comprises no more than two amino acid substitutions relative to an EF-hand domain sequence in Table 2, corresponding to SEQ ID NOS: 300-977.

10. The method of claim 9, wherein the EF-hand domain comprises a sequence selected from the EF-hand domain sequences in Table 2, corresponding to SEQ ID NOS: 300-977.

11. The method of claim 10, wherein the EF-hand domain comprises a sequence selected from SEQ ID NOs: 304-715.

12. The method of claim 11, wherein the EF-hand domain comprises a sequence selected from SEQ ID NOs: 304-473.

13. The method of any one of claims 1-12, wherein the REE binding protein comprises at least two EF-hand domain sequences.

14. The method of claim 13, wherein the REE binding protein comprises at least three or at least four EF-hand domain sequences.

15. The method of claim 13, wherein the at least two EF-hand domain sequences are different.

16. The method of any one of claims 1-15, wherein the REE binding protein is a lanthanide binding protein, a yttrium binding protein, and/or a scandium binding protein.

17. The method of claim 16, wherein the REE binding protein has at least 10%, 20%, 30%, 40%, 50%, 60%, 70%, 80%, 90%, or 100% higher lanthanide binding affinity relative to a control protein, wherein the control protein comprises SEQ ID NO: 2 or SEQ ID NO: 3.

18. The method of claim 17, wherein the binding affinity is determined using a dialysis assay.

19. The method of any one of claims 16-18, wherein the lanthanide is selected from the group consisting of cerium (Ce), dysprosium (Dy), erbium (Er), europium (Eu), gadolinium (Gd), holmium (Ho), lanthanum (La), lutetium (Lu), neodymium (Nd), praseodymium (Pr), promethium (Pm), samarium (Sm), terbium (Tb), thulium (Tm), ytterbium (Yb), and a combination thereof.

20. The method of any one of claims 1-19, wherein the REE binding protein has at least 10%, 20%, 30%, 40%, 50%, 60%, 70%, 80%, 90%, or 100% lower iron binding affinity relative to a control protein, wherein the control protein is TRFE_HUMAN2 (SEQ ID NO: 298) or TRFE_BOVIN2 (SEQ ID NO: 299).

21. The method of claim 20, wherein the binding affinity is determined using a dialysis assay.

22. The method of any one of claims 1-21, wherein the REE binding protein is expressed as a heterologous gene in a host cell.

23. The method of claim 22, wherein the REE binding protein is secreted from the host cell.

24. The method of any one of claims 1-23, wherein the REE-binding protein comprises a sequence that is at least 90% identical to any one of SEQ ID NOs: 4-71.

25. The method of claim 24, wherein the REE-binding protein comprises any one of SEQ ID NOs: 4-71.

26. The method of any one of claims 1-25, wherein the REE-binding protein does not comprise SEQ ID NO: 1.

27. The method of any one of claims 1-26, wherein the REE-binding protein does not comprise SEQ ID NO: 2 or SEQ ID NO: 3.

28. The method of any one of claims 1-27, wherein the REE-binding protein comprises at least 90% identity to the portion of a sequence selected from any one of SEQ ID NOs: 4-71 outside of the EF hand domains within the sequence, and wherein the REE binding protein comprises no more than two amino acid substitutions relative to the EF-hand domain(s) within the sequence.

29. A host cell comprising a heterologous gene expressing a rare earth element (REE) binding protein, wherein the REE binding protein comprises an EF-hand domain, wherein the REE binding protein does not comprise SEQ ID NO: 1.

30. The host cell of claim 29, wherein the EF-hand domain comprises the sequence:
X₁X₂X₃X₄X₅X₆X₇X₈X₉X₁₀X₁₁X₁₂ (SEQ ID NO: 1275),
wherein X denotes any amino acid;
wherein X₁ is D or K;
wherein X₂ is A, D, E, F, G, H, I, K, L, P, Q, R, S, T, V, or Y;
wherein X₃ is D, E, N, Q, S, or T;
wherein X₄ is A, D, E, G, H, K, L, N, Q, R, S, or T;
wherein X₅ is D, N, S, or T;
wherein X₆ is A, E, G, K, N, Q, R, or S;
wherein X₇ is A, D, E, F, G, H, I, K, L, M, N, Q, R, S, T, V, or Y;
wherein X₈ is A, F, I, L, M, or V;
wherein X₉ is A, D, E, G, L, N, Q, S, T, or V;
wherein X₁₀ is A, D, E, F, G, H, I, K, L, M, N, P, Q, R, S, T, V, or Y;
wherein X₁₁ is A, D, E, F, G, I, K, L, M, N, Q, R, S, T, V, or Y; and
wherein X₁₂ is A, D, E, G, I, K, L, Q, R, S, T, or V.

31. The host cell of claim 30, wherein the EF-hand domain additionally comprises X₁₃,
wherein X₁₃ is A, E, F, G, H, I, L, M, Q, R, S, T, V, W, or Y.

32. The host cell of claim 29, wherein the EF-hand domain comprises the sequence:
DX₁X₂X₃X₄X₅X₆X₇X₈X₉X₁₀X₁₁ (SEQ ID NO: 1276),
wherein X₁ is A, D, F, G, I, K, L, P, Q, R, S, T, V, or Y;
wherein X₂ is D, N, Q, or S;
wherein X₃ is A, D, G, H, K, N, Q, R, or S;
wherein X₄ is D, N, or S;
wherein X₅ is A, E, G, K, N, Q, or R;
wherein X₆ is A, D, E, F, H, I, K, L, M, N, Q, R, S, T, V, or Y;
wherein X₇ is A, F, I, L, or V;
wherein X₈ is D, E, G, N, S, T, or V;
wherein X₉ is A, D, E, F, G, H, I, K, L, M, N, P, Q, R, S, T, V, or Y;
wherein X₁₀ is A, D, E, F, G, I, K, L, N, Q, R, S, T, V, or Y; and
wherein X₁₁ is A, D, E, G, L, Q, R, S, T, or V.

33. The host cell of claim 32, wherein the EF-hand domain additionally comprises X₁₂,
wherein X₁₂ is A, E, F, G, H, I, L, M, Q, R, S, V, W, or Y.

34. The host cell of claim 29, wherein the EF-hand domain comprises the sequence:
DX₁X₂X₃X₄X₅X₆X₇X₈X₉X₁₀X₁₁ (SEQ ID NO: 1277),
wherein X₁ is A, D, F, G, I, K, L, Q, R, S, T, V, or Y;
wherein X₂ is D, N, Q, or S;
wherein X₃ is A, D, G, H, K, N, Q, R, or S;
wherein X₄ is D, N, or S;
wherein X₅ is A, E, G, K, N, Q, or R;
wherein X₆ is A, D, E, F, H, I, K, L, M, N, Q, R, S, T, V, or Y;
wherein X₇ is A, F, I, L, or V;
wherein X₈ is D, E, G, N, S, T, or V;
wherein X₉ is A, D, E, F, G, H, I, K, L, M, N, P, Q, R, S, T, V, or Y;
wherein X₁₀ is A, D, E, F, G, I, K, L, N, Q, R, S, T, V, or Y; and
wherein X₁₁ is A, D, E, G, L, Q, R, S, T, or V.

35. The host cell of claim 34, wherein the EF-hand domain additionally comprises X₁₂,
wherein X₁₂ is A, E, F, G, H, I, L, M, Q, R, S, V, W, or Y.

36. The host cell of any one of claims 26-35, wherein the EF-hand domain comprises no more than two amino acid substitutions relative to a sequence selected from the EF-hand domain sequences in Table 2, corresponding to SEQ ID NOs: 300-977.

37. The host cell of claim 36, wherein the EF-hand domain comprises a sequence selected from the EF-hand domain sequences in Table 2, corresponding to SEQ ID NOs: 300-977.

38. The host cell of claim 37, wherein the EF-hand domain comprises a sequence selected from SEQ ID NOs: 304-715.

39. The host cell of claim 38, wherein the EF-hand domain comprises a sequence selected from SEQ ID NOs: 304-473.

40. The host cell of any one of claims 29-39, wherein the REE binding protein comprises at least two EF-hand domain sequences.

41. The host cell of claim 40, wherein the REE binding protein comprises at least three or at least four EF-hand domain sequences.

42. The host cell of claim 40, wherein the at least two EF-hand domain sequences are different.

43. The host cell of any one of claims 29-42, wherein the REE binding protein is a lanthanide binding protein, a yttrium binding protein, and/or a scandium binding protein.

44. The host cell of claim 43, wherein the REE binding protein has at least 10%, 20%, 30%, 40%, 50%, 60%, 70%, 80%, 90%, or 100% higher lanthanide binding affinity relative to a control protein, wherein the control protein comprises SEQ ID NO: 2 or SEQ ID NO: 3.

45. The host cell of claim 44, wherein the binding affinity is determined using a dialysis assay.

46. The host cell of any one of claims 43-45, wherein the lanthanide is selected from the group consisting of cerium (Ce), dysprosium (Dy), erbium (Er), europium (Eu), gadolinium (Gd), holmium (Ho), lanthanum (La), lutetium (Lu), neodymium (Nd), praseodymium (Pr), promethium (Pm), samarium (Sm), terbium (Tb), thulium (Tm), ytterbium (Yb), and a combination thereof.

47. The host cell of any one of claims 29-46, wherein the REE binding protein has at least 10%, 20%, 30%, 40%, 50%, 60%, 70%, 80%, 90%, or 100% lower iron binding affinity relative to a control protein, wherein the control protein is TRFE_HUMAN2 (SEQ ID NO: 298) or TRFE_BOVIN2 (SEQ ID NO: 299).

48. The host cell of claim 47, wherein the iron binding affinity is determined using a dialysis assay.

49. The host cell of any one of claims 29-48, wherein the REE-binding protein comprises a sequence that is at least 90% identical to any one of SEQ ID NOs: 4-71.

50. The host cell of claim 49, wherein the REE-binding protein comprises any one of SEQ ID NOs: 4-71.

51. The host cell of any one of claims 29-50, wherein the REE-binding protein does not comprise SEQ ID NO: 2 or SEQ ID NO: 3.

52. The host cell of any one of claims 29-51, wherein the REE-binding protein comprises at least 90% identity to the portion of a sequence selected from any one of SEQ ID NOs: 4-71 outside of the EF hand domains within the sequence, and wherein the REE binding protein comprises no more than two amino acid substitutions relative to the EF-hand domain(s) within the sequence.

53. A kit comprising a host cell of any one of claims 29-50 and instructions for extraction of a rare earth element.

54. A kit comprising a rare earth element (REE) binding protein, wherein the REE binding protein comprises an EF-hand domain, and instructions for extraction of a rare earth element.

55. The kit of claim 53 or 54, wherein the rare earth element is lanthanide, scandium, and/or yttrium.

56. The kit of any one of claims 53-55, wherein the REE binding protein comprises a sequence that is at least 90% identical to any one of SEQ ID NOs: 4-71.

57. The kit of claim 56, wherein the REE binding protein comprises any one of SEQ ID NOs: 4-71.
